SEQUENCE LISTING (161 sequences)

SEQ ID NO: 1 - protein, 715 aa, Artificial sequence
  Synthetic peptide.
QVTLKESGPGILQPSQTLSLTCSFSGFSLSTSGMGLSWIRQPSGKGLEWLAHIYWDDDKRYNPSLKSRLTISKDTSSNQVFLKITIVDTADAATYYCARSSHYYGYGYGGYFDVWGAGTTVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASQTPTNTISVTPTNNSTPTNNSNPKPNPASIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQASQPELAEAAAKTTENPLQKIDAALAQVDALRSDLGAVQNRFNSAITNLGNTVNNLSEARSRIEDSDYATEVSNMSRAQILQAS

SEQ ID NO: 2 - protein, 797 aa, Artificial sequence [sequence omitted]

SEQ ID NO: 3 - protein, 430 aa, Artificial sequence
  Synthetic peptide.
MDLDAVRIKVDTVNAKPGDTVNIPVRFSGIPSKGIANADFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASLLTEVETYVLSIIPSGPLKAEIAQRLEDVFAGKNTDLEVLMEWLKTRPILSPLTKGILGFVFTLTVPSERGLQRRRFVQNALNGNGDPNNMDKAVKLYRKLKREITFHGAKEIALSYSAGALASCMGLIYNRMGAVTTEVAFGLVCATCEQIADSQHRSHRQMVTTTNPLIRHENRMVLASTTAKAMEQMAGSSEQAAEAMDIASQARQMVQAMRTIGTHPSSSAGLKDDLLENLQAYQKRMGVQMQRFKLEHHHHHH

SEQ ID NO: 4 - protein, 513 aa, Artificial sequence
  Synthetic peptide.
QVTLKESGPGILQPSQTLSLTCSFSGFSLSTSGMGLSWIRQPSGKGLEWLAHIYWDDDKRYNPSLKSRLTISKDTSSNQVFLKITIVDTADAATYYCARSSHYYGYGYGGYFDVWGAGTTVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASQTPTNTISVTPTNNSTPTNNSNPKPNPASCQTPTNTISVTPTNNSTPTNNSNPKPCPAS

SEQ ID NO: 5 - protein, 415 aa, Artificial sequence
  Synthetic peptide.
LDITSHHHHHHDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGVNVGDTTEPATPTTPVTTPTTTDDLDAASIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQASQPELAEAAAKTTENPLQKIDAALAQVDALRSDLGAVQNRFNSAITNLGNTVNNLSEARSRIEDSDYATEVSNMSRAQILQAS

SEQ ID NO: 6 - DNA, 1409 bp, Artificial sequence
  Synthetic oligonucleotide.
atgggcaggcttacttcttcattcttgctactgattgtccctgcatatgtcctgtcccaggttactctgaaagagtctggccctgggatattgcagccctcccagaccctcagtctgacctgttctttctctgggttttcactgagcacttctggtatgagtgtaggctggattcgtcagccttcagggaagggtctggagtggctggctcacatttggtggaatgatgataagtactataatccagtcctgaaaagccggctcacaatctccaaggagacctccaacaaccaggtattcctcaagatcgccagtgtggtctctgcagatactgccacatactactgtgctcgattctatggtaactgtcttgactactggggccaaggcaccactctcacagtctcctcggccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctgattaattaa

SEQ ID NO: 7 - protein, 466 aa, Artificial sequence
  Synthetic peptide.
MGRLTSSFLLLIVPAYVLSQVTLKESGPGILQPSQTLSLTCSFSGFSLSTSGMSVGWIRQPSGKGLEWLAHIWWNDDKYYNPVLKSRLTISKETSNNQVFLKIASVVSADTATYYCARFYGNCLDYWGQGTTLTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 8 - DNA, 705 bp, Artificial sequence
  Synthetic oligonucleotide.
atgatgtcctctgctcagttccttggtctcctgttgctctgttttcaaggtaccagatgtgatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaatattacaattaggagtcccatcaagattcagtggcagtgggtctgaaacagattattctctcaccattagcaacctggagcaagaagatattgccacttacttttgccaacagggtgattcgcttccattcacgttcggctcggggacaaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 9 - protein, 234 aa, Artificial sequence
  Synthetic peptide.
MMSSAQFLGLLLLCFQGTRCDIQMTQTTSSLSASLGDRVTISCRASQDISNYLNWYQQKPDGTVKLLIYYTSILQLGVPSRFSGSGSETDYSLTISNLEQEDIATYFCQQGDSLPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - DNA, 1403 bp, Artificial sequence
  Synthetic oligonucleotide.
atgagagcgctgattcttttgtgcctgttcacagcctttcctggtatcctgtctgatgtgcagcttcaggagtcaggacctgacctggtgaaaccttctcagtcactttcactcacctgcactgtcactggctactccatcaccagtggttatagctggcactggatccggcagtttccaggaaacaaactggaatggatgggctacatactcttcagtggtagcactaactacaacccatctctgaaaagtcgaatctctatcactcgagacacatccaagaaccagttcttcctgcagttgaattctgtgactactgaggacacagccacatatttctgtgcaagatctaactatggttcctttgcttcctggggccaagggactctggtcactgtctctgcagccaaaacaacgggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctgattaattaa

SEQ ID NO: 11 - protein, 464 aa, Artificial sequence
  Synthetic peptide.
MRALILLCLFTAFPGILSDVQLQESGPDLVKPSQSLSLTCTVTGYSITSGYSWHWIRQFPGNKLEWMGYILFSGSTNYNPSLKSRISITRDTSKNQFFLQLNSVTTEDTATYFCARSNYGSFASWGQGTLVTVSAAKTTGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 12 - DNA, 708 bp, Artificial sequence
  Synthetic oligonucleotide.
atggattttcaagtgcagattttcagcttcctgctaatcagtgcctcagtcataatatccagaggacaaattgttctcacccagtctccagcaatcatgtctgcatctccaggggagaaggtcaccatgacctgcagtgccagctcaagtgtaagtcacatgcactggtaccagcagaagtcaggcacttcccccaaaagatggatttatgacacatccagactggcttctggagtccctgctcgcttcagtggcagtgggtctgggacctcttactctctcacaatcagcagcatggaggctgaagatgctgccacttattactgccagcagtggagtagtcacccatggtcgttcggtggaggcaccaaactcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 13 - protein, 235 aa, Artificial sequence
  Synthetic peptide.
MDFQVQIFSFLLISASVIISRGQIVLTQSPAIMSASPGEKVTMTCSASSSVSHMHWYQQKSGTSPKRWIYDTSRLASGVPARFSGSGSGTSYSLTISSMEAEDAATYYCQQWSSHPWSFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - DNA, 1415 bp, Artificial sequence
  Synthetic oligonucleotide.
atggcttgggtgtggaccttgctattcctgatggcagctgcccaaagtgcccaagcacagatccagttggtgcagtctggacctgagctgaagaagcctggagagacagtcaagatctcctgcaaggcttctgggtataccttcacaaactatggaatgaactgggtgaagcaggttccaggaaaaggtttaaggtggatgggctggatggacaccttcactggagagccaacatatgctgatgacttcaagggacggtttgccttctctttggaaacctctgccagcactgcctatttgcagatcaacagcctcaaaaatgaggacacggctacttatttctgtgcaagaggggggattttacgactcaactactttgactactggggccaaggcaccactctcacagtctcctcagccaaaacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctgattaattaa

SEQ ID NO: 15 - protein, 468 aa, Artificial sequence
  Synthetic peptide.
MAWVWTLLFLMAAAQSAQAQIQLVQSGPELKKPGETVKISCKASGYTFTNYGMNWVKQVPGKGLRWMGWMDTFTGEPTYADDFKGRFAFSLETSASTAYLQINSLKNEDTATYFCARGGILRLNYFDYWGQGTTLTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 16 - DNA, 705 bp, Artificial sequence
  Synthetic oligonucleotide.
atgaagtttccttctcaacttctgctcttactgctgtttggaatcccaggcatgatatgtgacatccagatgacacaatcttcatcctccttttctgtatctctaggagacagagtcaccattacttgcaaggcaagtgaggacatatataatcggttaggctggtatcagcagaaaccaggaaatgctcctaggctcttaatatctggtgcaaccagtttggaaactggggttccttcaagattcagtggcagtggatctggaaaggattacgctctcagcattaccagtcttcagactgaagatcttgctacttattactgtcaacagtgttggacttctccgtacacgttcggaggggggaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 17 - protein, 234 aa, Artificial sequence
  Synthetic peptide.
MKFPSQLLLLLLFGIPGMICDIQMTQSSSSFSVSLGDRVTITCKASEDIYNRLGWYQQKPGNAPRLLISGATSLETGVPSRFSGSGSGKDYALSITSLQTEDLATYYCQQCWTSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - DNA, 1398 bp, Artificial sequence
  Synthetic oligonucleotide.
atgggatggagctggatctttctctttctcttgtcaggaactggaggtgtcctctctgaggtccagctgcaacagtctggacctgagctggtgaagcctggggcttcagtgaagatgtcctgcaaggcttctggatacaccttcactgactactacatgaagtgggtgaagcagagccatggaaagagccttgagtggattggagatattaatcctaactatggtgatactttctacaaccagaagttcgagggcaaggccacattgactgtagacaaatcctccaggacagcctacatgcagctcaacagcctgacatctgaggactctgcagtctattattgtggaagaggggactatggatacttcgatgtctggggcgcagggaccacggtcaccgtctcctcagccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 19 - protein, 464 aa, Artificial sequence
  Synthetic peptide.
MGWSWIFLFLLSGTGGVLSEVQLQQSGPELVKPGASVKMSCKASGYTFTDYYMKWVKQSHGKSLEWIGDINPNYGDTFYNQKFEGKATLTVDKSSRTAYMQLNSLTSEDSAVYYCGRGDYGYFDVWGAGTTVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLLSLGKAS

SEQ ID NO: 20 - DNA, 705 bp, Artificial sequence
  Synthetic oligonucleotide.
atggagacacattctcaggtctttgtatacatgttgctgtggttgtctggtgttgaaggagacattgtgatgacccagtctcacaaattcatgtccacatcagtaggagacagggtcagcatcacctgcaaggccagtcaggatgtgggtactgctgtagcctggtatcaacagaaaccagggcaatctcctaaactactgatttactgggcatccacccggcacactggagtccctgatcgcttcacaggcagtggatctgggacagatttcactctcaccattaacaatgtgcagtctgaagacttggcagattatttctgtcagcaatatagcagcaatccgtacatgttcggaggggggaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 21 - protein, 234 aa, Artificial sequence
  Synthetic peptide.
METHSQVFVYMLLWLSGVEGDIVMTQSHKFMSTSVGDRVSITCKASQDVGTAVAWYQQKPGQSPKLLIYWASTRHTGVPDRFTGSGSGTDFTLTINNVQSEDLADYFCQQYSSNPYMFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - DNA, 1413 bp, Artificial sequence
  Synthetic oligonucleotide.
atgggatggagctggatctttctctttctcctgtcaggaactgcaggtgtcctctctgaggtccagctgcaacagtctggacctgagttggtgaagcctggggcttcagtgaagatatcctgcaagacttctggatacacattcactgaatacaccatgcactgggtgaggcagagccatggaaagagccttgagtggattggaggtattaatcctatcaatggtggtcctacctacaaccagaagttcaagggcaaggccacattgactgttgacaagtcctccagcacagcctacatggagctccgcagcctgacatctgaggactctgcagtctattactgtgcaagatgggactatggtagtcgagatgttatggactactggggtcaaggaacctcagtcaccgtctcctcagccaaaacgaagggcccatccgtcttccccctggcgccctgctccaggagcacctccgagagcacagccgccctgggctgcctggtcaaggactacttccccgaaccggtaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 23 - protein, 469 aa, Artificial sequence
  Synthetic peptide.
MGWSWIFLFLLSGTAGVLSEVQLQQSGPELVKPGASVKISCKTSGYTFTEYTMHWVRSHGKSLEWIGGINPINGGPTYNQKFKGKATLTVDKSSSTAYMELRSLTSEDSAVYYCARWDYGSRDVMDYWGQGTSVTVSSAKTKGPSVFPLAPCSRSTSESTAALGCLVKDYFPEPVPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 24 - DNA, 705 bp, Artificial sequence
  Synthetic oligonucleotide.
atggaatcacagactctggtcttcatatccatactgctctggttatatggtgctgatgggaacattgtaatgactcaatctcccaaatccatgtccatgtcagtaggggagagggtcaccttgagctgcaaggccagtgagaatgtgggaacttatgtatcctggtatcaacagagaccagaacagtctccaaaactgctgatatacggggcatccaaccggtacactggggtccccgatcgcttcacaggcagtggatctgcaacagatttcactctgaccatcagcagtgtgcaggctgaggaccttgcagattatcactgtggacagacttacagctatatattcacgttcggctcggggacaaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 25 - protein, 234 aa, Artificial sequence
  Synthetic peptide.
METHSQVFVYMLLWLSGVEGNIVMTQSPKSMSMSVGERVTLSCKASENVGTYVSWYQQRPEQSPKLLIYGASNRYTGVPDRFTGSGSATDFTLTISSVQAEDLADYHCGQTYSYIFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - DNA, 1404 bp, Artificial sequence
  Synthetic oligonucleotide.
atgggatggagccggatctttctcttcctcctgtcaataactgcaggtgtccattgccaggtccaggtgcagcagtcgggacctgagttggtgaagcctggggcctcagtgaagatttcctgcaaagcctctggcgacgcattcagtagttcttggatgaactgggtgaagcagaggcctggacagggtcttgagtggattggacggatttatcttggagatggagatattaattacaatgggaagttcaagggcagggccacactgactgcagacaaatcctccagcacagcctacatgcagctcagcagcctgacctctgtggactctgcggtctatttctgcgcgaggcagctcgggctatggtatgttatggactactggggtcaaggaacctcagtcaccgtctcctcagccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 27 - protein, 467 aa, Artificial sequence [sequence omitted]

SEQ ID NO: 28 - DNA, 705 bp, Artificial sequence
  Synthetic oligonucleotide.
atgagtgtgcccactcaggtcctggggttgctgctgctgtggcttacaggtgccagatgtgacatccagatggctcagtctccagcctccctatctgcatctgtgggagaaactgtcaccatcacatgtcgagcaagtgagaatatttacagttatttagcatggtatcagcagaaacagggaaaatctcctcagctcctggtctataatgcaaaaaccttagcagaaggtgtgccatcaaggttcagtggcagtggatcaggcacacagttttctctgaagatcaacagcctgcagcctgaagattttgggagttattactgtcaacatcattatggttttccgtggacgttcggtggaggcaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 29 - protein, 234 aa, Artificial sequence
  Syntehtic peptide.
MSVPTQVLGLLLLWLTGARCDIQMAQSPASLSASVGETVTITCRASENIYSYLAWYQQKQGKSPQLLVYNAKTLAEGVPSRFSGSGSGTQFSLKINSLQPEDFGSYYCQHHYGFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - DNA, 1395 bp, Artificial sequence
  Synthetic oligonucleotide.
atgaacttcgggctcagcttgattttccttgtcctcattttaaaaggtgtccagtgtgaggtgcagctggtggagtctgggggagacttagtgaagcctggagggtccctgaaactctcctgtgcagcctctggattcactttcagtagctatggcatgtcttgggttcgccagactccagacaagaggctggagtgggtcgcagtcattagtagtggtggaagttccaccttctatccagacagtgtgaaggggcgattcaccatctccagagacaatgccaagaacaccctgtacctgcaaatgagcagtctgaagtctgaggacacagccgtgtattactgttcaagaggaggttactactttgactactggggccaaggcaccactctcacagtctccgcagccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 31 - protein, 464 aa, Artificial sequence
  Syntehtic peptide.
MNFGLSLIFLVLILKGVQCEVQLVESGGDLVKPGGSLKLSCAASGFTFSSYGMSWVRQTPDKRLEWVAVISSGGSSTFYPDSVKGRFTISRDNAKNTLYLQMSSLKSEDTAVYYCSRGGYYFDYWGQGTTLTVSAAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 32 - DNA, 705 bp, Artificial sequence
  Synthetic oligonucleotide.
atgaggttccaggttcaggttctggggctccttctgctctggatatcaggtgcccagtgtgatgtccagataacccagtctccatcttatcttgctgcatctcctggagaaaccattactattaattgcagggcaagcaagaccattagcaaatatttagcctggtatcaagagaaacctgagaaaactgataagcttcttatctactctggatccactttgcaatctggaattccatcaaggttcagtggcagtggatctggtacagatttcactctcaccatcagtggcctggagcctgaagattttgcaatgtattactgtcaacagcataatgaatacccgtggacgttcggtggaggcaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 33 - protein, 234 aa, Artificial sequence
  Syntehtic peptide.
MRFQVQVLGLLLLWISGAQCDVQITQSPSYLAASPGETITINCRASKTISKYLAWYQEKPEKTDKLLIYSGSTLQSGIPSRFSGSGSGTDFTLTISGLEPEDFAMYYCQQHNEYPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - DNA, 1389 bp, Artificial sequence
  Syntehtic oligonucleotide.
atgggatggagctggatctttctctttctcctgtcaggaactgcaggtgtcctctctgaggtccagctgcaacagtctggacctgagctggtgaagcctggggcttcagtgaagatatcctgcaaggcttctggttactcattcactggctactacatgcactgggtgaagcaaagccatgtaaagagccttgagtggattggacgtattaatccttacaatggtgctactagctacaaccagaatttcaaggacaaggccagcttgactgtagataagtcctccagcacagcctacatggagctccacagcctgacatctgaggactctgcagtctattactgtgcaagagaggactacgtctactggggccaaggcaccactctcacagtctcctcagccaaaacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 35 - protein, 462 aa, Artificial sequence
  Syntehtic peptide.
MGWSWIFLFLLSGTAGVLSEVQLQQSGPELVKPGASVKISCKASGYSFTGYYMHWVKQSHVKSLEWIGRINPYNGATSYNQNFKDKASLTVDKSSSTAYMELHSLTSEDSAVYYCAREDYVYWGQGTTLTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 36 - DNA, 717 bp, Artificial sequence
  Synthetic oligonucleotide.
atgaagttgcctgttaggctgttggtgctgatgttctggattcctgcttccagcagtgatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcgcactcaagatcagtagagtggaggctgaggatctgggagtttatttctgctctcaaagtacacatgttccgtggacgttcggtggaggcaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 37 - protein, 238 aa, Artificial sequence
  Synthetic peptide.
MKLPVRLLVLMFWIPASSSDVVMTQTPLSLPVSLGDQASISCRSSQSLVHSNGNTYLHWYLQKPGQSPKLLIYKVSNRFSGVPDRFSGSGSGTDFALKISRVEAEDLGVYFCSQSTHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - DNA, 1410 bp, Artificial sequence
  Synthetic oligonucleotide.
atggaatggagttggatatttctctttcttctgtcaggaactgcaggtgtccactctgaggtccagctgcagcagtctggacctgagctggtaaagcctggggcttcagtgaagatgtcctgcaaggcttctggatacacattcactgactatgttttgcactgggtgaaacagaagcctgggcagggccttgagtggattggatatattaatccttacaatgatggtactaagtacaatgagaagttcaaaggcaaggccacactgacttcagacaaatcctccagcacagcctacatggagctcagcagcctgacctctgaggactctgcggtctattactgtgcaaggggctatccggcctactctgggtatgctatggactactggggtcaaggaacctcagtcaccgtctcctcagccaaaacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 39 - protein, 469 aa, Artificial sequence
  Syntehtic peptide.
MEWSWIFLFLLSGTAGVHSEVQLQQSGPELVKPGASVKMSCKASGYTFTDYVLHWVKQKPGQGLEWIGYINPYNDGTKYNEKFKGKATLTSDKSSSTAYMELSSLTSEDSAVYYCARGYPAYSGYAMDYWGQGTSVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 40 - DNA, 705 bp, Artificial sequence
  Syntehtic oligonucleotide.
atgatgtcctctgctcagttccttggtctcctgttgctctgttttcaaggtaccagatgtgatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccatcatggtaatacgcttccgtggacgttcggtggaggcaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 41 - protein, 234 aa, Artificial sequence
  Synthetic peptide.
MMSSAQFLGLLLLCFQGTRCDIQMTQTTSSLSASLGDRVTISCRASQDISNYLNWYQQKPDGTVKLLIYYTSRLHSGVPSRFSGSGSGTDYSLTISNLEQEDIATYFCHHGNTLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - DNA, 1404 bp, Artificial sequence
  Syntehtic oligonucleotide.
atgaacttggggctcagcttgattttccttgtccttgttttaaaaggtgtccagtgtgaagtgaagctggtggagtctgggggaggcttagtgcagcctggagggtccctgaaactctcctgtgcaacctctggattcactttcagtgactattacatgtattgggttcgccagactccagagaagaggctggagtgggtcgcatacattaattctggtggtggtagcacctattatccagacactgtaaagggccgattcaccatctccagagacaatgccaagaacaccctgtacctgcaaatgagccggctgaagtctgaggacacagccatgtattactgtgcaagacgggggttaccgttccatgctatggactattggggtcaaggaacctcagtcaccgtctcctcagccaaaacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 43 - protein, 467 aa, Artificial sequence
  Synthetic peptide.
MNLGLSLIFLVLVLKGVQCEVKLVESGGGLVQPGGSLKLSCATSGFTFSDYYMYWVRQTPEKRLEWVAYINSGGGSTYYPDTVKGRFTISRDNAKNTLYLQMSRLKSEDTAMYYCARRGLPFHAMDYWGQGTSVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 44 - DNA, 705 bp, Artificial sequence
  Synthetic oligonucleotide.
atgatgtcctctgctcagttccttggtctcctgttgctctgttttcaaggtaccagatgtgatatccagatgacacagactacatcctccctgtctgcctctctaggagacagagtcaccatcagttgcagtgcaagtcagggcattagcaattatttaaactggtatcagcagaaaccagatggaactgttaaactcctgatctattacacatcaattttacactcaggagtcccatcaaggttcagtggcagtgggtctgggacagattattctctcaccatcggcaacctggaacctgaagatattgccacttactattgtcagcagtttaataagcttcctccgacgttcggtggaggcaccaaactcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 45 - protein, 234 aa, Artificial sequence
  Synthetic peptide.
MMSSAQFLGLLLLCFQGTRCDIQMTQTTSSLSASLGDRVTISCSASQGISNYLNWYQQKPDGTVKLLIYYTSILHSGVPSRFSGSGSGTDYSLTIGNLEPEDIATYYCQQFNKLPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - DNA, 1405 bp, Artificial sequence
  Synthetic peptide.
atggattggctgtggaacttgctattcctgatggcagctgcccaaagtgcccaagcacagatccagttggtgcagtctggacctgagctgaagaagcctggagagacagtcaagatctcctgcaaggcttctgggtattccttcacaaactatggaatgaactgggtgaaacaggctccaggaaagggtttaaagtggatgggctggataaacacctacactggagagtcaacatatgctgatgacttcaagggacggtttgccttctctttggaaacctctgccagcactgcctatttgcagatcagtaacctcaaaaatgaggacatggctacatatttctgtgctagaggggactttaggtactactattttgactactggggccaaggcaccactctcacaggctcctcagccaaaacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctgat

SEQ ID NO: 47 - protein, 467 aa, Artificial sequence
  Synthetic peptide.
MDWLWNLLFLMAAAQSAQAQIQLVQSGPELKKPGETVKISCKASGYSFTNYGMNWVKQAPGKGLKWMGWINTYTGESTYADDFKGRFAFSLETSASTAYLQISNLKNEDMATYFCARGDFRYYYFDYWGQGTTLTGSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 48 - DNA, 702 bp, Artificial sequence
  Synthetic oligonucleotide.
atgagtgtgctcactcaggtcctggcgttgctgctgctgtggcttacaggtgccagatgtgacatccagatgactcagtctccagcctccctatctgcatctgtgggagaaactgtcaccatcacgtgtcgagcaagtgggaatattcacaattatttagcatggtatcagcagaaacagggaaaatctcctcagctcctggtctataatgcaaaaaccttggcagatggtgtgccatcaaggttcagtggcagtggatcaggaacacaatattctctcaagatcaacaccctgcagcctgaagattttgggagttattactgtcaacatttttgggattcttggacgttcggtggaggcaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 49 - protein, 233 aa, Artificial sequence
  Synthetic peptide.
MSVLTQVLALLLLWLTGARCDIQMTQSPASLSASVGETVTITCRASGNIHNYLAWYQQKQGKSPQLLVYNAKTLADGVPSRFSGSGSGTQYSLKINTLQPEDFGSYYCQHFWDSWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - DNA, 1398 bp, Artificial sequence
  Synthetic oligonucleotide.
atggaatggacctgggtctttctcttcctcctgtcagtaactgcaggtgtccactcccaggttcagctgcagcagtctggagctgagctgatgaagcctggggcctcagtgaagatatcctgcaaggctactggctacacattcagtagctactggatagagtgggtaaagcagaggcctggacatggccttgagtggattggagagattttacctggaagtggtaggactaacgacaatgagaagttcaagggcaaggccacattcactgcagatacatcctccaagaaagcctacatgcaactcagcagcctgacatctgaggactctgccgtctattattgtgcaagaaggggtggttactcctttgcttactggggccaagggactctggtcactgtctctgcagccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 51 - protein, 465 aa, Artificial sequence
  Synthetic peptide.
MEWTWVFLFLLSVTAGVHSQVQLQQSGAELMKPGASVKISCKATGYTFSSYWIEWVKQRPGHGLEWIGEILPGSGRTNDNEKFKGKATFTADTSSKKAYMQLSSLTSEDSAVYYCARRGGYSFAYWGQGTLVTVSAAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 52 - DNA, 1422 bp, Artificial sequence
  Synthetic oligonucleotide.
atgaccatgttctcactagctcttctcctcagtcttcttctcctctgtgtctctgattctagggcagaaacaactgtgacccagtctatgaccatgttctcactagctcttctcctcagtcttcttctcctctgtgtctctgattctagggcagaaacaactgtgacccagtctccagcatccctgtccatggctataggggaaaaagtcaccatcagatgcgtaaccagcactgatattgatgatgatgtgaactggtaccagcagaagccaggggaacctcctaaactccttatttcagaaggcaatactcttcgtcctggagtcccatcccgattctccagcagtggctatggtacagattttgtttttacaattgagaacatgctctcagaagatgttgcagattactactgtttgcaaagtggtaacttgccgtacacgttcggaggggggaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttagccagcatccctgtccatggctataggggaaaaagtcaccatcagatgcgtaaccagcactgatattgatgatgatgtgaactggtaccagcagaagccaggggaacctcctaaactccttatttcagaaggcaatactcttcgtcctggagtcccatcccgattctccagcagtggctatggtacagattttgtttttacaattgagaacatgctctcagaagatgttgcagattactactgtttgcaaagtggtaacttgccgtacacgttcggaggggggaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 53 - protein, 236 aa, Artificial sequence
  Synthetic peptide.
MTMFSLALLLSLLLLCVSDSRAETTVTQSPASLSMAIGEKVTIRCVTSTDIDDDVNWYQQKPGEPPKLLISEGNTLRPGVPSRFSSSGYGTDFVFTIENMLSEDVADYYCLQSGNLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - DNA, 1404 bp, Artificial sequence
  Synthetic oligonucleotide.
atggcttgggtgtggaccttgctattcctgatggcagctgcccaaagtgcccaagcacagatccagttggtgcagtctggacctgagctgaagaagcctggagagacagtcaagatctcctgcaaggcttctgggtataccttcacaaactatggaatgaactgggtgaagcaggctccaggaaagggtttaaagtgggtgggctggataaacaccttcactggagagccaacatatgttgatgacttcaagggacggtttgccttctctttggaaacctctgccagcactgcctatttgcagatcaacaacctcaaaaatgaggacacggctacatatttctgtgcaagagggaattttaggtactactactttgactactggggccaaggcaccactctcacagtctcctcagccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 55 - protein, 467 aa, Artificial sequence
  Synthetic peptide.
MAWVWTLLFLMAAAQSAQAQIQLVQSGPELKKPGETVKISCKASGYTFTNYGMNWVKQAPGKGLKWVGWINTFTGEPTYVDDFKGRFAFSLETSASTAYLQINNLKNEDTATYFCARGNFRYYYFDYWGQGTTLTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 56 - DNA, 705 bp, Artificial sequence
  Synthetic oligonucleotide.
atgagtgtgctcactcaggtcctggcgttgctgctgctgtggcttacaggtgccagatgtgacatccagatgactcagtccccagcctccctatctgcatctgtgggagaaactgtcaccatcacatgtcgaacaagtgggaatattcgcaattatttagcatggtatcagcagaaacagggaaaatctcctcaactcctggtctataatgcaaaaaccttagcagatggtgtgccatcaaggttcggtggcagtggatcaggaacacaatattctctcaagatcaacagcctgcagcctgaagattttgggaattattactgtcaacatttttggagtagtccgtacacgttcggaggggggaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 57 - protein, 234 aa, Artificial sequence
  Syntehtic peptide.
MSVLTQVLALLLLWLTGARCDIQMTQSPASLSASVGETVTITCRTSGNIRNYLAWYQQKQGKSPQLLVYNAKTLADGVPSRFGGSGSGTQYSLKINSLQPEDFGNYYCQHFWSSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - DNA, 2811 bp, Artificial sequence
  Synthetic oligonucleotide.
atgatgggatggagctatatcatcctctttttggtagcaacagctacagatgtccactcccaggtccaactgcagcagcctggggctgaactggtgaagcctggggcttcagtgaagctgtcctgcaaggcttctggctacaccttcaccagctactggatgcactgggtgaagcagaggcctggagaaggccttgagtggattggagagattaatcctagctacggtcgtactgactacaatgagaagttcaagaacaaggccacactgactgtagccaaatcctccagcacagcctacatgcaactcagcagcctgacatctgaggactctgcggtctattactgtgcaagaggagattactacggtagtagctcgtttgcttactggggccaagggactctggtcactgtctctgcagccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagcggatggagctatatcatcctctttttggtagcaacagctacagatgtccactcccaggtccaactgcagcagcctggggctgaactggtgaagcctggggcttcagtgaagctgtcctgcaaggcttctggctacaccttcaccagctactggatgcactgggtgaagcagaggcctggagaaggccttgagtggattggagagattaatcctagctacggtcgtactgactacaatgggaagttcaagaacaaggccacactgactgtagccaaatcctccagcacagcctacatgcaactcagcagcctgacatctgaggactctgcggtctattactgtgcaagaggagattactacggtagtagctcgtttgcttactggggccaagggactctggtcactgtctctgcagccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 59 - protein, 469 aa, Artificial sequence
  Synthetic peptide.
MMGWSYIILFLVATATDVHSQVQLQQPGAELVKPGASVKLSCKASGYTFTSYWMHWVKQRPGEGLEWIGEINPSYGRTDYNEKFKNKATLTVAKSSSTAYMQLSSLTSEDSAVYYCARGDYYGSSSFAYWGQGTLVTVSAAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 60 - DNA, 708 bp, Artificial sequence
  Synthetic oligonucleotide.
atggattttcaagtgcagattttcagcttcctgctaatgagtgcctcagtcataatgtccaggggacaaattgttctcacccagtctccagcactcatgtctgcatctccaggggagaaggtcaccatgacctgcagtgccagctcaaatataagttacatgtactggtaccagcagaagccaagatcctcccccaaaccctggatttatctcacatccaacctggcttctggagtccctgctcgcttcagtggcagtgggtctgggacctcttactctctcacaaccagcagcatggaggctgaagatgctgccacttattgctgccagcagtggagtagtaacccacccacgttcggtgctgggaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 61 - protein, 235 aa, Artificial sequence
  Synthetic peptide.
MDFQVQIFSFLLMSASVIMSRGQIVLTQSPALMSASPGEKVTMTCSASSNISYMYWYQQKPRSSPKPWIYLTSNLASGVPARFSGSGSGTSYSLTTSSMEAEDAATYCCQQWSSNPPTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - DNA, 708 bp, Artificial sequence
  Synthetic oligonucleotide.
atggattttcgagtgcagattttcagcttcctgctaatgagtgcctcagtcataatgtccaggggacaaattgttctcacccagtctccagcactcatgtctgcatctccaggggagaaggtcaccatgacctgcagtgccagctcaaatataagttacatgtactggtaccagcagaagccaagatcctcccccaaaccctggatttatctcacatccaacctggcttctggagtccctgctcgcttcagtggcagtgggtctgggacctcttactctctcacaatcagcagcatggaggctgaagatgctgccacttattactgccagcagtggagtagtaacccacccacgttcggtgctgggaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 63 - protein, 235 aa, Artificial sequence
  Synthetic peptide.
MDFRVQIFSFLLMSASVIMSRGQIVLTQSPALMSASPGEKVTMTCSASSNISYMYWYQQKPRSSPKPWIYLTSNLASGVPARFSGSGSGTSYSLTISSMEAEDAATYYCQQWSSNPPTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - DNA, 717 bp, Artificial sequence
  Synthetic oligonucleotide.
atggagacagacacactcctgctatgggtgctgctgctctgggttccaggttccacaggtgacattgtgctgatccaatctccagcttctttggctgtgtctctagggcagagggccaccatatcctgcagagccagtgaaagtgttgatagttatgtcaatagttttatgcactggtaccagcagaaaccaggacagccacccaaactcctcatctatcgtgtatccaacctagaatctgggatccctgccaggttcagtggcagtgggtctaggacagacttcaccctcaccattaatcctgtggaggctgatgatgttgcaacctattactgtcagcaaagtaatgaggatccattcacgttcggctcggggacaaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 65 - protein, 238 aa, Artificial sequence
  Synthetic peptide.
METDTLLLWVLLLWVPGSTGDIVLIQSPASLAVSLGQRATISCRASESVDSYVNSFMHWYQQKPGQPPKLLIYRVSNLESGIPARFSGSGSRTDFTLTINPVEADDVATYYCQQSNEDPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - DNA, 1398 bp, Artificial sequence
  Synthetic oligonucleotide.
atggaatgtaactggatacttccttttattctgtcggtaatttcaggggtctactcagaggttcagctccagcagtctgggactgtgctggcaaggcctggggcttccgtgaatatgtcctgtaaggctgctggctacagctttaccagttactgggtgtactgggtcaaacagaggcctggacagggtctggaatggattggtgctatttaccctaaaaatagtagaactagctacaaccagaagttccaggacaaggccacactgactgcagtcacatccgccagcactgcctacatggagctcagcagcctgacaaatgaggactctgcggtctattactgtacaagacctcactatgattcgtttggttactggggccaagggactctggtcactgtctctgcagccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 67 - protein, 465 aa, Artificial sequence
  Synthetic peptide.
MECNWILPFILSVISGVYSEVQLQQSGTVLARPGASVNMSCKAAGYSFTSYWVYWVKQRPGQGLEWIGAIYPKNSRTSYNQKFQDKATLTAVTSASTAYMELSSLTNEDSAVYYCTRPHYDSFGYWGQGTLVTVSAAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 68 - DNA, 717 bp, Artificial sequence
  Syntehtic oligonucleotide.
atggagacagacacactcctgctatgggtgctgctgctctgggttccaggttccacaggtgacattgtgctgacccaatctccagcttctttggctgtgtctctagggcagagggccaccatatcctgcagagccagtgaaagtgtagatagttatggcattagttttatgcactggtaccagcagaaaccaggacagccacccaaactcctcatctatcgtgcatccaaccaagaatctgggatccctgccaggttcagtggcagtgggtctaggacagacttcaccctcaccattaatcctgtggaggctgatgatgttgcaacctattactgtcagcaaagtaatgaggatccgctcacgttcggtgctgggaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 69 - protein, 238 aa, Artificial sequence
  Syntehtic peptide.
METDTLLLWVLLLWVPGSTGDIVLTQSPASLAVSLGQRATISCRASESVDSYGISFMHWYQQKPGQPPKLLIYRASNQESGIPARFSGSGSRTDFTLTINPVEADDVATYYCQQSNEDPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - DNA, 1422 bp, Artificial sequence
  Synthetic oligonucleotide.
atgaacaggcttacttcctcattgctgctgctgattgtccctgcatatgtcctgtcccaggttactctgaaagagtctggccctgggatattgcagccctcccagaccctcagtctgacttgttctttctctgggttttcactgagcacttctggtatgggtgtgagctggattcgtcagccttcaggaaagggtctggagtggctggcacacatttactgggatgatgacaagcgctataatccatccctgaagagccggctcacaatctttaaggatccctccagcaaccaggtattcctcaggatcaccagtgtggacactgcagatactgccacatactactgtgctcgaaactcccattactacggtagtacttacgggggatacttcgatgtctggggcgcagggaccacggtcaccgtctcctcagccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 71 - protein, 473 aa, Artificial sequence
  Synthetic peptide.
NRLTSSLLLLIVPAYVLSQQVTLKESGPGILQPSQTLSLTCSFSGFSLSTSGMGVSWIRQPSGKGLEWLAHIYWDDDKRYNPSLKSRLTIFKDPSSNQVFLRITSVDTADTATYYCARNSHYYGSTYGGYFDVWGAGTTVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 72 - DNA, 717 bp, Artificial sequence
  Synthetic oligonucleotide.
atggagacagacacactcctgctatgggtgctgctgctcggggttccaggttccacaggtaacattgtgctgacccagtctccaacttctttcactgtgtctcttgggcagagggccaccatatcctgcagagccagtgaaagtgttcatagttatggcaatagttttatgcactggtaccagcagaaaccagggcagccacccaaactcctcatctatcttgcatccaacgtagaatctggggtccctgccaggttcagtggtagtgggtccaggacagacttcaccctcaccattgatcctgtggaggctgatgatgctgcaacctattactgtcagcaaaatagtgaggatccgtggacgttcggtggaggcaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 73 - protein, 238 aa, Artificial sequence
  Synthetic peptide.
METDTLLLWVLLLGVPGSTGNIVLTQSPTSFTVSLGQRATISCRASESVHSYGNSFMHWYQQKPGQPPKLLIYLASNVESGVPARFSGSGSRTDFTLTIDPVEADDAATYYCQQNSEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - DNA, 1398 bp, Artificial sequence
  Synthetic oligonucleotide.
atggaatggacctgggtctttctcttcctcctgtcagtaactgcaggtgtccactcccaggttcagctgcagcagtctggaactgagctgatgaagcctggggcctcagtgaagatatcctgcaaggctactggctacacattcagtacctactggatagagtgggtaaagcagaggcctggacatggccttgagtggattggagagattttacctggaagtggtaggactaacgacaatgagaagttcaagggcaaggccacaatcactgcagatacatcctccaagaaagcctacatgcaactcagcagcctgacatctgaggactctgccgtctattactgtgcaagaaggggtggttactcctttgctttctggggccaagggactctggtctctgtctctgcagccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 75 - protein, 465 aa, Artificial sequence
  Synthetic peptide.
MEWTWVFLFLLSVTAGVHSQVQLQQSGTELMKPGASVKISCKATGYTFSTYWIEWVKQRPGHGLEWIGEILPGSGRTNDNEKFKGKATITADTSSKKAYMQLSSLTSEDSAVYYCARRGGYSFAFWGQGTLVSVSAAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 76 - DNA, 711 bp, Artificial sequence
  Synthetic oligonucleotide.
atgaccatgttctcactagctcttctcctcagtcttcttctcctctgtgtctctgattctagggcagaaacaactgtgacccagtctccagcatccctgtccatggctataggagaaaaagtcaccatcagatgcgtaaccagcactgatattgatgatgatgtgaactggtaccagcagaagccaggggaacctcctaagctccttatttcagaaggcaatactcttcgtgctggagtcccatcccgattctccagcagtggctatggtacagattttgtttttacaattgagaacatgctctcagaagatgttgcagattactactgtttgcaaagtggtaacttgccgtacacgttcggaggggggaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 77 - protein, 236 aa, Artificial sequence
  Synthetic peptide.
MTMFSLALLLSLLLLCVSDSRAETTVTQSPASLSMAIGEKVTIRCVTSTDIDDDVNWYQQKPGEPPKLLISEGNTLRAGVPSRFSSSGYGTDFVFTIENMLSEDVADYYCLQSGNLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - DNA, 1422 bp, Artificial sequence
  Synthetic oligonucleotide.
atgaacaggcttacttcctcattgctgctgctgattgtccctgcatatgtcctgtcccaggttactctgaaagagtctggccctgggatattgcagccctcccagaccctcagtctgacttgttctttctctgggttttcactgagcacttctggtatgggtctgagctggattcgtcagccttcaggaaagggtctggagtggctggcacacatttactgggatgatgacaagcgctataacccatccctgaagagccggctcacaatctccaaggatacctccagcaaccaggttttcctcaagatcaccattgtggacactgcagatgctgccacatactactgtgctcgaagctcccattactacggttatggctacgggggatacttcgatgtctggggcgcagggaccacggtcaccgtctcctcagccaaaacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 79 - protein, 473 aa, Artificial sequence
  Synthetic peptide.
MNRLTSSLLLLIVPAYVLSQVTLKESGPGILQPSQTLSLTCSFSGFSLSTSGMGLSWIRQPSGKGLEWLAHIYWDDDKRYNPSLKSRLTISKDTSSNQVFLKITIVDTADAATYYCARSSHYYGYGYGGYFDVWGAGTTVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 80 - DNA, 823 bp, Artificial sequence
  Synthetic oligonucleotide.
atggagacagacacactcctgctatgggtgctgctgctctgggttccaggttccacaggtaacattgtgctgacccaatctccagcttctttggctgtgtctctagggcagagggccaccatatcctgcagagccagtgaaagtattcatagttatggcaatagttttctgcactggtaccagcagaaaccaggacagccacccaaactcctcatctatcttgcatccaacctagaatctggggtccctgccaggttcagcggcagtgggtctaggacagacttcaccctcaccattgatcctgtggaggctgatgatgctgcaacctattactgtcagcaaaataatgaggatccgtggacgttcggtggaggcaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttaggcggccgcactagcgcgggccgcattcgaagagctcggtacccggggatcctctagagtcgacctgcaggcatgcaagctggccgcgactctagatcataatcagc

SEQ ID NO: 81 - protein, 238 aa, Artificial sequence
  Synthetic peptide.
METDTLLLWVLLLWVPGSTGNIVLTQSPASLAVSLGQRATISCRASESIHSYGNSFLHWYQQKPGQPPKLLIYLASNLESGVPARFSGSGSRTDFTLTIDPVEADDAATYYCQQNNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - DNA, 1404 bp, Artificial sequence
  Synthetic oligonucleotide.
atgaaatgcagctgggtcatcttcttcctgatggcagtggttacaggggtcaattcagaggttcagctgcagcagtctggggctgagcttgtgaggccaggggccttagtcaagttgtcctgcaaagcttctggcttcaacattaatgactactatatccactgggtgaagcagcggcctgaacagggcctggagcggattggatggattgatcctgacaatggtaatactatatatgacccgaagttccagggcaaggccagtataacagcagacacatcccccaacacagcctacctgcagctcagcagcctgacatctgaggacactgccgtctattactgtgctagaacccgatctcctatggttacgacggggtttgtttactggggccaagggactgtggtcactgtctctgcagccaaaacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacraagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 83 - protein, 467 aa, Artificial sequence
  Syntehtic peptide.
MKCSWVIFFLMAVVTGVNSEVQLQQSGAELVRPGALVKLSCKASGFNINDYYIHWVKQRPEQGLERIGWIDPDNGNTIYDPKFQGKASITADTSPNTAYLQLSSLTSEDTAVYYCARTRSPMVTTGFVYWGQGTVVTVSAAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKXKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 84 - DNA, 1425 bp, Artificial sequence
  Syntehtic oligonucleotide.
atggaaaggcactggatctttctcttcctgttttcagtaactgcaggtgtccactcccaggtccagcttcagcagtctggggctgagctggcaaaacctggggcctcagtgaagatgtcctgcaaggcttctggctacacctttactacctactggatgcactgggtaaaacagaggcctggacagggtctggaatggattggatacattaatcctatcactggttatactgagtacaatcagaagttcaaggacaaggccaccttgactgcagacaaatcctccagcacagcctacatgcaactgagcagcctgacatctgaggactctgcagtctattactgtgcaagagagggtttaagtgctatggactattggggtcagggaacctcagtcaccgtcacctcagccaaaacaacagccccatcggtctatccactggcccctgtgtgtggagatacaactggctcctcggtaactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaagctagctga

SEQ ID NO: 85 - protein, 474 aa, Artificial sequence
  Syntehtic peptide.
MERHWIFLFLFSVTAGVHSQVQLQQSGAELAKPGASVKMSCKASGYTFTTYWMHWVKQRPGQGLEWIGYINPITGYTEYNQKFKDKATLTADKSSSTAYMQLSSLTSEDSAVYYCAREGLSAMDYWGQGTSVTVTSAKTTAPSVYPLAPVCGDTTGSSVTLGCLVKGYFPEPVTLTWNSGSLSSGVHTFPAVLQSDLYTLSSSVTVTSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGKAS

SEQ ID NO: 86 - DNA, 717 bp, Artificial sequence
  Syntehtic oligonucleotide.
atgggcatcaagatggagtcacggattcaggcatttgtattcgtgtttctctggttgtctggtgttggcggagacattgtgatgacccagtctcacaaattcatgtccacatcagtaggagacagggtcagcgtcacctgcaaggccagtcaggatgtgacttctgctgtagcctggtatcaacaaaaaccagggcaatctcctaaactactgatttactgggcatccacccggcacactggagtccctgatcgcttcacaggcagtggatctgggacagattatactctcaccatcagcagtgggcaggctgaagacctggcactttattactgtcaccaatattatagcgctcctcggacgttcggtggaggcaccaagctggaagtcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccatcgtcaagagcttcaataggaatgagtgttag

SEQ ID NO: 87 - protein, 234 aa, Artificial sequence
  Synthetic peptide.
MESRIQAFVFVFLWLSGVGGDIVMTQSHKFMSTSVGDRVSVTCKASQDVTSAVAWYQQKPGQSPKLLIYWASTRHTGVPDRFTGSGSGTDYTLTISSGQAEDLALYYCHQYYSAPRTFGGGTKLEVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 88 - DNA, 1398 bp, Artificial sequence
  Syntehtic oligonucleotide.
atggaaaggcactggatctttctcttcctgttttcagtaactgcaggtgtccactcccaggtccagcttcagcagtctggggctgagctggcaaaacctggggcctcagtgaagatgtcctgcaaggcttctggctacacctttactacctactggatgcactgggtaaaacagaggcctggacagggtctggaatggattggatacattaatcctatcactggttatactgagtacaatcagaagttcaaggacaaggccaccttgactgcagacaaatcctccagcacagcctacatgcaactgagcagcctgacatctgaggactctgcagtctattactgtgcaagagagggtttaagtgctatggactattggggtcagggaacctcagtcaccgtcacctcagccaaaacaacgggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 89 - protein, 465 aa, Artificial sequence
  Synthetic peptide.
MERHWIFLFLFSVTAGVHSQVQLQQSGAELAKPGASVKMSCKASGYTFTTYWMHWVKQRPGQGLEWIGYINPITGYTEYNQKFKDKATLTADKSSSTAYMQLSSLTSEDSAVYYCAREGLSAMDYWGQGTSVTVTSAKTTGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 90 - DNA, 717 bp, Artificial sequence
  Synthetic oligonucleotide.
atgggcatcaagatggagtcacagattcaggcatttgtattcgtgtttctctggttgtctggtgttggcggagacattgtgatgacccagtctcacaaattcatgtccacatcagtaggagacagggtcagcgtcacctgcaaggccagtcaggatgtgacttctgctgtagcctggtatcaacaaaaaccagggcaatctcctaaactactgatttactgggcatccacccggcacactggagtccctgatcgcttcacaggcagtggatctgggacagattatactctcaccatcagcagtgggcaggctgaagacctggcactttattactgtcaccaatattatagcgctcctcggacgttcggtggaggcaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 91 - protein, 234 aa, Artificial sequence
  Syntehtic peptide.
MESQIQAFVFVFLWLSGVGGDIVMTQSHKFMSTSVGDRVSVTCKASQDVTSAVAWYQQKPGQSPKLLIYWASTRHTGVPDRFTGSGSGTDYTLTISSGQAEDLALYYCHQYYSAPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - DNA, 1401 bp, Artificial sequence
  Synthetic oligonucleotide.
atggctgtcctggcactactcctctgcctggtggctttcccaacttgtaccctgtcccaggtgcaactgaaggagtcaggacctggcctggtggcgccctcacagagcctgtccattacctgctctgtctctgggttctcattaagcaactatgatataagctggattcgccagccaccaggaaagggtctggagtggcttggagtaatgtggactggtggaggcgcaaattataattcagctttcatgtccagactgagcatcaacaaggacaactccaagagccaagtttttttaaaaatgaacaatctgcaaactgatgacacagccatttattactgtgtcagagatgcggtgaggtactggaacttcgatgtctggggcgcagggaccacggtcaccgtctcctcagccaaaacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 93 - protein, 466 aa, Artificial sequence
  Syntehtic peptide.
MAVLALLLCLVAFPTCTLSQVQLKESGPGLVAPSQSLSITCSVSGFSLSNYDISWIRQPPGKGLEWLGVMWTGGGANYNSAFMSRLSINKDNSKSQVFLKMNNLQTDDTAIYYCVRDAVRYWNFDVWGAGTTVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 94 - DNA, 708 bp, Artificial sequence
  Syntehtic oligonucleotide.
atggattttcaagcgcagattttcagcttcctgctaatcagtgcttcagtcataatgtccagaggacaaattgttctctcccagtcaccagcaatcctgtctgcatctccaggggagaaggtcacaatgacttgcagggccagctcaagtgtaagttacatacactggtaccagcagaagccaggatcctcccccaaaccctggatttatgccacatcccacctggcttctggagtccctgctcgcttcagtggcagtgggtctgggacctcttactctctcacaatcagcagagtggaggctgaagatactgccacttattactgccagcagtggagtagtaacccattcacgttcggctcggggacaaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 95 - protein, 235 aa, Artificial sequence
  Syntehtic peptide.
MDFQAQIFSFLLISASVIMSRGQIVLSQSPAILSASPGEKVTMTCRASSSVSYIHWYQQKPGSSPKPWIYATSHLASGVPARFSGSGSGTSYSLTISRVEAEDTATYYCQQWSSNPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - DNA, 1410 bp, Artificial sequence
  Syntehtic oligonucleotide.
atggaaaggcactggatctttctactcctgttgtcagtaactgcaggtgtccactcccaggtccagctgcagcagtctggggctgaactggcaagacctggggcctcagtgaagatgtcctgcaaggcttctggctacacctttactacctacactatgcactgggtaaaacagaggcctggacagggtctggaatggattggatacattaatcctagcagtggttatactaattacaatcagaagttcaaggacaaggccacattgactgcagacaaatcctccagcacagcctccatgcaactgagcagcctgacatctgaggactctgcagtctattactgtgcaagagagagggcggtattagtcccctatgctatggactactggggtcaaggaacctcagtcaccgtctcctcagccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 97 - protein, 469 aa, Artificial sequence
  Synthetic peptide.
MERHWIFLLLLSVTAGVHSQVQLQQSGAELARPGASVKMSCKASGYTFTTYTMHWVKQRPGQGLEWIGYINPSSGYTNYNQKFKDKATLTADKSSSTASMQLSSLTSEDSAVYYCARERAVLVPYAMDYWGQGTSVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 98 - DNA, 708 bp, Artificial sequence
  Synthetic oligonucleotide.
atgcattttcaagtgcagattttcagcttcctgctaatcagtgcctcagtcataatgtccagaggacaaattgttctcacccagtctccagcagtcatgtctgcatctccaggggagaaggtcaccataacctgcactgccagctcaagtttaagttacatgcactggttccagcagaagccaggcacttctcccaaactctggctttatagcacatccatcctggcttctggagtccctactcgcttcagtggcagtggatctgggacctcttactctctcacaatcagccgaatggaggctgaagatgctgccacttattactgccagcaaaggagtagttccccattcacgttcggctcggggacaaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 99 - protein, 235 aa, Artificial sequence
  Synthetic peptide.
MHFQVQIFSFLLISASVIMSRGQIVLTQSPAVMSASPGEKVTITCTASSSLSYMHWFQQKPGTSPKLWLYSTSILASGVPTRFSGSGSGTSYSLTISRMEAEDAATYYCQQRSSSPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - DNA, 1404 bp, Artificial sequence
  Syntehtic oligonucleotide.
atgggatggacctggatctttattttaatcctgtcagttactacaggtgtccactctgaggtccagctgcagcagtctggacctgagctggagaagcctggcgcttcagtgaagatatcctgcaaggcttctggttactccttcactggctacaacatgaactgggtgaaacagagcaatggaaagagccttgagtggattggaaatattgatccttactatggtgatactaactacaaccagaagttcaagggcaaggccacattgactgtagacaaatcctccagcacagcctacatgcacctcaagagcctgacatctgaggactctgcagtctattactgtgcaagaccctacggtagtgaggcctactttgcttactggggccaagggactctggtcactgtctctgcagccaaaacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 101 - protein, 467 aa, Artificial sequence
  Syntehtic peptide.
MGWTWIFILILSVTTGVHSEVQLQQSGPELEKPGASVKISCKASGYSFTGYNMNWVKQSNGKSLEWIGNIDPYYGDTNYNQKFKGKATLTVDKSSSTAYMHLKSLTSEDSAVYYCARPYGSEAYFAYWGQGTLVTVSAAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 102 - DNA, 705 bp, Artificial sequence
  Syntehtic oligonucleotide.
atggtgtccacttctcagctccttggacttttgcttttctggacttcagcctccagatgtgacattgtgatgactcagtctccagccaccctgtctgtgactccaggagatagagtctctctttcctgcagggccagccagagtattagcgactacttacactggtatcaacaaaaatcacatgagtctccaaggcttctcatcaaatatgctgcccaatccatctctgggatcccctccaggttcagtggcagtggatcagggtcagatttcactctcagtatcaacggtgtggaacctgaagatgttggagtgtattactgtcaaaatggtcacagctttccgtacacgttcggaggggggaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 103 - protein, 214 aa, Artificial sequence
  Syntehtic peptide.
DIVMTQSPATLSVTPGDRVSLSCRASQSISDYLHWYQQKSHESPRLLIKYAAQSISGIPSRFSGSGSGSDFTLSINGVEPEDVGVYYCQNGHSFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - DNA, 1395 bp, Artificial sequence
  Syntehtic oligonucleotide.
atggaatggaggatctttctcttcatcctgtcaggaactgcaggtgtccactcccaggttcagctgcggcagtctggacctgagctggtgaagcctggggcttcagtgaagatgtcctgcaaggcttctggatacacatttactgactatgttataagttgggtgaagcagagaactggacagggccttgagtggattggagatatttatcctggaagtggttattctttctacaatgagaacttcaagggcaaggccacactgactgcagacaaatcctccaccacagcctacatgcagctcagcagcctgacatctgaggactctgcggtctatttctgtgcaacctactataactacccttttgcttactggggccaagggactctggtcactgtctctgcagccaaaacaacgggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 105 - protein, 446 aa, Artificial sequence
  Syntehtic peptide.
QVQLRQSGPELVKPGASVKMSCKASGYTFTDYVISWVKQRTGQGLEWIGDIYPGSGYSFYNENFKGKATLTADKSSTTAYMQLSSLTSEDSAVYFCATYYNYPFAYWGQGTLVTVSAAKTTGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 106 - DNA, 717 bp, Artificial sequence
  Syntehtic oligonucleotide.
atgaagttgcctgttaggctgttggtgctgatgttctggattcctgcttccagcagtgatgttgtgatgacccaaactccactctccctgcctgtccgtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacaaatttcacactcaagatcagcagagtggaggctgaggatctgggactttatttctgctctcaaagtacacatgttccgtacacgttcggaggggggaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 107 - protein, 219 aa, Artificial sequence
  Syntehtic peptide.
DVVMTQTPLSLPVRLGDQASISCRSSQSLVHSNGNTYLHWYLQKPGQSPKLLIYKVSNRFSGVPDRFSGSGSGTNFTLKISRVEAEDLGLYFCSQSTHVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - DNA, 1410 bp, Artificial sequence
  Syntehtic oligonucleotide.
atgacattgaacatgctgttggggctgaggtgggttttctttgttgttttttatcaaggtgtgcattgtgaggtgcagcttgttgagtctggtggaggattggtgcagcctaaagggtcattgaaactctcatgtgcagcctctggattaaccttcaatatctacgccatgaactgggtccgccaggctccaggaaagggtttggaatgggttgctcgcataagaaataaaagtaataattatgcaacatattatgccgattcagtgaaagacaggttcaccatctccagagatgattcacaaagcttgctctatctgcaaatgaacaacttgaaaactgaggacacagccatgtattactgtgtgggacgggactggtttgattactggggccaagggactctggtcactgtctctgcagccaaaacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 109 - protein, 469 aa, Artificial sequence
  Syntehtic peptide.
MTLNMLLGLRWVFFVVFYQGVHCEVQLVESGGGLVQPKGSLKLSCAASGLTFNIYAMNWVRQAPGKGLEWVARIRNKSNNYATYYADSVKDRFTISRDDSQSLLYLQMNNLKTEDTAMYYCVGRDWFDYWGQGTLVTVSAAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 110 - DNA, 708 bp, Artificial sequence
  Syntehtic peptide.
atggcctggatttcacttatactctctctcctggctctcagctcaggggccatttcccaggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagtttcaggtgttcctgccagattctcaggctccctgattggagacaaggctgccctcaccatcacaggggcacagactgaggatgaggcaatatatttctgtgctctatggtacagcaaccattgggtgttcggtggaggaaccaaactcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 111 - protein, 235 aa, Artificial sequence
  Syntehtic peptide.
MAWISLILSLLALSSGAISQAVVTQESALTTSPGETVTLTCRSSTGAVTTSNYANWVQEKPDHLFTGLIGGTNNRVSGVPARFSGSLIGDKAALTITGAQTEDEAIYFCALWYSNHWVFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - DNA, 1386 bp, Artificial sequence
  Syntehtic oligonucleotide.
atggaatggacctgggtctttctcttcctcctgtcagtaactgcaggtgtccactcccaggttcagctgcagcagtctggagctgagctgatgaagcctggggcctcagtgaagatatcctgcaaggctactggctacacattcggtagctactggatagagtgggtaaagcagaggcctggacatggccttgagtggattggagagattttacctggaagtggtaatactaactacaatgagaacttcaagggcaaggccacattcactgcagatacatcctccaacacagcctacatgcaactcaccagtctgacatctgaggactctgccgtctattactgtgctagggcggggatttattggggccaagggactctggtcactgtctctgcagccaaaacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 113 - protein, 461 aa, Artificial sequence
  Synthetic peptide.
MEWTWVFLFLLSVTAGVHSQVQLQQSGAELMKPGASVKISCKATGYTFGSYWIEWVKQRPGHGLEWIGEILPGSGNTNYNENFKGKATFTADTSSNTAYMQLTSLTSEDSAVYYCARAGIYWGQGTLVTVSAAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 114 - DNA, 717 bp, Artificial sequence
  Synthetic oligonucleotide.
atggagaaagacacactcctgctatgggtcctgcttctctgggttccaggttccacaggtgacattgtgctgacccaatctccagcttttttggctgtgtctctagggcagagggccaccatctcctgcagagccagcgaaagtgttgataattatggcattagttttatgaactggttccaacagaaaccaggacagccacccaaactcctcatctatgttgcatccaagcaaggatccggggtccctgccaggtttagtggcagtgggtctgggacagacttcagcctcaacatccatcctatggaggaggatgatactgcaatgtatttctgtcagcaaagtaaggaggttcctcggacgttcggtggaggcaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 115 - protein, 238 aa, Artificial sequence
  Synthetic peptide.
MEKDTLLLWVLLLWVPGSTGDIVLTQSPAFLAVSLGQRATISCRASESVDNYGISFMNWFQQKPGQPPKLLIYVASKQGSGVPARFSGSGSGTDFSLNIHPMEEDDTAMYFCQQSKEVPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - DNA, 1392 bp, Artificial sequence
  Synthetic oligonucleotide.
atggaatgtaactggatacttccttttattctgtcggtaacttcaggggtctactcagaggttcagctccagcagtctgggactgtgctggcaaggcctggggcttcagtgaagatgtcctgcaaggcttctggctacacctttaccagctactggatgcactgggtaaaacagaggcctggacagggtctggaatggattggcgctatttatcctggaaatagtgatactacctacaaccagaagttcaagggcaaggccaaactgactgcagtcacatccaccagcactgcctacatggagctcagcagcctgacaaatgaggactctgcggtctattactgtacacctacttactactttgactactggggccaaggcacctctctcacagtctcctcagccaaaacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 117 - protein, 463 aa, Artificial sequence
  Synthetic peptide.
MECNWILPFILSVTSGVYSEVQLQQSGTVLARPGASVKMSCKASGYTFTSYWMHWVKQRPGQGLEWIGAIYPGNSDTTYNQKFKGKAKLTAVTSTSTAYMELSSLTNEDSAVYYCTPTYYFDYWGQGTSLTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 118 - DNA, 717 bp, Artificial sequence
  Syntehtic oligonucleotide.
atgagtcctgcccaattcctgtttctgttagtgctctggattcgggaaaccaacggtgatgttgtgatgacccagactccactcactttgtcggttaccattggacaaccagcctccatctcttgcaagtcaagtcagagcctcttagatagtgatggaaagacatatttgaattggttcttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttccgtggacgttcggtggaggcaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 119 - protein, 238 aa, Artificial sequence
  Synthetic peptide.
MSPAQFLFLLVLWIRETNGDVVMTQTPLTLSVTIGQPASISCKSSQSLLDSDGKTYLNWFLQRPGQSPKRLIYLVSKLDSGVPDRFTGSGSGTDFTLKISRVEAEDLGVYYCWQGTHFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - DNA, 1415 bp, Artificial sequence
  Synthetic oligonucleotide.
atgggagggatctggatctttctcttcctcctgtcaggaactgcaggtgcccactctgagatccagctgcagcagactggacctgagctggtgaagcctggggcttcagtgaagatatcctgcaaggcttctggttatccattcactgactacatcatggtctgggtgaagcagagccatggaaagagccttgagtggattggaaatattagtccttactatggtactactaactacaatctgaagttcaagggcaaggccacattgactgtagacaaatcttccagcacagcctacatgcagctcaacagtctgacatctgaggactctgcagtctattactgtgcaagatcccctaactgggacggggcctggtttgctcactggggccaaggggctctggtcactgtctctgcagccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctgattaattaa

SEQ ID NO: 121 - protein, 468 aa, Artificial sequence
  Syntehtic peptide.
MGGIWIFLFLLSGTAGAHSEIQLQQTGPELVKPGASVKISCKASGYPFTDYIMVWVKQSHGKSLEWIGNISPYYGTTNYNLKFKGKATLTVDKSSSTAYMQLNSLTSEDSAVYYCARSPNWDGAWFAHWGQGALVTVSAAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 122 - DNA, 717 bp, Artificial sequence
  Synthetic oligonucleotide.
atggagacagacacaatcctgctatgggtgctgctgctctgggttccaggctccactggtgacattgtgctgacccaatctccagcttctttggctgtgtctctagggcagagggccaccatctcctgcaaggccagccaaagtgttgattatgatggtgatagttatatgaactggttccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatccattcacgttcggctcggggacaaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 123 - protein, 238 aa, Artificial sequence
  Synthetic peptide.
METDTILLWVLLLWVPGSTGDIVLTQSPASLAVSLGQRATISCKASQSVDYDGDSYMNWFQQKPGQPPKLLIYAASNLESGIPARFSGSGSGTDFTLNIHPVEEEDAATYYCQQSNEDPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - DNA, 1403 bp, Artificial sequence
  Syntehtic oligonucleotide.
atggctgtcctggggctgcttctctgcctggtgacgttcccaagctgtgtcctgtcccaggtgcagctgaaggagtcaggacctggcctggtggcaccctcacagagcctgtccatcacatgcactgtctctgggttctcattatccagatatagtgtattttgggttcgccagcctccaggaaagggtctggagtggctgggattgatatggggtggtggaagcacagactataattcagctctcaaatccagactgagcatcagcaaggacaactccaagagccaagttttcttaaaaatgaacagtctgcaaactgatgacacagccatgtactactgtgccagaatctactttgattacgacggggctatggactactggggtcaaggaacctcagtcaccgtctcctcagccaaaacaacgggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctg

SEQ ID NO: 125 - protein, 467 aa, Artificial sequence
  Syntehtic peptide.
MAVLGLLLCLVTFPSCVLSQVQLKESGPGLVAPSQSLSITCTVSGFSLSRYSVFWVRQPPGKGLEWLGLIWGGGSTDYNSALKSRLSISKDNSKSQVFLKMNSLQTDDTAMYYCARIYFDYDGAMDYWGQGTSVTVSSAKTTGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 126 - DNA, 732 bp, Artificial sequence
  Syntehtic oligonucleotide.
atgcatcgcaccagcatgggcatcaagatggagtcacggattcaggcatttgtattcgtgtttctctggttgtctggtgttggcggagacattgtgatgacccagtctcacaaattcatgtccacatcagtaggagacagggtcagcgtcacctgcaaggccagtcaggatgtgacttctgctgtagcctggtatcaacaaaaaccagggcaatctcctaaactactgatttactgggcatccacccggcacactggagtccctgatcgcttcacaggcagtggatctgggacagattatactctcaccatcagcagtgggcaggctgaagacctggcactttattactgtcaccaatattatagcgctcctcggacgttcggtggaggcaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 127 - protein, 243 aa, Artificial sequence
  Synthetic peptide.
MHRTSMGIKMESRIQAFVFVFLWLSGVGGDIVMTQSHKFMSTSVGDRVSVTCKASQDVTSAVAWYQQKPGQSPKLLIYWASTRHTGVPDRFTGSGSGTDYTLTISSGQAEDLALYYCHQYYSAPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - DNA, 1404 bp, Artificial sequence
  Synthetic oligonucleotide.
atggaatggaactgggtcgttctcttcctcctgtcattaactgcaggtgtctatgcccagggtcagatgcagcagtctggagctgagctggtgaagcctggggcttcagtgaagctgtcctgcaagacttctggcttcaccttcagcagtaactatataagttggttgaagcaaaagcctggacagagtcttgagtggattgcatggatttatgctggaactggtggtattacctataatcagaagttcagaggcagggcccaactgactgtagacacatcctccagcacagcctacatgcagttcagcagcctgacaactgatgactctgccatctattactgtgcaagacacgtgaggggttaccatcctatggactactggggtcaaggaacctcagtcaccgtctcctcagccaaaacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 129 - protein, 467 aa, Artificial sequence
  Synthetic peptide.
MEWNWVVLFLLSLTAGVYAQGQMQQSGAELVKPGASVKLSCKTSGFTFSSNYISWLKQKPGQSLEWIAWIYAGTGGITYNQKFRGRAQLTVDTSSSTAYMQFSSLTTDDSAIYYCARHVRGYHPMDYWGQGTSVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 130 - DNA, 705 bp, Artificial sequence
  Synthetic oligonucleotide.
atggagtcacagactcaggtctttgtatacatgttgctgtggttgtctggtgttgatggagacattgtgatgacccagtctcaaaaattcatgtccgcatcagtaggggacagggtcagcgtcacctgcagggccagtcagaatgtggttactaatgtaggctggtatcaacagaaaccagggcaatctcctaaagtactgatttactcggcatccttccggtacagtggagtccctgatcgcttcacaggcagtggatctgggacagatttcactctcaccatcaccaatgtgcagtctgaagacttggcagagtatttctgtcagcaatataacaactatccgtacacgttcggaggggggaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 131 - protein, 234 aa, Artificial sequence
  Synthetic peptide.
MESQTQVFVYMLLWLSGVDGDIVMTQSQKFMSASVGDRVSVTCRASQNVVTNVGWYQQKPGQSPKVLIYSASFRYSGVPDRFTGSGSGTDFTLTITNVQSEDLAEYFCQQYNNYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - DNA, 1407 bp, Artificial sequence
  Synthetic oligonucleotide.
atgggatggagctatatcatcctctttttggtagcaacagctacagatgtccactcccaggtccaactgcagcagcctggggctgaactggtgaagcctggggcttcagtgaagctgtcctgcaaggcttctggctacaccttcaccagctactggatgcactgggtgaagcagaggcctggagaaggccttgagtggattggagagattaatcctagctacggtcgtactgactacaatgggaagttcaagaacaaggccacactgactgtagccaaatcctccagcacagcctacatgcaactcagcagcctgacatctgaggactctgcggtctattactgtgcaagaggagattactacggtagtagctcgtttgcttactggggccaagggactctggtcactgtctctgcagccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 133 - protein, 468 aa, Artificial sequence
  Syntehtic peptide.
MGWSYIILFLVATATDVHSQVQLQQPGAELVKPGASVKLSCKASGYTFTSYWMHWVKQRPGEGLEWIGEINPSYGRTDYNGKFKNKATLTVAKSSSTAYMQLSSLTSEDSAVYYCARGDYYGSSSFAYWGQGTLVTVSAAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 134 - DNA, 705 bp, Artificial sequence
  Synthetic oligonucleotide.
atggagtcacagactcaggtctttgtatacatgttgctgtggttgtctggtgttgatggagacattgtgatgacccagtctcaaaaattcatgtccacatcattaggagacagggtcagcgtcacctgcaaggccagtcagaatgtgggtactaatgtagcctggtatcaacagaaaccagggcactctcctaaagcactgatttactcggcatcctaccggtacagtggagtccctgatcgcttcacaggcagtggatctgggacagatttcactctcaccatcagcaatgtgcagtctgaagacttggcagagtttttctgtcagcaatataacaactatccgtacacgttcggaggggggaccacgctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 135 - protein, 234 aa, Artificial sequence
  Syntehtic peptide.
MESQTQVFVYMLLWLSGVDGDIVMTQSQKFMSTSLGDRVSVTCKASQNVGTNVAWYQQKPGHSPKALIYSASYRYSGVPDRFTGSGSGTDFTLTISNVQSEDLAEFFCQQYNNYPYTFGGGTTLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 32 aa, Artificial sequence
  Syntehtic peptide.
VGFPVTPQVPLRPMTYKAAVDLSHFLKEKGGL

SEQ ID NO: 137 - protein, 30 aa, Artificial sequence
  Synthetic peptide.
HTQGYFPDWQNYTPGPGVRYPLTFGWLYKL

SEQ ID NO: 138 - protein, 19 aa, Artificial sequence
  Synthetic peptide.
EKIRLRPGGKKKYKLKHIV

SEQ ID NO: 139 - protein, 32 aa, Artificial sequence
  Synthetic peptide.
NPPIPVGEIYKRWIILGLNKIVRMYSPTSILD

SEQ ID NO: 140 - protein, 31 aa, Artificial sequence
  Synthetic peptide.
AIFQSSMTKILEPFRKQNPDIVIYQYMDDLY

SEQ ID NO: 141 - protein, 60 aa, Artificial sequence
  Syntehtic peptide.
MWVPVVFLTLSVTWIGAAPLILSRIVGGWECEKHSQPWQVLVASRGRAVCGGVLVHPQWV

SEQ ID NO: 142 - protein, 60 aa, Artificial sequence
  Syntehtic peptide.
LTAAHCIRNKSVILLGRHSLFHPEDTGQVFQVSHSFPHPLYDMSLLKNRFLRPGDDSSHD

SEQ ID NO: 143 - protein, 60 aa, Artificial sequence
  Syntehtic peptide.
LMLLRLSEPAELTDAVKVMDLPTQEPALGTTCYASGWGSIEPEEFLTPKKLQCVDLHVIS

SEQ ID NO: 144 - protein, 60 aa, Artificial sequence
  Synthetic peptide.
NDVCAQVHPQKVTKFMLCAGRWTGGKSTCSGDSGGPLVCNGVLQGITSWGSEPCALPERP

SEQ ID NO: 145 - protein, 21 aa, Artificial sequence
  Synthetic peptide.
SLYTKVVHYRKWIKDTIVANP

SEQ ID NO: 146 - protein, 9 aa, Artificial sequence
  Synthetic peptide.
IMDQVPFSV

SEQ ID NO: 147 - protein, 9 aa, Artificial sequence
  Syntehtic peptide.
ITDQVPFSV

SEQ ID NO: 148 - protein, 9 aa, Artificial sequence
  Synthetic peptide.
YLEPGPVTV

SEQ ID NO: 149 - protein, 9 aa, Artificial sequence
  Synthetic peptide.
YLEPGPVTA

SEQ ID NO: 150 - protein, 9 aa, Artificial sequence
  Synthetic peptide.
KTWGQYWQV

SEQ ID NO: 151 - protein, 230 aa, Artificial sequence
  Synthetic peptide.
DTTEPATPTTPVTTPTTTKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSQSYVPLAHSSSAFTITDQVPFSVSVSQLRALDGGNKHFLRNQ

SEQ ID NO: 152 - protein, 73 aa, Artificial sequence
  Synthetic peptide.
PLTFALQLHDPSGYLAEADLSYTWDFGDSSGTLISRAXVVTHTYLEPGPVTAQVVLQAAIPLTSCGSSPVPAS

SEQ ID NO: 153 - protein, 109 aa, Artificial sequence
  Synthetic peptide.
GTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAA

SEQ ID NO: 154 - protein, 75 aa, Artificial sequence
  Syntehtic peptide.
QVTTTEWVETTARELPIPEPEGPDASSIMSTESITGSLGPLLDGTATLRLVKRQVPLDCVLYRYGSFSVTLDIVQ

SEQ ID NO: 155 - protein, 109 aa, Artificial sequence
  Synthetic peptide.
GIESAEILQAVPSGEGDAFELTVSCQGGLPKEACMEISSPGCQPPAQRLCQPVLPSPACQLVLHQILKGGSGTYCLNVSLADTNSLAVVSTQLIVPGILLTGQEAGLGQ

SEQ ID NO: 156 - protein, 50 aa, Artificial sequence
  Syntehtic peptide.
MEMKILRALNFGLGRPLPLHFLRRASKIGEVDVEQHTLAKYLMELTMLDY

SEQ ID NO: 157 - protein, 36 aa, Artificial sequence
  Syntehtic peptide.
DWLVQVQMKFRLLQETMYMTVSIIDRFMQNNCVPKK

SEQ ID NO: 158 - protein, 48 aa, Artificial sequence
  Syntehtic peptide.
MEHQLLCCEVETIRRAYPDANLLNDRVLRAMLKAEETCAPSVSYFKCV

SEQ ID NO: 159 - protein, 95 aa, Artificial sequence
  Syntehtic peptide.
QKEVLPSMRKIVATWMLEVCEEQKCEEEVFPLAMNYLDRFLSLEPVKKSRLQLLGATCMFVASKMKETIPLTAEKLCIYTDNSIRPEELLQMELL

SEQ ID NO: 160 - protein, 60 aa, Artificial sequence
  Syntehtic peptide.
LVNKLKWNLAAMTPHDFIEHFLSKMPEAEENKQIIRKHAQTFVALCATDVKFISNPPSMV

SEQ ID NO: 161 - protein, 91 aa, Artificial sequence
  Synthetic peptide.
AGSVVAAVQGLNLRSPNNFLSYYRLTRFLSRVIKCDPDCLRACQEQIEALLESSLRQAQQNMDPKAAEEEEEEEEEVDLACTPTDVRDVDI

(19) United States
(12) Patent Application Publication
(10) Pub. No.: US 2012/0039916 A1
(43) Pub. Date: Feb. 16, 2012

(54) NOVEL VACCINE ADJUVANTS BASED ON TARGETING ADJUVANTS TO ANTIBODIES DIRECTLY TO ANTIGEN-PRESENTING CELLS

(75) Inventors: Gerard Zurawski, Midlothian, TX (US); Jacques F. Banchereau, Montclair, NJ (US); Anne-Laure Flamar, Dallas, TX (US)

(73) Assignee: BAYLOR RESEARCH INSTITUTE, Dallas, TX (US)

(21) Appl. No.: 13/208,993

(22) Filed: Aug. 12, 2011

Related U.S. Application Data
(60) Provisional application No. 61/373,763, filed on Aug. 13, 2010.

(51) Int. Cl. A61K 39/395; A61P 31/12; A61P 31/04; C12N 5/071; A61P 33/00; A61P 33/02; A61P 37/04; A61P 37/08; C07K 17/00; A61P 31/10

(52) U.S. Cl. 424/182.1; 530/391.7; 435/375

(57) ABSTRACT

Compositions and methods for enhancing an immune response with an adjuvant composition comprising: an anti-dendritic cell (DC)-specific antibody or fragment thereof conjugated to at least a portion of a TLR agonist; and a pharmaceutically acceptable carrier are disclosed herein. The conjugate and agonist are each comprised in an amount such that, in combination with the other, are effective to produce the immune response in a human or animal subject in need of immunostimulation.

### CROSS-REFERENCE TO RELATED APPLICATIONS

This application is a non-provisional application of U.S. provisional patent application No. 61/373,763 filed on Aug. 13, 2010 and entitled “Novel Vaccine Adjuvants Based on Targeting Adjuvants to Antibodies Directly to Antigen-Presenting Cells” the entire contents of which is incorporated herein by reference.

### STATEMENT OF FEDERALLY FUNDED RESEARCH

This invention was made with U.S. Government support under Contract Nos. 1 U19 AI057234 awarded by the National Institutes of Health (NIH)/National Institute for Allergy and Infectious Diseases (NIAID). The government has certain rights in this invention.

### TECHNICAL FIELD OF THE INVENTION

The present invention relates in general to the dendritic cell (DC)-targeting vaccines, and more particularly, to the enhancing vaccine efficacy by directly linking adjuvants (e.g., TLR ligands) directly to DC-targeting vaccines.

### REFERENCE TO A SEQUENCE LISTING

The present application includes a Sequence Listing filed separately as required by 37 CFR 1.821-1.825.

### BACKGROUND OF THE INVENTION

Without limiting the scope of the invention, its background is described in connection with novel adjuvants, dendritic cell (DC) vaccines, and strategies for enhancing immune responses to DC vaccines.

U.S. Patent Application Publication No. 20090004194 (Kedl, 2007) relates to novel protein and DNA conjugates which promote antigen specific cellular immunity. The use of these polypeptide conjugates and DNA conjugates as immune adjuvants for treating various chronic diseases including cancer, infectious diseases, autoimmune diseases, allergic and inflammatory diseases. The Kedl invention discloses fusion proteins and DNA conjugates containing a TLR/CD40/agonist and optional antigen combination. The use of these protein and DNA conjugates as immune adjuvants and as vaccines for treatment of various chronic diseases is also taught.

U.S. Patent Application Publication No. 20080220011 (Steven, 2008) provides a fusion protein comprising a flagellin adjuvant and a tumor antigen. Also provided are compositions comprising a flagellin adjuvant and a tumor antigen. The invention further provides pharmaceutical formulations and methods for inducing an immune response against a tumor antigen and methods of treating a tumor in a subject.

U.S. Patent Application Publication No. 20080248068 (Ljunggren et al. 2008) is directed to flagellin and its use as an adjuvant for vaccination. The invention can be used in vaccine formulations to improve immunity against any other antigen administered at the same localization. The antigen can be administered in the same construct as Flagellin or in any other formulation given at the same localization. As an alternative flagellin can be used to stimulate immunity against antigens expressed at a specific location. Flagellin can also be used to induce local inflammation with the purpose of creating a model for inflammation.

U.S. Pat. No. 7,404,963 issued to Sotomayor and Suarez (2008) provides adjuvants, vaccines, and related methods that are useful in eliciting immune responses, particularly immune responses against tumor antigens. According to the Sotomayor invention flagellin is capable of inhibiting tolerance when it is administered in conjunction with a tolerogenic antigen. This effect is likely mediated by the ability of flagellin to induce IL-12 while keeping IL-10 levels low. Furthermore, flagellin can be provided in an extended-releasing manner by using a flagellin-expressing cell. Preferably, the flagellin-expressing cell is treated such that it is no longer capable of replicating, yet retaining the ability to express flagellin, such as by lethal irradiation.

### SUMMARY OF THE INVENTION

The present invention describes compositions and methods for making novel vaccine adjuvants based on targeting adjuvants with antibodies directly to antigen-presenting cells. The present invention was found to enhance vaccine efficacy by linking adjuvants (e.g., TLR ligands) directly to DC-targeting vaccines. As shown herein, the compositions and methods of the present invention are broadly applicable to all DC-targeting vaccines and extensible to making adjuvants with unexpected novel properties.

The present invention in one embodiment discloses an adjuvant composition comprising an anti-dendritic cell (DC)-specific antibody or binding fragment thereof conjugated to a TLR agonist; and at least one antigen, wherein the antigen and the agonist are effective to produce an immune response in a human or animal subject in need of immunostimulation.

The DC-specific antibody or fragment described hereinabove is selected from an anti-DCIR, MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, or ASPGR. In one aspect, the composition described above further comprises antigenic peptides selected from human immunodeficiency virus (HIV) antigens and gene products selected from the group consisting of gag, pol, and env genes, the Nef protein, reverse transcriptase, string of HIV peptides (Hipo5), PSA (KLQCVDLHV)-tetramer, a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide (GILGFVFTL) tetramer, and Avian Flu (HA5-1), dockerin domain from C. thermocellum, measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens or combinations and modifications thereof. In another aspect the composition further comprises antigenic peptides selected from one or more bacterial antigens, wherein the bacterial antigens comprise antigens derived from Bacillus, Escherichia, Listeria, Neisseria, Nocardia, Salmonella, Staphylococcus, Streptococcus, or combinations and modifications thereof.

In yet another aspect the composition further comprises antigenic peptides selected from cancer peptides selected from tumor associated antigens comprising antigens from leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia. In a specific aspect the tumor associated antigens are selected from CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67.

In other aspects related to the composition of the present invention DC-specific antibody is humanized. In another aspect the TLR agonist comprises at least one of a flagellin from Salmonella enterica, a flagellin from Vibrio cholerae, any other TLR5 agonist, a TLR7 agonist, a TLR9 agonist, or any combinations or modifications thereof. In yet another aspect the antigen is conjugated to the antibody and TLR agonist. In another aspect the antigen and the antibody are a single fusion protein. In another aspect the antibody comprises at least one of a light chain, a heavy chain, or a heavy and a light chain.

The present invention in another embodiment provides a vaccine composition comprising: (i) an antigen and (ii) an adjuvant, wherein the adjuvant comprises an anti-dendritic cell (DC)-specific antibody or fragment thereof conjugated to at least a portion of a TLR agonist in an amount effective to produce an immune response in a human or animal subject in need of immunostimulation. The DC-specific antibody or fragment used in the vaccine hereinabove is selected from an anti-DCIR, MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, or ASPGR. The vaccine composition further comprises antigenic peptides selected. Non-limiting examples of antigenic peptides that may be used in the vaccine composition of the present invention have been previously described in paragraph [0012]. The vaccine composition further comprises antigenic peptides selected from one or more bacterial antigens. A list of non-limiting bacterial antigens that may be used is found in paragraph [0012]. In another aspect the vaccine further comprises antigenic peptides selected from cancer peptides selected from tumor associated antigens comprising antigens previously described in paragraph [0013].

In yet another aspect the composition further comprises antigenic peptides selected from tumor associated antigens selected from CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67. In one aspect the DC-specific antibody is humanized. In another aspect the TLR agonist comprises at least one of a flagellin from Salmonella enterica, a flagellin from Vibrio cholerae, any other TLR5 agonist, a TLR7 agonist, a TLR9 agonist, or any combinations or modifications thereof. In yet another aspect the antigen is conjugated to the antibody and TLR agonist. In another aspect the antigen and the antibody are a single fusion protein. In yet another aspect the antibody comprises at least one of a light chain, a heavy chain, or a heavy and a light chain.

In yet another embodiment the instant invention discloses a method for increasing effectiveness of antigen presentation by an antigen presenting cell comprising: contacting the antigen presenting cell with a composition comprising: (i) an antigen; and (ii) an adjuvant, wherein the adjuvant comprises an anti-dendritic cell (DC)-specific antibody or binding fragment thereof conjugated to a TLR agonist, wherein the antigen and adjuvant are provided in an amount effective to produce an immune response in a human or animal subject in need of immunostimulation. Ex vivo methods of increasing effectiveness of antigen presentation by antigen presenting cells have been previously described in U.S. Patent Application Publication No. 20100135994 (Banchereau et al. 2010) and in U.S. patent application Ser. No. 13/100,684 (Banchereau et al. 2011), relevant portions of which are incorporated herein by reference.

In one aspect the DC-specific antibody or fragment is selected from an anti-DCIR, MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, or ASPGR. In a related aspect the anti-DC-specific antibody is selected from pairs of SEQ ID NOS.: 7 and 9, 11 and 13, 15 and 17, 19 and 21, 23 and 25, 27 and 29, 31 and 33, 35 and 37, 39 and 41, 43 and 45, 47 and 49, 51 and 53, 55 and 57, 59 and 61, 63 and 65, 67 and 69, 71 and 73, 75 and 77, 79 and 81, 83 and 85, 87 and 89, 91 and 93, 95 and 97, 99 and 101, 1-3 and 105, 107 and 109, 111 and 113, 115 and 117, 119 and 121, 123 and 125, 127 and 129, 131 and 132, 133 and 134.

In one aspect the composition further comprises antigenic peptides, non-limiting examples of the antigenic peptides have been described previously. In another aspect the composition further comprises antigenic peptides selected from cancer peptides as previously described. The antigenic peptides are selected from tumor associated antigens. Non-limiting examples of tumor associated antigens are described herein.

In related aspects of the method the DC-specific antibody is humanized and the composition is administered to the human or animal subject by an oral route, a nasal route, topically or as an injection (the injection is selected from intradermal, intramucosal, subcutaneous, intravenous, intraperitoneal, intramuscular, and intravenous injections). In one aspect the TLR agonist comprises at least one of a flagellin from Salmonella enterica, a flagellin from Vibrio cholerae, any other TLR5 agonist, a TLR7 agonist, a TLR9 agonist, or any combinations or modifications thereof. In another aspect the antigen is conjugated to the antibody and TLR agonist. In yet another aspect the antigen and the antibody are a single fusion protein. In another aspect the antibody comprises at least one of a light chain, a heavy chain, or a heavy and a light chain.

The present invention further provides a method for a treatment, a prophylaxis or a combination thereof against one or more cancers in a human subject comprising the steps of: (i) identifying the human subject in need of the treatment, the prophylaxis or a combination thereof against the one or more cancers and (ii) administering a vaccine composition comprising: a) an antigen and b) an adjuvant, wherein the adjuvant comprises an anti-dendritic cell (DC)-specific antibody or fragment thereof conjugated to a TLR agonist; and a pharmaceutically acceptable carrier, wherein the antigen and adjuvant are provided in an amount effective to produce an immune response for the treatment, the prophylaxis or a combination thereof against the one or more cancers.

Another embodiment of the present invention relates to a method of providing immunostimulation by activation of one or more dendritic cells (DCs) to a human subject for a prophylaxis, a therapy or a combination thereof against one or more viral, bacterial, fungal, parasitic, protozoal, and parasitic diseases, and allergic disorders comprising the steps of: i) identifying the human subject in need of immunostimulation for the prophylaxis, the therapy or a combination thereof against the one or more viral, bacterial, fungal, parasitic, protozoal, and parasitic diseases, and allergic disorders; ii) isolating one or more DCs from the human subject; iii) activating the isolated DCs with an amount of a composition effective for forming activated DCs comprising: a) an antigen and b) an adjuvant, wherein the adjuvant comprises an anti-dendritic cell (DC)-specific antibody or fragment thereof conjugated to a TLR agonist; and a pharmaceutically acceptable carrier, in an amount effective to produce an immune response in a human or animal subject in need of immunostimulation; and iv) reintroducing the activated DCs into the human subject.

In yet another embodiment the instant invention discloses an adjuvant composition comprising an anti-dendritic cell (DC)-specific antibody or fragment thereof conjugated to at least a portion of a TLR agonist wherein the anti-DC-specific antibody is selected from pairs of SEQ ID NOS.: 7 and 9, 11 and 13, 15 and 17, 19 and 21, 23 and 25, 27 and 29, 31 and 33, 35 and 37, 39 and 41, 43 and 45, 47 and 49, 51 and 53, 55 and 57, 59 and 61, 63 and 65, 67 and 69, 71 and 73, 75 and 77, 79 and 81, 83 and 85, 87 and 89, 91 and 93, 95 and 97, 99 and 101, 1-3 and 105, 107 and 109, 111 and 113, 115 and 117, 119 and 121, 123 and 125, 127 and 129, 131 and 132, 133 and 134.

In one aspect of the adjuvant composition hereinabove the composition further comprises antigenic peptides selected from human immunodeficiency virus (HIV) antigens and gene products selected from the group consisting of gag, pol, and env genes, the Nef protein, reverse transcriptase, string of HIV peptides (Hipo5), PSA (KLQCVDLHV)-tetramer, a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide (GILGFVFTL) tetramer, and Avian Flu (HA5-1), dockerin domain from C. thermocellum, measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens or combinations and modifications thereof.

In another aspect the composition further comprises antigenic peptides selected from cancer peptides selected from tumor associated antigens comprising antigens from leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia. In yet another aspect the composition further comprises antigenic peptides selected from tumor associated antigens selected from CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67. In a specific aspect the DC-specific antibody is humanized. In other related aspects the TLR agonist comprises at least one of a flagellin from Salmonella enterica, a flagellin from Vibrio cholerae, any other TLR5 agonist, a TLR7 agonist, a TLR9 agonist, or any combinations or modifications thereof and the antigen and the antibody are a single fusion protein. In another aspect the antibody comprises at least one of a light chain, a heavy chain, or a heavy and a light chain.

### BRIEF DESCRIPTION OF THE DRAWINGS

For a more complete understanding of the features and advantages of the present invention, reference is now made to the detailed description of the invention along with the accompanying figures and in which:

FIGS. 1A-1D show flagellin and the several antibody-flagellin constructs of the present invention: FIG. 1A Residues from the conserved N-terminal and C-terminal regions are together sufficient to activate TLR5—central residues are expendable for this function, FIG. 1B An example of a active configuration of flagellin fused directly to a DC-targeting antibody to direct the activation properties of the flagellin fragment specifically to cells expressing the particular DC receptor and combining immunostimulatory properties of the flagellin with those of the anti-DC receptor antibody, FIG. 1C A variant of 1B wherein fragments of the two conserved flagellin domains are separated by a linker or antigen sequence—in the example given in FIG. 3A, this configuration with a particular linker sequence is not active, FIG. 1D A variant of FIG. 1A wherein an antigen or protein-protein interaction domain (dockerin in this case) is directly linked between the flagellin and antibody domains. FIGS. 3 and 4 show examples of immune stimulation by such a variant.

FIG. 2 shows the design of the studies used to test the activity of the antibody-flagellin constructs of the present invention;

FIGS. 3A to 3C show the secretion of IL-6 when various types of cells were activated with the constructs of the present invention;

FIGS. 4A to 4D show the dose titration of the various constructs as measured by the secretion of IL-6 and IL-1 beta. The data demonstrate that the immune stimulation by the flagellin fragments becomes dependent upon DC receptor interaction (in this case LOX-1) mediated by the DC-targeting antibody portion of the construct—a control hIgG4-flagellin construct is only active at much higher concentrations. Other controls show that it is the direct fusion of the flagellin fragments to the DC-targeting antibody that accounts for the enhanced immunostimulatory potency; and

FIG. 5 shows heat maps of gene expression for the listed genes using an anti-LOX-1 antibody with or without linked flagellin. The exact combination of up-regulated immune stimulatory molecules will vary with different anti-DC receptor antibody constructs linked to the flagellin fragment because different combinations of target cells will be engaged and there will be different combinations of signaling via the flagellin and the DC-targeting antibody.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an,” and “the” are not intended to refer to only a singular entity, but include the general class of which a specific example may be used for illustration. The terminology herein is used to describe specific embodiments of the invention, but their usage does not delimit the invention, except as outlined in the claims.

As used herein the term “Antigen Presenting Cells” (APC) are cells that are capable of activating T cells, and include, but are not limited to, certain macrophages, B cells and dendritic cells. “Dendritic cells” (DCs) refers to any member of a diverse population of morphologically similar cell types found in lymphoid or non-lymphoid tissues. These cells are characterized by their distinctive morphology, high levels of surface MHC-class II expression (Steinman, et al., Ann. Rev. Immunol. 9:271 (1991); incorporated herein by reference for its description of such cells). These cells can be isolated from a number of tissue sources, and conveniently, from peripheral blood, as described herein. Dendritic cell binding proteins refers to any protein for which receptors are expressed on a dendritic cell. Examples include GM-CSF, IL-1, TNF, IL-4, CD40L, CTLA4, CD28, and FLT-3 ligand.

For the purpose of the present invention, the term “vaccine composition” is intended to mean a composition which can be administered to humans or to animals in order to induce an immune system response; this immune system response can result in a production of antibodies or simply in the activation of certain cells, in particular antigen-presenting cells, T lymphocytes and B lymphocytes. The vaccine composition can be a composition for prophylactic purposes or for therapeutic purposes, or both. As used herein, the term “antigen” refers to any antigen that can be used in a vaccine, whether it involves a whole microorganism or a portion thereof, and various types: (e.g., peptide, protein, glycoprotein, polysaccharide, glycolipid, lipopeptide, etc). They may be viral antigens, bacterial antigens, or the like; the term “antigen” also comprises the polynucleotides, the sequences of which are chosen so as to encode the antigens whose expression by the individuals to which the polynucleotides are administered is desired, in the case of the immunization technique referred to as DNA immunization. They may also be a set of antigens, in particular in the case of a multivalent vaccine composition which comprises antigens capable of protecting against several diseases, and which is then generally referred to as a vaccine combination, or in the case of a composition which comprises several different antigens in order to protect against a single disease, as is the case for certain vaccines against whooping cough or the flu, for example. The term “antibodies” refers to immunoglobulins, whether natural or partially or wholly produced artificially, e.g. recombinant. An antibody may be monoclonal or polyclonal. The antibody may, in some cases, be a member of one, or a combination immunoglobulin classes, including: IgG, IgM, IgA, IgD, and IgE.

The terms “adjuvant” or “immunoadjuvant” may be used interchangeably and refer to a substance that enhances, augments or potentiates the host's immune response to an antigen, e.g., an antigen that is part of a vaccine. Non-limiting examples of some commonly used vaccine adjuvants include insoluble aluminum compounds, calcium phosphate, liposomes, Virosomes™, ISCOMS®, microparticles (e.g., PLG), emulsions (e.g., MF59, Montanides), virus-like particles & viral vectors. Flagellin, a TLR5 agonist from Salmonella entericum is used as an adjuvant in the present invention. It will be understood that flagellin from other bacterial sources (e.g., Vibrio cholerae) may also be used, other TLR5 agonists, TLR7 agonists, TLR9 agonists, or any combinations or modifications thereof may also be used.

The term “conjugate” as used herein refers to any substance formed from the joining together of two parts. Representative conjugates in accordance with the present invention include those formed by joining together of the antigen with the antibody and the TLR agonist. The term “conjugation” refers to the process of forming the conjugate and is usually done by physical coupling, e.g. covalent binding, co-ordination covalent, or secondary binding forces, e.g. Van der Waals bonding forces. The process of linking the antigen to the antibody and the TLR agonist can also be done via a non-covalent association such as a dockerin-cohesin association (as described in U.S. Patent Publication No. 20100135994, Banchereau et al. relevant portions incorporated herein by reference) or by a direct chemical linkage by forming a peptide or chemical bond.

As used herein, the term “flagellin” refers to a flagellin protein from any source including, but not limited to, any bacterial species. The flagellin may be from a species of Salmonella (Salmonella enterica as exemplified herein) or from other species of bacteria (for e.g. Vibrio cholerae). Also specifically contemplated are fragments, variants, analogs, homologs, or derivatives of said flagellin, and combinations thereof. The various fragments, variants, analogs, homologs or derivatives described herein may be 50%, 55%, 60%, 65%, 70%, 75%, 80%, 85%, 90%, 95%, 97%, 98%, or 99% identical to a wild-type flagellin from a specific bacterial species, e.g., Salmonella.

The term “gene” is used to refer to a functional protein, polypeptide or peptide-encoding unit. As will be understood by those in the art, this functional term includes genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated

As used herein, the term “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages.

Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

As used in this application, the term “amino acid” means one of the naturally occurring amino carboxylic acids of which proteins are comprised. The term “polypeptide” as described herein refers to a polymer of amino acid residues joined by peptide bonds, whether produced naturally or synthetically. Polypeptides of less than about 10 amino acid residues are commonly referred to as “peptides.” A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

As used herein, the term “in vivo” refers to being inside the body. The term “in vitro” used as used in the present application is to be understood as indicating an operation carried out in a non-living system.

As used herein, the term “treatment” or “treating” means any administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology).

The present invention describes novel vaccine adjuvants based on targeting adjuvants with antibodies directly to antigen-presenting cells. The present invention was found to enhance vaccine efficacy by directly linking adjuvants (e.g., TLR ligands) directly to DC-targeting vaccines. As shown herein, the compositions and methods of the present invention are broadly applicable to all DC-targeting vaccines and extensible to making adjuvants with unexpected novel properties. While antigens directly linked to adjuvants (e.g., TLR's) are well known—e.g., development of Hep B vaccine linked to CpG (Dynavax) or Flu antigens linked to flagellin (Vaxigen). The present invention is an adjuvant that is directly linked to a DC-targeting vaccine (e.g., anti-DC receptor antibody fused to antigen). Several aspects of the present invention have advantages over the prior art, including dose-sparing (by sending the adjuvant directly to the antigen-presenting cell that actually receives antigen), unexpectedly, this discovery provides a method of making the agonist activity of the adjuvant strictly dependent upon the type of DC receptor targeted.

Compositions and methods described herein can be used in a prophylaxis, a therapy or a combination thereof against one or more viral, bacterial, fungal, parasitic, protozoal, and parasitic diseases, allergic disorders, and cancers. Non-limiting examples of diseases against which a prophylaxis, a therapy, alleviation of symptoms or combinations thereof can be achieved using the composition of the present invention include HIV infections, hepatitis, influenza, avian flu, herpes, genitourinary, prostate, and neurological tumors, arthritis, asthma, eczema, bacterial infections selected from anthrax, cholecystitis, bacteremia, cholangitis, urinary tract infection (UTI), listeriosis, meningococcal infections, salmonellosis, necrotizing fasciitis and streptococcal toxic shock syndrome, pneumonia, skin infections, or any combinations or modifications thereof.

Flagellin from Salmonella enterica has been exemplified in the present invention. However, a skilled artisan will understand that flagellin from many bacterial species (for e.g., Vibrio cholerae) can be substituted for the flagellin that is used herein. In addition to flagellin other TLR5 agonists may also be used. For extending the invention to other TLRs, it may be necessary to employ chemical linkages since some or all of the other known TLR ligands are non-protein. Some agonists like TLR7 and TLR9, are well described chemical entities and methods to link them to proteins, while maintaining their intrinsic TLR agonistic properties, have benne previously described. For some other agonists active compounds are known, but their protein-linking chemistries are not well described

Compositions and method described hereinabove use TLR agonists conjugated an anti-dendritic cell (DC)-specific antibody or binding fragment thereof. However, a skilled artisan will recognize that other non-TLR based ligands, agonists, or other moieties (for e.g. infammasome) may be conjugated to the antibody to achieve the desired in vivo or ex vivo effects.

It will be understood by the skilled artisan that the composition comprising the antigen-TLR agonist-antibody, may have different possible arrangements for the individual species or moieties. For example, the TLR agonist (or flagellin as used herein) may be linked to the heavy chain (ANTIBODY L CHAIN-ANTIGEN+H CHAIN-TLR AGONIST or the light-chain (ANTIBODY H CHAIN-ANTIGEN+L CHAIN-TLR AGONIST or ANTIBODY H CHAIN-ANTIGEN-TLR AGONIST+L CHAIN) of the anti-dendritic cell (DC)-specific antibody. The conjugate may also be prepared by linking the TLR agonist/flagellin through a dockerin-cohesin attachment (for e.g. ANTIBODY H CHAIN-ANTIGEN-DOCKERIN:COHESIN-FLGN). It will be understood that the flagellin used herein may be substituted or replaced by any TLR agonist that may be linked by similar chemical or physical methods. The present invention may also include the combination wherein the antigen and the antibody are a single fusion protein.

DC-targeting linked adjuvant attachment A (SEQ ID NO: 1): Vector C959 encodes rAB-
pIRES2[mAnti-DCIR_9E8_H-LV-hIgG4H-C-Flex-v1-Flgn-1-Flgn-2].
QVTLKESGPGILQPSQTLSLTCSFSGFSLSTSGMGLSWIRQPSGKGLEWLAHIYWDDDKRY
NPSLKSRLTISKDTSSNQVFLKITIVDTADAATYYCARSSHYYGYGYGGYFDVWGAGTTV
TVSSAKTKGPSVFPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGPPCPPCPAPEFEGGPS
VFLFPPKPKDTLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYSRLTVDKSRWQE
GNVFSCSVMHEALHNHYTQKSLSLSLGK [symbol] QTPTNTISVTPTNNSTPTNNSNPKPNP [symbol] IERL
SSGLRINSAKDDAAGQAIANRFTANIKGLTQASRNANDGISIAQTTEGALNEINNNLQR
VRELAVQSANSTNSQSDLDSIQAEITQRLNEIDRVSGQTQFNGVKVLAQDNTLTIQVG
ANDGETIDIDLKQINSQTLGLDSLNVQ [symbol] QPELAEAAAKTTENPLQKIDAALAQVDALRS
DLGAVQNRFNSAITNLGNTVNNLSEARSRIEDSDYATEVSNMSRAQILQAS

Bold is the N-terminal part of phase-2 flagellin [Salmonella enterica] gb|AAL30512.1|AF425736_{—}1 residues 14-161.

Underlined is the C-terminal part of phase-2 flagellin [Salmonella enterica] gb|AAL30512.1|AF425736_{—}1 residues 405-484.

Italics is a flexible linker sequence from gb|AAT79550.1| cellulosomal anchoring scaffoldin B precursor [Bacteroides cellulosolvens].

The amino terminus up to the first AS sequence is the heavy chain of mouse Anti-DCIR_{—}9E8 variable region fused to −hIgG4H constant region. AS sequences in bold-italics are joining sequences from construction of the expression vector.

When co-transfected with appropriate L chain expression vector into mammalian cells (e.g., CHO—S cells) this vector directs efficient secretion of a typical embodiment of a DC-targeting agent linked to a flagellin-based adjuvant.

C1099 encodes mouse Anti-DCIR_9E8_H-LV-hIgG4H-C-Dockerin-v2-Flgn-1-Flgn-2].
(SEQ ID NO: 2)
QVTLKESGPGILQPSQTLSLTCSFSGFSLSTSGMGLSWIRQPSGKGLEWLAHIYWDDDKRY
NPSLKSRLTISKDTSSNQVFLKITIVDTADAATYYCARSSHYYGYGYGGYFDVWGAGTTV
TVSSAKTKGPSVFPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGPPCPPCPAPEFEGGPS
VFLFPPKPKDTLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYSRLTVDKSRWQE
GNVFSCSVMHEALHNHYTQKSLSLSLGK [symbol] SEDTQPPAPTLIGDVNADGKIDSTDLTLLKRYL
LRSATLTEEKILNADTDGNGTVNSTDLNYLKKYILRVISVFPAEGNKPPTPTPTKTPVATPSPTQPL
FTPSFKDVT [symbol] IERLSSGLRINSAKDDAAGQAIANRFTANIKGLTQASRNANDGISIAQTT
EGALNEINNNLQRVRELAVQSANSTNSQSDLDSIQAEITQRLNEIDRVSGQTQFNGVK
VLAQDNTLTIQVGANDGETIDIDLKQINSQTLGLDSLNVQ [symbol] QPELAEAAAKTTENPLQ
KIDAALAQVDALRSDLGAVQNRFNSAITNLGNTVNNLSEARSRIEDSDYATEVSNMSRAQI
LQAS

Italics are |YP_{—}001036450.1| alpha-L-arabinofuranosidase B [Clostridium thermocellum ATCC 27405| residues 166-274—this is a dockerin domain that functions fully for binding (e.g., cohesin-antigen fusions) when fused between other domains.

Bold is the N-terminal part of phase-2 flagellin [Salmonella enterica] gb|AAL30512.1|AF425736_{—}1 residues 14-161.

Underlined is the C-terminal part of phase-2 flagellin [Salmonella enterica] gb|AAL30512.1|AF425736_{—}1 residues 405-484.

AS sequences in bold-italics are joining sequences from construction of the expression vector.

When co-transfected with appropriate L chain expression vector into mammalian cells (e.g., CHO—S cells) this vector directs efficient secretion of a typical embodiment of a DC-targeting agent linked to a flagellin-based adjuvant and linked to cohesin-antigen via the dockerin domain. In related embodiments—any desired antigen can also be directly fused in place of the dockerin domain.

C566 E. coli-pET28 vector encoding expression of
[Cohesin-var1-FluM1-6xHis]

(SEQ ID NO: 3)

MDLDAVRIKVDTVNAKPGDTVNIPVRFSGIPSKGIANADFVYSYDPNV
LEIIEIKPGELIVDPNPTKSFDTAVYPDRKMIVFLFAEDSGTGAYAIT
KDGVFATIVAKVKEGAPNGLSVIKFVEVGGFANNDLVEQKTQFFD
GGVNVGDTTEPATPTTPVTTPTTTDDLDAASLLTEVETYVLSIIP
SGPLKAEIAQRLEDVFAGKNTDLEVLMEWLKTRPILSPLTKGIL
GFVFTLTVPSERGLQRRRFVQNALNGNGDPNNMDKAVKLYR
KLKREITFHGAKEIALSYSAGALASCMGLIYNRMGAVTTEVAFG
LVCATCEQIADSQHRSHRQMVTTTNPLIRHENRMVLASTTAKA
MEQMAGSSEQAAEAMDIASQARQMVQAMRTIGTHPSSSAGL
KDDLLENLQAYQKRMGVQMQRFKLEHHHHHH

Bold is the cohesin domain showing an underlined single C to A change that maintains dockerin binding and 3 C residues (bold-underlined) that permit site-specific maleimide linkage of TLRL adducts. Underlined is the Flu M1 antigen sequence. AS sequences in bold-italics are joining sequences from construction of the expression vector.

In related forms—any cohesin-antigen with free cys residues can be conveniently decorated with TLR7-L compound and linked with any anti-DC receptor-dockerin-antigen vaccine.

C1450 encodes mouse Anti-DCIR_9E8_H-LV-hIgG4H-C-
Flex-v1-v1C2 (SEQ ID NO: 4):
QVTLKESGPGILQPSQTLSLTCSFSGFSLSTSGMGLSWIRQPSGKGL
EWLAHIYWDDDKRYNPSLKSRLTISKDTSSNQVFLKITIVDTADAATYY
CARSSHYYGYGYGGYFDVWGAGTTVTVSSAKTKGPSVFPLAPCSRS
TSESTAALGCLVKDYFPEPVTVSWNSGALTSGVHTFPAVLQSSGLYS
LSSVVTVPSSSLGTKTYTCNVDHKPSNTKVDKRVESKYGPPCPPCPA
PEFEGGPSVFLFPPKPKDTLMISRTPEVTCVVVDVSQEDPEVQFNWY
VDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYP
SDIAVEWESNGQPENNYKTTPPVLDSDGSFFLYSRLTVDKSRWQEGNV
FSCSVMHEALHNHYTQKSLSLSLGK [symbol] QTPTNTISVTPTNNSTPTN
NSNPKPNP [symbol] CQTPTNTISVTPTNNSTPTNNSNPKPCP

[symbol]

Bold is a flexible linker sequence bearing two C residues (underlined) for site-specific linking TLRL adducts. When co-transfected with appropriate L chain expression vector into mammalian cells (e.g., CHO—S cells) this vector directs efficient secretion of a typical embodiment of a DC-targeting agent linked to a chemical-based adjuvant. In related embodiments—other vectors can be prepared with any desired antigen directly fused to the C-terminal codon. AS sequences in bold-italics are joining sequences from construction of the expression vector. Italics is a flexible linker (supra).

C1180 directs the expression of a mammalian cell
expressed 6xHis-Cohesin-Flgn-1-Flgn-2 fusion
protein (SEQ ID NO: 5):
LDITSHHHHHHDDLDAVRIKVDTVNAKPGDTVRIPVRFSGIPSKGIAN
CDFVYSYDPNVLEIIEIEPGDIIVDPNPDKSFDTAVYPDRKIIVFLFAED
SGTGAYAITKDGVFATIVAKVKEGAPNGLSVIKFVEVGGFANNDLVE
QKTQFFDGGVNVGDTTEPATPTTPVTTPTTTDDLDA [symbol] IERLS
SGLRINSAKDDAAGQAIANRFTANIKGLTQASRNANDGISIAQTTEGALN
EINNNLQRVRELAVQSANSTNSQSDLDSIQAEITQRLNEIDRVSGQTQF
NGVKVLAQDNTLTIQVGANDGETIDIDLKQINSQTLGLDSLNVQASQPE
LAEAAAKTTENPLQKIDAALAQVDALRSDLGAVQNRFNSAITNLGNTVN
NLSEARSRIEDSDYATEVSNMSRAQILQAS

Italics is the cohesin domain. Bold is the N-terminal part of phase-2 flagellin [Salmonella enterica] gb|AAL30512.1|AF425736_{—}1 residues 14-161. Underlined is the C-terminal part of phase-2 flagellin [Salmonella enterica] gb|AAL30512.1|AF425736_{—}1 residues 405-484. AS sequences in bold-italics are joining sequences from construction of the expression vector. This form permits linking of functional Flgn to any anti-DC receptor-Dockerin-antigen vaccine.

Some other non-limiting examples of constructs with different DC-specific antibodies or fragments are presented herein below:

Anti_CLEC_6_9B9.2G12_Hv-V-hIgG4H-C (SEQ ID NO: 6):
ATGGGCAGGCTTACTTCTTCATTCTTGCTACTGATTGTCCCTGCATATGTCCTGTCCCA
GGTTACTCTGAAAGAGTCTGGCCCTGGGATATTGCAGCCCTCCCAGACCCTCAGTCTG
ACCTGTTCTTTCTCTGGGTTTTCACTGAGCACTTCTGGTATGAGTGTAGGCTGGATTCG
TCAGCCTTCAGGGAAGGGTCTGGAGTGGCTGGCTCACATTTGGTGGAATGATGATAAG
TACTATAATCCAGTCCTGAAAAGCCGGCTCACAATCTCCAAGGAGACCTCCAACAACC
AGGTATTCCTCAAGATCGCCAGTGTGGTCTCTGCAGATACTGCCACATACTACTGTGCT
CGATTCTATGGTAACTGTCTTGACTACTGGGGCCAAGGCACCACTCTCACAGTCTCCTC
GGCCAAAACAaagggcccATCCGTCTTCCCCCTGGCGCCCTGCTCCAGGAGCACCTCCGAG
AGCACAGCCGCCCTGGGCTGCCTGGTCAAGGACTACTTCCCCGAACCGGTGACGGTGT
CGTGGAACTCAGGCGCCCTGACCAGCGGCGTGCACACCTTCCCGGCTGTCCTACAGTC
CTCAGGACTCTACTCCCTCAGCAGCGTGGTGACCGTGCCCTCCAGCAGCTTGGGCACG
AAGACCTACACCTGCAACGTAGATCACAAGCCCAGCAACACCAAGGTGGACAAGAGA
GTTGAGTCCAAATATGGTCCCCCATGCCCACCCTGCCCAGCACCTGAGTTCGAAGGGG
GACCATCAGTCTTCCTGTTCCCCCCAAAACCCAAGGACACTCTCATGATCTCCCGGACC
CCTGAGGTCACGTGCGTGGTGGTGGACGTGAGCCAGGAAGACCCCGAGGTCCAGTTC
AACTGGTACGTGGATGGCGTGGAGGTGCATAATGCCAAGACAAAGCCGCGGGAGGAG
CAGTTCAACAGCACGTACCGTGTGGTCAGCGTCCTCACCGTCCTGCACCAGGACTGGC
TGAACGGCAAGGAGTACAAGTGCAAGGTCTCCAACAAAGGCCTCCCGTCCTCCATCGA
GAAAACCATCTCCAAAGCCAAAGGGCAGCCCCGAGAGCCACAGGTGTACACCCTGCC
CCCATCCCAGGAGGAGATGACCAAGAACCAGGTCAGCCTGACCTGCCTGGTCAAAGG
CTTCTACCCCAGCGACATCGCCGTGGAGTGGGAGAGCAATGGGCAGCCGGAGAACAA
CTACAAGACCACGCCTCCCGTGCTGGACTCCGACGGCTCCTTCTTCCTCTACAGCAGGC
TAACCGTGGACAAGAGCAGGTGGCAGGAGGGGAATGTCTTCTCATGCTCCGTGATGCA
TGAGGCTCTGCACAACCACTACACACAGAAGAGCCTCTCCCTGTCTCTGGGTAAAGCT
AGCTGATTAATTAA
Anti_CLEC_6_9B9.2G12_Hv-V-hIgG4H-C (SEQ ID NO: 7):
MGRLTSSFLLLIVPAYVLSQVTLKESGPGILQPSQTLSLTCSFSGFSLSTSGMSVGWIRQPSG
KGLEWLAHIWWNDDKYYNPVLKSRLTISKETSNNQVFLKIASVVSADTATYYCARFYGN
CLDYWGQGTTLTVSSAKTKGPSVFPLAPCSRSTSESTAALGCLVKDYFPEPVTVSWNSGA
LTSGVHTFPAVLQSSGLYSLSSVVTVPSSSLGTKTYTCNVDHKPSNTKVDKRVESKYGPPC
PPCPAPEFEGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPREPQ
VYTLPPSQEEMTKNQVSLTCLVKGFYPSDIAVEWESNGQPENNYKTTPPVLDSDGSFFLYS
RLTVDKSRWQEGNVFSCSVMHEALHNHYTQKSLSLSLGKAS
Anti_CLEC_6_9B9.2G12_Kv-V-hIgGK-C (SEQ ID NO: 8):
ATGATGTCCTCTGCTCAGTTCCTTGGTCTCCTGTTGCTCTGTTTTCAAGGTACCAGATGT
GATATCCAGATGACACAGACTACATCCTCCCTGTCTGCCTCTCTGGGAGACAGAGTCA
CCATCAGTTGCAGGGCAAGTCAGGACATTAGCAATTATTTAAACTGGTATCAGCAGAA
ACCAGATGGAACTGTTAAACTCCTGATCTACTACACATCAATATTACAATTAGGAGTC
CCATCAAGATTCAGTGGCAGTGGGTCTGAAACAGATTATTCTCTCACCATTAGCAACC
TGGAGCAAGAAGATATTGCCACTTACTTTTGCCAACAGGGTGATTCGCTTCCATTCAC
GTTCGGCTCGGGGACAAAGCTCGAGATCAAACGAACTGTGGCTGCACCATCTGTCTTC
ATCTTCCCGCCATCTGATGAGCAGTTGAAATCTGGAACTGCCTCTGTTGTGTGCCTGCT
GAATAACTTCTATCCCAGAGAGGCCAAAGTACAGTGGAAGGTGGATAACGCCCTCCA
ATCGGGTAACTCCCAGGAGAGTGTCACAGAGCAGGACAGCAAGGACAGCACCTACAG
CCTCAGCAGCACCCTGACGCTGAGCAAAGCAGACTACGAGAAACACAAAGTCTATGC
CTGCGAAGTCACCCATCAGGGCCTGAGCTCGCCCGTCACAAAGAGCTTCAACAGGGG
AGAGTGTTAG
Anti_CLEC_6_9B9.2G12_Kv-V-hIgGK-C (SEQ ID NO: 9):
MMSSAQFLGLLLLCFQGTRCDIQMTQTTSSLSASLGDRVTISCRASQDISNYLNWYQQKPD
GTVKLLIYYTSILQLGVPSRFSGSGSETDYSLTISNLEQEDIATYFCQQGDSLPFTFGSGTKL
EIKRTVAAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
Anti-ASGPR_49C11_7H-LV-hIgG4H-C (SEQ ID NO: 10):
ATGAGAGCGCTGATTCTTTTGTGCCTGTTCACAGCCTTTCCTGGTATCCTGTCTGATGT
GCAGCTTCAGGAGTCAGGACCTGACCTGGTGAAACCTTCTCAGTCACTTTCACTCACCT
GCACTGTCACTGGCTACTCCATCACCAGTGGTTATAGCTGGCACTGGATCCGGCAGTTT
CCAGGAAACAAACTGGAATGGATGGGCTACATACTCTTCAGTGGTAGCACTAACTACA
ACCCATCTCTGAAAAGTCGAATCTCTATCACTCGAGACACATCCAAGAACCAGTTCTT
CCTGCAGTTGAATTCTGTGACTACTGAGGACACAGCCACATATTTCTGTGCAAGATCT
AACTATGGTTCCTTTGCTTCCTGGGGCCAAGGGACTCTGGTCACTGTCTCTGCAGCCAA
AACAACGGGCCCATCCGTCTTCCCCCTGGCGCCCTGCTCCAGGAGCACCTCCGAGAGC
ACAGCCGCCCTGGGCTGCCTGGTCAAGGACTACTTCCCCGAACCGGTGACGGTGTCGT
GGAACTCAGGCGCCCTGACCAGCGGCGTGCACACCTTCCCGGCTGTCCTACAGTCCTC
AGGACTCTACTCCCTCAGCAGCGTGGTGACCGTGCCCTCCAGCAGCTTGGGCACGAAG
ACCTACACCTGCAACGTAGATCACAAGCCCAGCAACACCAAGGTGGACAAGAGAGTT
GAGTCCAAATATGGTCCCCCATGCCCACCCTGCCCAGCACCTGAGTTCGAAGGGGGAC
CATCAGTCTTCCTGTTCCCCCCAAAACCCAAGGACACTCTCATGATCTCCCGGACCCCT
GAGGTCACGTGCGTGGTGGTGGACGTGAGCCAGGAAGACCCCGAGGTCCAGTTCAAC
TGGTACGTGGATGGCGTGGAGGTGCATAATGCCAAGACAAAGCCGCGGGAGGAGCAG
TTCAACAGCACGTACCGTGTGGTCAGCGTCCTCACCGTCCTGCACCAGGACTGGCTGA
ACGGCAAGGAGTACAAGTGCAAGGTCTCCAACAAAGGCCTCCCGTCCTCCATCGAGA
AAACCATCTCCAAAGCCAAAGGGCAGCCCCGAGAGCCACAGGTGTACACCCTGCCCC
CATCCCAGGAGGAGATGACCAAGAACCAGGTCAGCCTGACCTGCCTGGTCAAAGGCT
TCTACCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAGGCT
AACCGTGGACAAGAGCAGGTGGCAGGAGGGGAATGTCTTCTCATGCTCCGTGATGCAT
GAGGCTCTGCACAACCACTACACACAGAAGAGCCTCTCCCTGTCTCTGGGTAAAGCTA
GCTGATTAATTAA
Anti-ASGPR_49C11_7H-LV-hIgG4H-C (SEQ ID NO: 11):
MRALILLCLFTAFPGILSDVQLQESGPDLVKPSQSLSLTCTVTGYSITSGYSWHWIRQFPGN
KLEWMGYILFSGSTNYNPSLKSRISITRDTSKNQFFLQLNSVTTEDTATYFCARSNYGSFAS
WGQGTLVTVSAAKTTGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRVESKYGPPCPPCP
APEFEGGPSVFLFPPKPKDTLMISRTPEVTCVVVDVSQEDPEVQFNWYVDGVEVHNAKTK
PREEQFNSTYRVVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSDGSFFLYSRLTV
DKSRWQEGNVFSCSVMHEALHNHYTQKSLSLSLGKAS
Anti-ASGPR_49C11_7K-LV-hIgGK-C (SEQ ID NO: 12):
ATGGATTTTCAAGTGCAGATTTTCAGCTTCCTGCTAATCAGTGCCTCAGTCATAATATC
CAGAGGACAAATTGTTCTCACCCAGTCTCCAGCAATCATGTCTGCATCTCCAGGGGAG
AAGGTCACCATGACCTGCAGTGCCAGCTCAAGTGTAAGTCACATGCACTGGTACCAGC
AGAAGTCAGGCACTTCCCCCAAAAGATGGATTTATGACACATCCAGACTGGCTTCTGG
AGTCCCTGCTCGCTTCAGTGGCAGTGGGTCTGGGACCTCTTACTCTCTCACAATCAGCA
GCATGGAGGCTGAAGATGCTGCCACTTATTACTGCCAGCAGTGGAGTAGTCACCCATG
GTCGTTCGGTGGAGGCACCAAACTCGAGATCAAACGAACTGTGGCTGCACCATCTGTC
TTCATCTTCCCGCCATCTGATGAGCAGTTGAAATCTGGAACTGCCTCTGTTGTGTGCCT
GCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTAC
AGCCTCAGCAGCACCCTGACGCTGAGCAAAGCAGACTACGAGAAACACAAAGTCTAT
GCCTGCGAAGTCACCCATCAGGGCCTGAGCTCGCCCGTCACAAAGAGCTTCAACAGGG
GAGAGTGTTAG
Anti-ASGPR_49C11_7K-LV-hIgGK-C (SEQ ID NO: 13):
MDFQVQIFSFLLISASVIISRGQIVLTQSPAIMSASPGEKVTMTCSASSSVSHMHWYQQKSG
TSPKRWIYDTSRLASGVPARFSGSGSGTSYSLTISSMEAEDAATYYCQQWSSHPWSFGGGT
KLEIKRTVAAPSVFIFPPSDEQLKSGTASVVCLLNNFYPREAKVQWKVDNALQSGNSQESV
TEQDSKDSTYSLSSTLTLSKADYEKHKVYACEVTHQGLSSPVTKSFNRGEC
Anti-ASGPR_4G2.2_Hv-V-hIgG4H-C (SEQ ID NO: 14):
ATGGCTTGGGTGTGGACCTTGCTATTCCTGATGGCAGCTGCCCAAAGTGCCCAAGCAC
AGATCCAGTTGGTGCAGTCTGGACCTGAGCTGAAGAAGCCTGGAGAGACAGTCAAGA
TCTCCTGCAAGGCTTCTGGGTATACCTTCACAAACTATGGAATGAACTGGGTGAAGCA
GGTTCCAGGAAAAGGTTTAAGGTGGATGGGCTGGATGGACACCTTCACTGGAGAGCC
AACATATGCTGATGACTTCAAGGGACGGTTTGCCTTCTCTTTGGAAACCTCTGCCAGCA
CTGCCTATTTGCAGATCAACAGCCTCAAAAATGAGGACACGGCTACTTATTTCTGTGC
AAGAGGGGGGATTTTACGACTCAACTACTTTGACTACTGGGGCCAAGGCACCACTCTC
ACAGTCTCCTCAGCCAAAACGAAGGGCCCATCCGTCTTCCCCCTGGCGCCCTGCTCCA
GGAGCACCTCCGAGAGCACAGCCGCCCTGGGCTGCCTGGTCAAGGACTACTTCCCCGA
ACCGGTGACGGTGTCGTGGAACTCAGGCGCCCTGACCAGCGGCGTGCACACCTTCCCG
GCTGTCCTACAGTCCTCAGGACTCTACTCCCTCAGCAGCGTGGTGACCGTGCCCTCCAG
CAGCTTGGGCACGAAGACCTACACCTGCAACGTAGATCACAAGCCCAGCAACACCAA
GGTGGACAAGAGAGTTGAGTCCAAATATGGTCCCCCATGCCCACCCTGCCCAGCACCT
GAGTTCGAAGGGGGACCATCAGTCTTCCTGTTCCCCCCAAAACCCAAGGACACTCTCA
TGATCTCCCGGACCCCTGAGGTCACGTGCGTGGTGGTGGACGTGAGCCAGGAAGACCC
CGAGGTCCAGTTCAACTGGTACGTGGATGGCGTGGAGGTGCATAATGCCAAGACAAA
GCCGCGGGAGGAGCAGTTCAACAGCACGTACCGTGTGGTCAGCGTCCTCACCGTCCTG
CACCAGGACTGGCTGAACGGCAAGGAGTACAAGTGCAAGGTCTCCAACAAAGGCCTC
CCGTCCTCCATCGAGAAAACCATCTCCAAAGCCAAAGGGCAGCCCCGAGAGCCACAG
GTGTACACCCTGCCCCCATCCCAGGAGGAGATGACCAAGAACCAGGTCAGCCTGACCT
GCCTGGTCAAAGGCTTCTACCCCAGCGACATCGCCGTGGAGTGGGAGAGCAATGGGC
AGCCGGAGAACAACTACAAGACCACGCCTCCCGTGCTGGACTCCGACGGCTCCTTCTT
CCTCTACAGCAGGCTAACCGTGGACAAGAGCAGGTGGCAGGAGGGGAATGTCTTCTC
ATGCTCCGTGATGCATGAGGCTCTGCACAACCACTACACACAGAAGAGCCTCTCCCTG
TCTCTGGGTAAAGCTAGCTGATTAATTAA
Anti-ASGPR_4G2.2_Hv-V-hIgG4H-C (SEQ ID NO: 15):
MAWVWTLLFLMAAAQSAQAQIQLVQSGPELKKPGETVKISCKASGYTFTNYGMNWVKQ
VPGKGLRWMGWMDTFTGEPTYADDFKGRFAFSLETSASTAYLQINSLKNEDTATYFCAR
GGILRLNYFDYWGQGTTLTVSSAKTKGPSVFPLAPCSRSTSESTAALGCLVKDYFPEPVTV
SWNSGALTSGVHTFPAVLQSSGLYSLSSVVTVPSSSLGTKTYTCNVDHKPSNTKVDKRVES
KYGPPCPPCPAPEFEGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGKAS
Anti-ASGPR_4G2.2_Kv-V-hIgGK-C (SEQ ID NO: 16):
ATGAAGTTTCCTTCTCAACTTCTGCTCTTACTGCTGTTTGGAATCCCAGGCATGATATG
TGACATCCAGATGACACAATCTTCATCCTCCTTTTCTGTATCTCTAGGAGACAGAGTCA
CCATTACTTGCAAGGCAAGTGAGGACATATATAATCGGTTAGGCTGGTATCAGCAGAA
ACCAGGAAATGCTCCTAGGCTCTTAATATCTGGTGCAACCAGTTTGGAAACTGGGGTT
CCTTCAAGATTCAGTGGCAGTGGATCTGGAAAGGATTACGCTCTCAGCATTACCAGTC
TTCAGACTGAAGATCTTGCTACTTATTACTGTCAACAGTGTTGGACTTCTCCGTACACG
TTCGGAGGGGGGACCAAGCTCGAGATCAAACGAACTGTGGCTGCACCATCTGTCTTCA
TCTTCCCGCCATCTGATGAGCAGTTGAAATCTGGAACTGCCTCTGTTGTGTGCCTGCTG
AATAACTTCTATCCCAGAGAGGCCAAAGTACAGTGGAAGGTGGATAACGCCCTCCAAT
CGGGTAACTCCCAGGAGAGTGTCACAGAGCAGGACAGCAAGGACAGCACCTACAGCC
TCAGCAGCACCCTGACGCTGAGCAAAGCAGACTACGAGAAACACAAAGTCTATGCCT
GCGAAGTCACCCATCAGGGCCTGAGCTCGCCCGTCACAAAGAGCTTCAACAGGGGAG
AGTGTTAG
Anti-ASGPR_4G2.2_Kv-V-hIgGK-C (SEQ ID NO: 17):
MKFPSQLLLLLLFGIPGMICDIQMTQSSSSFSVSLGDRVTITCKASEDIYNRLGWYQQKPGN
APRLLISGATSLETGVPSRFSGSGSGKDYALSITSLQTEDLATYYCQQCWTSPYTFGGGTKL
EIKRTVAAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
Anti-ASGPR_5F10H-LV-hIgG4H-C (SEQ ID NO: 18):
ATGGGATGGAGCTGGATCTTTCTCTTTCTCTTGTCAGGAACTGGAGGTGTCCTCTCTGA
GGTCCAGCTGCAACAGTCTGGACCTGAGCTGGTGAAGCCTGGGGCTTCAGTGAAGATG
TCCTGCAAGGCTTCTGGATACACCTTCACTGACTACTACATGAAGTGGGTGAAGCAGA
GCCATGGAAAGAGCCTTGAGTGGATTGGAGATATTAATCCTAACTATGGTGATACTTT
CTACAACCAGAAGTTCGAGGGCAAGGCCACATTGACTGTAGACAAATCCTCCAGGAC
AGCCTACATGCAGCTCAACAGCCTGACATCTGAGGACTCTGCAGTCTATTATTGTGGA
AGAGGGGACTATGGATACTTCGATGTCTGGGGCGCAGGGACCACGGTCACCGTCTCCT
CAGCCAAAACAAAGGGCCCATCCGTCTTCCCCCTGGCGCCCTGCTCCAGGAGCACCTC
CGAGAGCACAGCCGCCCTGGGCTGCCTGGTCAAGGACTACTTCCCCGAACCGGTGACG
GTGTCGTGGAACTCAGGCGCCCTGACCAGCGGCGTGCACACCTTCCCGGCTGTCCTAC
AGTCCTCAGGACTCTACTCCCTCAGCAGCGTGGTGACCGTGCCCTCCAGCAGCTTGGG
CACGAAGACCTACACCTGCAACGTAGATCACAAGCCCAGCAACACCAAGGTGGACAA
GAGAGTTGAGTCCAAATATGGTCCCCCATGCCCACCCTGCCCAGCACCTGAGTTCGAA
GGGGGACCATCAGTCTTCCTGTTCCCCCCAAAACCCAAGGACACTCTCATGATCTCCC
GGACCCCTGAGGTCACGTGCGTGGTGGTGGACGTGAGCCAGGAAGACCCCGAGGTCC
AGTTCAACTGGTACGTGGATGGCGTGGAGGTGCATAATGCCAAGACAAAGCCGCGGG
AGGAGCAGTTCAACAGCACGTACCGTGTGGTCAGCGTCCTCACCGTCCTGCACCAGGA
CTGGCTGAACGGCAAGGAGTACAAGTGCAAGGTCTCCAACAAAGGCCTCCCGTCCTCC
ATCGAGAAAACCATCTCCAAAGCCAAAGGGCAGCCCCGAGAGCCACAGGTGTACACC
CTGCCCCCATCCCAGGAGGAGATGACCAAGAACCAGGTCAGCCTGACCTGCCTGGTCA
AAGGCTTCTACCCCAGCGACATCGCCGTGGAGTGGGAGAGCAATGGGCAGCCGGAGA
ACAACTACAAGACCACGCCTCCCGTGCTGGACTCCGACGGCTCCTTCTTCCTCTACAGC
AGGCTAACCGTGGACAAGAGCAGGTGGCAGGAGGGGAATGTCTTCTCATGCTCCGTG
ATGCATGAGGCTCTGCACAACCACTACACACAGAAGAGCCTCTCCCTGTCTCTGGGTA
AAGCTAGCTGA
Anti-ASGPR_5F10H-LV-hIgG4H-C (SEQ ID NO: 19):
MGWSWIFLFLLSGTGGVLSEVQLQQSGPELVKPGASVKMSCKASGYTFTDYYMKWVKQ
SHGKSLEWIGDINPNYGDTFYNQKFEGKATLTVDKSSRTAYMQLNSLTSEDSAVYYCGRG
DYGYFDVWGAGTTVTVSSAKTKGPSVFPLAPCSRSTSESTAALGCLVKDYFPEPVTVSWN
SGALTSGVHTFPAVLQSSGLYSLSSVVTVPSSSLGTKTYTCNVDHKPSNTKVDKRVESKYG
PPCPPCPAPEFEGGPSVFLFPPKPKDTLMISRTPEVTCVVVDVSQEDPEVQFNWYVDGVEV
HNAKTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSDGSFF
LYSRLTVDKSRWQEGNVFSCSVMHEALHNHYTQKSLLSLGKAS
Anti-ASGPR_5F10K-LV-hIgGK-C (SEQ ID NO: 20):
ATGGAGACACATTCTCAGGTCTTTGTATACATGTTGCTGTGGTTGTCTGGTGTTGAAGG
AGACATTGTGATGACCCAGTCTCACAAATTCATGTCCACATCAGTAGGAGACAGGGTC
AGCATCACCTGCAAGGCCAGTCAGGATGTGGGTACTGCTGTAGCCTGGTATCAACAGA
AACCAGGGCAATCTCCTAAACTACTGATTTACTGGGCATCCACCCGGCACACTGGAGT
CCCTGATCGCTTCACAGGCAGTGGATCTGGGACAGATTTCACTCTCACCATTAACAAT
GTGCAGTCTGAAGACTTGGCAGATTATTTCTGTCAGCAATATAGCAGCAATCCGTACA
TGTTCGGAGGGGGGACCAAGCTCGAGATCAAACGAACTGTGGCTGCACCATCTGTCTT
CATCTTCCCGCCATCTGATGAGCAGTTGAAATCTGGAACTGCCTCTGTTGTGTGCCTGC
TGAATAACTTCTATCCCAGAGAGGCCAAAGTACAGTGGAAGGTGGATAACGCCCTCCA
ATCGGGTAACTCCCAGGAGAGTGTCACAGAGCAGGACAGCAAGGACAGCACCTACAG
CCTCAGCAGCACCCTGACGCTGAGCAAAGCAGACTACGAGAAACACAAAGTCTATGC
CTGCGAAGTCACCCATCAGGGCCTGAGCTCGCCCGTCACAAAGAGCTTCAACAGGGG
AGAGTGTTAG
Anti-ASGPR_5F10K-LV-hIgGK-C (SEQ ID NO: 21):
METHSQVFVYMLLWLSGVEGDIVMTQSHKFMSTSVGDRVSITCKASQDVGTAVAWYQQ
KPGQSPKLLIYWASTRHTGVPDRFTGSGSGTDFTLTINNVQSEDLADYFCQQYSSNPYMFG
GGTKLEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC
Anti-ASGPR1H11_H-V-hIgG4H-C (SEQ ID NO: 22):
ATGGGATGGAGCTGGATCTTTCTCTTTCTCCTGTCAGGAACTGCAGGTGTCCTCTCTGA
GGTCCAGCTGCAACAGTCTGGACCTGAGTTGGTGAAGCCTGGGGCTTCAGTGAAGATA
TCCTGCAAGACTTCTGGATACACATTCACTGAATACACCATGCACTGGGTGAGGCAGA
GCCATGGAAAGAGCCTTGAGTGGATTGGAGGTATTAATCCTATCAATGGTGGTCCTAC
CTACAACCAGAAGTTCAAGGGCAAGGCCACATTGACTGTTGACAAGTCCTCCAGCACA
GCCTACATGGAGCTCCGCAGCCTGACATCTGAGGACTCTGCAGTCTATTACTGTGCAA
GATGGGACTATGGTAGTCGAGATGTTATGGACTACTGGGGTCAAGGAACCTCAGTCAC
CGTCTCCTCAGCCAAAACGAAGGGCCCATCCGTCTTCCCCCTGGCGCCCTGCTCCAGG
AGCACCTCCGAGAGCACAGCCGCCCTGGGCTGCCTGGTCAAGGACTACTTCCCCGAAC
CGGTACCGGTGACGGTGTCGTGGAACTCAGGCGCCCTGACCAGCGGCGTGCACACCTT
CCCGGCTGTCCTACAGTCCTCAGGACTCTACTCCCTCAGCAGCGTGGTGACCGTGCCCT
CCAGCAGCTTGGGCACGAAGACCTACACCTGCAACGTAGATCACAAGCCCAGCAACA
CCAAGGTGGACAAGAGAGTTGAGTCCAAATATGGTCCCCCATGCCCACCCTGCCCAGC
ACCTGAGTTCGAAGGGGGACCATCAGTCTTCCTGTTCCCCCCAAAACCCAAGGACACT
CTCATGATCTCCCGGACCCCTGAGGTCACGTGCGTGGTGGTGGACGTGAGCCAGGAAG
ACCCCGAGGTCCAGTTCAACTGGTACGTGGATGGCGTGGAGGTGCATAATGCCAAGAC
AAAGCCGCGGGAGGAGCAGTTCAACAGCACGTACCGTGTGGTCAGCGTCCTCACCGTC
CTGCACCAGGACTGGCTGAACGGCAAGGAGTACAAGTGCAAGGTCTCCAACAAAGGC
CTCCCGTCCTCCATCGAGAAAACCATCTCCAAAGCCAAAGGGCAGCCCCGAGAGCCAC
AGGTGTACACCCTGCCCCCATCCCAGGAGGAGATGACCAAGAACCAGGTCAGCCTGA
CCTGCCTGGTCAAAGGCTTCTACCCCAGCGACATCGCCGTGGAGTGGGAGAGCAATGG
GCAGCCGGAGAACAACTACAAGACCACGCCTCCCGTGCTGGACTCCGACGGCTCCTTC
TTCCTCTACAGCAGGCTAACCGTGGACAAGAGCAGGTGGCAGGAGGGGAATGTCTTCT
CATGCTCCGTGATGCATGAGGCTCTGCACAACCACTACACACAGAAGAGCCTCTCCCT
GTCTCTGGGTAAAGCTAGCTGA
Anti-ASGPR1H11_H-V-hIgG4H-C (SEQ ID NO: 23):
MGWSWIFLFLLSGTAGVLSEVQLQQSGPELVKPGASVKISCKTSGYTFTEYTMHWVRSHG
KSLEWIGGINPINGGPTYNQKFKGKATLTVDKSSSTAYMELRSLTSEDSAVYYCARWDYG
SRDVMDYWGQGTSVTVSSAKTKGPSVFPLAPCSRSTSESTAALGCLVKDYFPEPVPVTVS
WNSGALTSGVHTFPAVLQSSGLYSLSSVVTVPSSSLGTKTYTCNVDHKPSNTKVDKRVES
KYGPPCPPCPAPEFEGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGKAS
Anti-ASGPR1H11K-LV-var2-hIgGK-C (SEQ ID NO: 24):
ATGGAATCACAGACTCTGGTCTTCATATCCATACTGCTCTGGTTATATGGTGCTGATGG
GAACATTGTAATGACTCAATCTCCCAAATCCATGTCCATGTCAGTAGGGGAGAGGGTC
ACCTTGAGCTGCAAGGCCAGTGAGAATGTGGGAACTTATGTATCCTGGTATCAACAGA
GACCAGAACAGTCTCCAAAACTGCTGATATACGGGGCATCCAACCGGTACACTGGGGT
CCCCGATCGCTTCACAGGCAGTGGATCTGCAACAGATTTCACTCTGACCATCAGCAGT
GTGCAGGCTGAGGACCTTGCAGATTATCACTGTGGACAGACTTACAGCTATATATTCA
CGTTCGGCTCGGGGACAAAGCTCGAGATCAAACGAACTGTGGCTGCACCATCTGTCTT
CATCTTCCCGCCATCTGATGAGCAGTTGAAATCTGGAACTGCCTCTGTTGTGTGCCTGC
TGAATAACTTCTATCCCAGAGAGGCCAAAGTACAGTGGAAGGTGGATAACGCCCTCCA
ATCGGGTAACTCCCAGGAGAGTGTCACAGAGCAGGACAGCAAGGACAGCACCTACAG
CCTCAGCAGCACCCTGACGCTGAGCAAAGCAGACTACGAGAAACACAAAGTCTATGC
CTGCGAAGTCACCCATCAGGGCCTGAGCTCGCCCGTCACAAAGAGCTTCAACAGGGG
AGAGTGTTAG
Anti-ASGPR1H11K-LV-var2-hIgGK-C (SEQ ID NO: 25):
METHSQVFVYMLLWLSGVEGNIVMTQSPKSMSMSVGERVTLSCKASENVGTYVSWYQQ
RPEQSPKLLIYGASNRYTGVPDRFTGSGSATDFTLTISSVQAEDLADYHCGQTYSYIFTFGS
GTKLEIKRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC
Anti-CD1d_2B5.3G10_H-V-hIgG4H-C (SEQ ID NO: 26):
ATGGGATGGAGCCGGATCTTTCTCTTCCTCCTGTCAATAACTGCAGGTGTCCATTGCCA
GGTCCAGGTGCAGCAGTCGGGACCTGAGTTGGTGAAGCCTGGGGCCTCAGTGAAGATT
TCCTGCAAAGCCTCTGGCGACGCATTCAGTAGTTCTTGGATGAACTGGGTGAAGCAGA
GGCCTGGACAGGGTCTTGAGTGGATTGGACGGATTTATCTTGGAGATGGAGATATTAA
TTACAATGGGAAGTTCAAGGGCAGGGCCACACTGACTGCAGACAAATCCTCCAGCAC
AGCCTACATGCAGCTCAGCAGCCTGACCTCTGTGGACTCTGCGGTCTATTTCTGCGCGA
GGCAGCTCGGGCTATGGTATGTTATGGACTACTGGGGTCAAGGAACCTCAGTCACCGT
CTCCTCAGCCAAAACAAAGGGCCCATCCGTCTTCCCCCTGGCGCCCTGCTCCAGGAGC
ACCTCCGAGAGCACAGCCGCCCTGGGCTGCCTGGTCAAGGACTACTTCCCCGAACCGG
TGACGGTGTCGTGGAACTCAGGCGCCCTGACCAGCGGCGTGCACACCTTCCCGGCTGT
CCTACAGTCCTCAGGACTCTACTCCCTCAGCAGCGTGGTGACCGTGCCCTCCAGCAGC
TTGGGCACGAAGACCTACACCTGCAACGTAGATCACAAGCCCAGCAACACCAAGGTG
GACAAGAGAGTTGAGTCCAAATATGGTCCCCCATGCCCACCCTGCCCAGCACCTGAGT
TCGAAGGGGGACCATCAGTCTTCCTGTTCCCCCCAAAACCCAAGGACACTCTCATGAT
CTCCCGGACCCCTGAGGTCACGTGCGTGGTGGTGGACGTGAGCCAGGAAGACCCCGA
GGTCCAGTTCAACTGGTACGTGGATGGCGTGGAGGTGCATAATGCCAAGACAAAGCC
GCGGGAGGAGCAGTTCAACAGCACGTACCGTGTGGTCAGCGTCCTCACCGTCCTGCAC
CAGGACTGGCTGAACGGCAAGGAGTACAAGTGCAAGGTCTCCAACAAAGGCCTCCCG
TCCTCCATCGAGAAAACCATCTCCAAAGCCAAAGGGCAGCCCCGAGAGCCACAGGTG
TACACCCTGCCCCCATCCCAGGAGGAGATGACCAAGAACCAGGTCAGCCTGACCTGCC
TGGTCAAAGGCTTCTACCCCAGCGACATCGCCGTGGAGTGGGAGAGCAATGGGCAGC
CGGAGAACAACTACAAGACCACGCCTCCCGTGCTGGACTCCGACGGCTCCTTCTTCCT
CTACAGCAGGCTAACCGTGGACAAGAGCAGGTGGCAGGAGGGGAATGTCTTCTCATG
CTCCGTGATGCATGAGGCTCTGCACAACCACTACACACAGAAGAGCCTCTCCCTGTCT
CTGGGTAAAGCTAGCTGA
Anti-CD1d_2B5.3G10_H-V-hIgG4H-C (SEQ ID NO: 27):
MGWSRIFLFLLSITAGVHCQVQVQQSGPELVKPGASVKISCKASGDAFSSSWMNWVKQRP
GQGLEWIGRIYLGDGDINYNGKFKGRATLTADKSSSTAYMQLSSLTSVDSAVYFCARQLG
LWYVMDYWGQGTSVTVSSAKTKGPSVFPLAPCSRSTSESTAALGCLVKDYFPEPVTVSW
NSGALTSGVHTFPAVLQSSGLYSLSSVVTVPSSSLGTKTYTCNVDHKPSNTKVDKRVESKY
GPPCPPCPAPEFEGGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYKTTPPVLDSDGSF
FLYSRLTVDKSRWQEGNVFSCSVMHEALHNHYTQKSLSLSLGKAS
Anti-CD1d_2B5.3G10_K-V-hIgGK-C (SEQ ID NO: 28):
ATGAGTGTGCCCACTCAGGTCCTGGGGTTGCTGCTGCTGTGGCTTACAGGTGCCAGAT
GTGACATCCAGATGGCTCAGTCTCCAGCCTCCCTATCTGCATCTGTGGGAGAAACTGT
CACCATCACATGTCGAGCAAGTGAGAATATTTACAGTTATTTAGCATGGTATCAGCAG
AAACAGGGAAAATCTCCTCAGCTCCTGGTCTATAATGCAAAAACCTTAGCAGAAGGTG
TGCCATCAAGGTTCAGTGGCAGTGGATCAGGCACACAGTTTTCTCTGAAGATCAACAG
CCTGCAGCCTGAAGATTTTGGGAGTTATTACTGTCAACATCATTATGGTTTTCCGTGGA
CGTTCGGTGGAGGCACCAAGCTCGAGATCAAACGAACTGTGGCTGCACCATCTGTCTT
CATCTTCCCGCCATCTGATGAGCAGTTGAAATCTGGAACTGCCTCTGTTGTGTGCCTGC
TGAATAACTTCTATCCCAGAGAGGCCAAAGTACAGTGGAAGGTGGATAACGCCCTCCA
ATCGGGTAACTCCCAGGAGAGTGTCACAGAGCAGGACAGCAAGGACAGCACCTACAG
CCTCAGCAGCACCCTGACGCTGAGCAAAGCAGACTACGAGAAACACAAAGTCTATGC
CTGCGAAGTCACCCATCAGGGCCTGAGCTCGCCCGTCACAAAGAGCTTCAACAGGGG
AGAGTGTTAG
Anti-CD1d_2B5.3G10_K-V-hIgGK-C (SEQ ID NO: 29):
MSVPTQVLGLLLLWLTGARCDIQMAQSPASLSASVGETVTITCRASENIYSYLAWYQQKQ
GKSPQLLVYNAKTLAEGVPSRFSGSGSGTQFSLKINSLQPEDFGSYYCQHHYGFPWTFGGG
TKLEIKRTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC
Anti-CD1d_2H11.2G5_H-V-hIgG4H-C (SEQ ID NO: 30):
ATGAACTTCGGGCTCAGCTTGATTTTCCTTGTCCTCATTTTAAAAGGTGTCCAGTGTGA
GGTGCAGCTGGTGGAGTCTGGGGGAGACTTAGTGAAGCCTGGAGGGTCCCTGAAACT
CTCCTGTGCAGCCTCTGGATTCACTTTCAGTAGCTATGGCATGTCTTGGGTTCGCCAGA
CTCCAGACAAGAGGCTGGAGTGGGTCGCAGTCATTAGTAGTGGTGGAAGTTCCACCTT
CTATCCAGACAGTGTGAAGGGGCGATTCACCATCTCCAGAGACAATGCCAAGAACAC
CCTGTACCTGCAAATGAGCAGTCTGAAGTCTGAGGACACAGCCGTGTATTACTGTTCA
AGAGGAGGTTACTACTTTGACTACTGGGGCCAAGGCACCACTCTCACAGTCTCCGCAG
CCAAAACAAAGGGCCCATCCGTCTTCCCCCTGGCGCCCTGCTCCAGGAGCACCTCCGA
GAGCACAGCCGCCCTGGGCTGCCTGGTCAAGGACTACTTCCCCGAACCGGTGACGGTG
TCGTGGAACTCAGGCGCCCTGACCAGCGGCGTGCACACCTTCCCGGCTGTCCTACAGT
CCTCAGGACTCTACTCCCTCAGCAGCGTGGTGACCGTGCCCTCCAGCAGCTTGGGCAC
GAAGACCTACACCTGCAACGTAGATCACAAGCCCAGCAACACCAAGGTGGACAAGAG
AGTTGAGTCCAAATATGGTCCCCCATGCCCACCCTGCCCAGCACCTGAGTTCGAAGGG
GGACCATCAGTCTTCCTGTTCCCCCCAAAACCCAAGGACACTCTCATGATCTCCCGGA
CCCCTGAGGTCACGTGCGTGGTGGTGGACGTGAGCCAGGAAGACCCCGAGGTCCAGTT
CAACTGGTACGTGGATGGCGTGGAGGTGCATAATGCCAAGACAAAGCCGCGGGAGGA
GCAGTTCAACAGCACGTACCGTGTGGTCAGCGTCCTCACCGTCCTGCACCAGGACTGG
CTGAACGGCAAGGAGTACAAGTGCAAGGTCTCCAACAAAGGCCTCCCGTCCTCCATCG
AGAAAACCATCTCCAAAGCCAAAGGGCAGCCCCGAGAGCCACAGGTGTACACCCTGC
CCCCATCCCAGGAGGAGATGACCAAGAACCAGGTCAGCCTGACCTGCCTGGTCAAAG
GCTTCTACCCCAGCGACATCGCCGTGGAGTGGGAGAGCAATGGGCAGCCGGAGAACA
ACTACAAGACCACGCCTCCCGTGCTGGACTCCGACGGCTCCTTCTTCCTCTACAGCAG
GCTAACCGTGGACAAGAGCAGGTGGCAGGAGGGGAATGTCTTCTCATGCTCCGTGATG
CATGAGGCTCTGCACAACCACTACACACAGAAGAGCCTCTCCCTGTCTCTGGGTAAAG
CTAGCTGA
Anti-CD1d_2H11.2G5_H-V-hIgG4H-C (SEQ ID NO: 31):
MNFGLSLIFLVLILKGVQCEVQLVESGGDLVKPGGSLKLSCAASGFTFSSYGMSWVRQTPD
KRLEWVAVISSGGSSTFYPDSVKGRFTISRDNAKNTLYLQMSSLKSEDTAVYYCSRGGYY
FDYWGQGTTLTVSAAKTKGPSVFPLAPCSRSTSESTAALGCLVKDYFPEPVTVSWNSGAL
TSGVHTFPAVLQSSGLYSLSSVVTVPSSSLGTKTYTCNVDHKPSNTKVDKRVESKYGPPCP
PCPAPEFEGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPREPQ
VYTLPPSQEEMTKNQVSLTCLVKGFYPSDIAVEWESNGQPENNYKTTPPVLDSDGSFFLYS
RLTVDKSRWQEGNVFSCSVMHEALHNHYTQKSLSLSLGKAS
Anti-CD1d_2H11.2G5_K-V-hIgGK-C (SEQ ID NO: 32):
ATGAGGTTCCAGGTTCAGGTTCTGGGGCTCCTTCTGCTCTGGATATCAGGTGCCCAGTG
TGATGTCCAGATAACCCAGTCTCCATCTTATCTTGCTGCATCTCCTGGAGAAACCATTA
CTATTAATTGCAGGGCAAGCAAGACCATTAGCAAATATTTAGCCTGGTATCAAGAGAA
ACCTGAGAAAACTGATAAGCTTCTTATCTACTCTGGATCCACTTTGCAATCTGGAATTC
CATCAAGGTTCAGTGGCAGTGGATCTGGTACAGATTTCACTCTCACCATCAGTGGCCT
GGAGCCTGAAGATTTTGCAATGTATTACTGTCAACAGCATAATGAATACCCGTGGACG
TTCGGTGGAGGCACCAAGCTCGAGATCAAACGAACTGTGGCTGCACCATCTGTCTTCA
TCTTCCCGCCATCTGATGAGCAGTTGAAATCTGGAACTGCCTCTGTTGTGTGCCTGCTG
AATAACTTCTATCCCAGAGAGGCCAAAGTACAGTGGAAGGTGGATAACGCCCTCCAAT
CGGGTAACTCCCAGGAGAGTGTCACAGAGCAGGACAGCAAGGACAGCACCTACAGCC
TCAGCAGCACCCTGACGCTGAGCAAAGCAGACTACGAGAAACACAAAGTCTATGCCT
GCGAAGTCACCCATCAGGGCCTGAGCTCGCCCGTCACAAAGAGCTTCAACAGGGGAG
AGTGTTAG
Anti-CD1d_2H11.2G5_K-V-hIgGK-C (SEQ ID NO: 33):
MRFQVQVLGLLLLWISGAQCDVQITQSPSYLAASPGETITINCRASKTISKYLAWYQEKPE
KTDKLLIYSGSTLQSGIPSRFSGSGSGTDFTLTISGLEPEDFAMYYCQQHNEYPWTFGGGTK
LEIKRTVAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
Anti-CD40_11B6.1C3_H-LV-hIgG4H-C (SEQ ID NO: 34):
ATGGGATGGAGCTGGATCTTTCTCTTTCTCCTGTCAGGAACTGCAGGTGTCCTCTCTGA
GGTCCAGCTGCAACAGTCTGGACCTGAGCTGGTGAAGCCTGGGGCTTCAGTGAAGATA
TCCTGCAAGGCTTCTGGTTACTCATTCACTGGCTACTACATGCACTGGGTGAAGCAAA
GCCATGTAAAGAGCCTTGAGTGGATTGGACGTATTAATCCTTACAATGGTGCTACTAG
CTACAACCAGAATTTCAAGGACAAGGCCAGCTTGACTGTAGATAAGTCCTCCAGCACA
GCCTACATGGAGCTCCACAGCCTGACATCTGAGGACTCTGCAGTCTATTACTGTGCAA
GAGAGGACTACGTCTACTGGGGCCAAGGCACCACTCTCACAGTCTCCTCAGCCAAAAC
GAAGGGCCCATCCGTCTTCCCCCTGGCGCCCTGCTCCAGGAGCACCTCCGAGAGCACA
GCCGCCCTGGGCTGCCTGGTCAAGGACTACTTCCCCGAACCGGTGACGGTGTCGTGGA
ACTCAGGCGCCCTGACCAGCGGCGTGCACACCTTCCCGGCTGTCCTACAGTCCTCAGG
ACTCTACTCCCTCAGCAGCGTGGTGACCGTGCCCTCCAGCAGCTTGGGCACGAAGACC
TACACCTGCAACGTAGATCACAAGCCCAGCAACACCAAGGTGGACAAGAGAGTTGAG
TCCAAATATGGTCCCCCATGCCCACCCTGCCCAGCACCTGAGTTCGAAGGGGGACCAT
CAGTCTTCCTGTTCCCCCCAAAACCCAAGGACACTCTCATGATCTCCCGGACCCCTGAG
GTCACGTGCGTGGTGGTGGACGTGAGCCAGGAAGACCCCGAGGTCCAGTTCAACTGGT
ACGTGGATGGCGTGGAGGTGCATAATGCCAAGACAAAGCCGCGGGAGGAGCAGTTCA
ACAGCACGTACCGTGTGGTCAGCGTCCTCACCGTCCTGCACCAGGACTGGCTGAACGG
CAAGGAGTACAAGTGCAAGGTCTCCAACAAAGGCCTCCCGTCCTCCATCGAGAAAAC
CATCTCCAAAGCCAAAGGGCAGCCCCGAGAGCCACAGGTGTACACCCTGCCCCCATCC
CAGGAGGAGATGACCAAGAACCAGGTCAGCCTGACCTGCCTGGTCAAAGGCTTCTAC
CCCAGCGACATCGCCGTGGAGTGGGAGAGCAATGGGCAGCCGGAGAACAACTACAAG
ACCACGCCTCCCGTGCTGGACTCCGACGGCTCCTTCTTCCTCTACAGCAGGCTAACCGT
GGACAAGAGCAGGTGGCAGGAGGGGAATGTCTTCTCATGCTCCGTGATGCATGAGGC
TCTGCACAACCACTACACACAGAAGAGCCTCTCCCTGTCTCTGGGTAAAGCTAGCTGA
Anti-CD40_11B6.1C3_H-LV-hIgG4H-C (SEQ ID NO: 35):
MGWSWIFLFLLSGTAGVLSEVQLQQSGPELVKPGASVKISCKASGYSFTGYYMHWVKQS
HVKSLEWIGRINPYNGATSYNQNFKDKASLTVDKSSSTAYMELHSLTSEDSAVYYCARED
YVYWGQGTTLTVSSAKTKGPSVFPLAPCSRSTSESTAALGCLVKDYFPEPVTVSWNSGAL
TSGVHTFPAVLQSSGLYSLSSVVTVPSSSLGTKTYTCNVDHKPSNTKVDKRVESKYGPPCP
PCPAPEFEGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPREPQ
VYTLPPSQEEMTKNQVSLTCLVKGFYPSDIAVEWESNGQPENNYKTTPPVLDSDGSFFLYS
RLTVDKSRWQEGNVFSCSVMHEALHNHYTQKSLSLSLGKAS
Anti-CD40_11B6.1C3_K-LV-hIgGK-C (SEQ ID NO: 36):
ATGAAGTTGCCTGTTAGGCTGTTGGTGCTGATGTTCTGGATTCCTGCTTCCAGCAGTGA
TGTTGTGATGACCCAAACTCCACTCTCCCTGCCTGTCAGTCTTGGAGATCAAGCCTCCA
TCTCTTGCAGATCTAGTCAGAGCCTTGTACACAGTAATGGAAACACCTATTTACATTGG
TACCTGCAGAAGCCAGGCCAGTCTCCAAAGCTCCTGATCTACAAAGTTTCCAACCGAT
TTTCTGGGGTCCCAGACAGGTTCAGTGGCAGTGGATCAGGGACAGATTTCGCACTCAA
GATCAGTAGAGTGGAGGCTGAGGATCTGGGAGTTTATTTCTGCTCTCAAAGTACACAT
GTTCCGTGGACGTTCGGTGGAGGCACCAAGCTCGAGATCAAACGAACTGTGGCTGCAC
CATCTGTCTTCATCTTCCCGCCATCTGATGAGCAGTTGAAATCTGGAACTGCCTCTGTT
GTGTGCCTGCTGAATAACTTCTATCCCAGAGAGGCCAAAGTACAGTGGAAGGTGGATA
ACGCCCTCCAATCGGGTAACTCCCAGGAGAGTGTCACAGAGCAGGACAGCAAGGACA
GCACCTACAGCCTCAGCAGCACCCTGACGCTGAGCAAAGCAGACTACGAGAAACACA
AAGTCTATGCCTGCGAAGTCACCCATCAGGGCCTGAGCTCGCCCGTCACAAAGAGCTT
CAACAGGGGAGAGTGTTAG
Anti-CD40_11B6.1C3_K-LV-hIgGK-C (SEQ ID NO: 37):
MKLPVRLLVLMFWIPASSSDVVMTQTPLSLPVSLGDQASISCRSSQSLVHSNGNTYLHWY
LQKPGQSPKLLIYKVSNRFSGVPDRFSGSGSGTDFALKISRVEAEDLGVYFCSQSTHVPWTF
GGGTKLEIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
Anti-CD40_12B4.2C10_H-LV-hIgG4H-C (SEQ ID NO: 38):
ATGGAATGGAGTTGGATATTTCTCTTTCTTCTGTCAGGAACTGCAGGTGTCCACTCTGA
GGTCCAGCTGCAGCAGTCTGGACCTGAGCTGGTAAAGCCTGGGGCTTCAGTGAAGATG
TCCTGCAAGGCTTCTGGATACACATTCACTGACTATGTTTTGCACTGGGTGAAACAGA
AGCCTGGGCAGGGCCTTGAGTGGATTGGATATATTAATCCTTACAATGATGGTACTAA
GTACAATGAGAAGTTCAAAGGCAAGGCCACACTGACTTCAGACAAATCCTCCAGCAC
AGCCTACATGGAGCTCAGCAGCCTGACCTCTGAGGACTCTGCGGTCTATTACTGTGCA
AGGGGCTATCCGGCCTACTCTGGGTATGCTATGGACTACTGGGGTCAAGGAACCTCAG
TCACCGTCTCCTCAGCCAAAACGAAGGGCCCATCCGTCTTCCCCCTGGCGCCCTGCTCC
AGGAGCACCTCCGAGAGCACAGCCGCCCTGGGCTGCCTGGTCAAGGACTACTTCCCCG
AACCGGTGACGGTGTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCCCTCCA
GCAGCTTGGGCACGAAGACCTACACCTGCAACGTAGATCACAAGCCCAGCAACACCA
AGGTGGACAAGAGAGTTGAGTCCAAATATGGTCCCCCATGCCCACCCTGCCCAGCACC
TGAGTTCGAAGGGGGACCATCAGTCTTCCTGTTCCCCCCAAAACCCAAGGACACTCTC
ATGATCTCCCGGACCCCTGAGGTCACGTGCGTGGTGGTGGACGTGAGCCAGGAAGACC
CCGAGGTCCAGTTCAACTGGTACGTGGATGGCGTGGAGGTGCATAATGCCAAGACAA
AGCCGCGGGAGGAGCAGTTCAACAGCACGTACCGTGTGGTCAGCGTCCTCACCGTCCT
GCACCAGGACTGGCTGAACGGCAAGGAGTACAAGTGCAAGGTCTCCAACAAAGGCCT
CCCGTCCTCCATCGAGAAAACCATCTCCAAAGCCAAAGGGCAGCCCCGAGAGCCACA
GGTGTACACCCTGCCCCCATCCCAGGAGGAGATGACCAAGAACCAGGTCAGCCTGAC
CTGCCTGGTCAAAGGCTTCTACCCCAGCGACATCGCCGTGGAGTGGGAGAGCAATGGG
CAGCCGGAGAACAACTACAAGACCACGCCTCCCGTGCTGGACTCCGACGGCTCCTTCT
TCCTCTACAGCAGGCTAACCGTGGACAAGAGCAGGTGGCAGGAGGGGAATGTCTTCTC
ATGCTCCGTGATGCATGAGGCTCTGCACAACCACTACACACAGAAGAGCCTCTCCCTG
TCTCTGGGTAAAGCTAGCTGA
Anti-CD40_12B4.2C10_H-LV-hIgG4H-C (SEQ ID NO: 39):
MEWSWIFLFLLSGTAGVHSEVQLQQSGPELVKPGASVKMSCKASGYTFTDYVLHWVKQK
PGQGLEWIGYINPYNDGTKYNEKFKGKATLTSDKSSSTAYMELSSLTSEDSAVYYCARGY
PAYSGYAMDYWGQGTSVTVSSAKTKGPSVFPLAPCSRSTSESTAALGCLVKDYFPEPVTV
SWNSGALTSGVHTFPAVLQSSGLYSLSSVVTVPSSSLGTKTYTCNVDHKPSNTKVDKRVES
KYGPPCPPCPAPEFEGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGKAS
Anti-CD40_12B4.2C10_K-LV-v2-hIgGK-C (SEQ ID NO: 40):
ATGATGTCCTCTGCTCAGTTCCTTGGTCTCCTGTTGCTCTGTTTTCAAGGTACCAGATGT
GATATCCAGATGACACAGACTACATCCTCCCTGTCTGCCTCTCTGGGAGACAGAGTCA
CCATCAGTTGCAGGGCAAGTCAGGACATTAGCAATTATTTAAACTGGTATCAGCAGAA
ACCAGATGGAACTGTTAAACTCCTGATCTACTACACATCAAGATTACACTCAGGAGTC
CCATCAAGGTTCAGTGGCAGTGGGTCTGGAACAGATTATTCTCTCACCATTAGCAACC
TGGAGCAAGAAGATATTGCCACTTACTTTTGCCATCATGGTAATACGCTTCCGTGGAC
GTTCGGTGGAGGCACCAAGCTCGAGATCAAACGAACTGTGGCTGCACCATCTGTCTTC
ATCTTCCCGCCATCTGATGAGCAGTTGAAATCTGGAACTGCCTCTGTTGTGTGCCTGCT
GAATAACTTCTATCCCAGAGAGGCCAAAGTACAGTGGAAGGTGGATAACGCCCTCCA
ATCGGGTAACTCCCAGGAGAGTGTCACAGAGCAGGACAGCAAGGACAGCACCTACAG
CCTCAGCAGCACCCTGACGCTGAGCAAAGCAGACTACGAGAAACACAAAGTCTATGC
CTGCGAAGTCACCCATCAGGGCCTGAGCTCGCCCGTCACAAAGAGCTTCAACAGGGG
AGAGTGTTAG
Anti-CD40_12B4.2C10_K-LV-v2-hIgGK-C (SEQ ID NO: 41):
MMSSAQFLGLLLLCFQGTRCDIQMTQTTSSLSASLGDRVTISCRASQDISNYLNWYQQKPD
GTVKLLIYYTSRLHSGVPSRFSGSGSGTDYSLTISNLEQEDIATYFCHHGNTLPWTFGGGTK
LEIKRTVAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
Anti-CD40_12E12.3F3_H-V-hIgG4H-C (SEQ ID NO: 42):
ATGAACTTGGGGCTCAGCTTGATTTTCCTTGTCCTTGTTTTAAAAGGTGTCCAGTGTGA
AGTGAAGCTGGTGGAGTCTGGGGGAGGCTTAGTGCAGCCTGGAGGGTCCCTGAAACT
CTCCTGTGCAACCTCTGGATTCACTTTCAGTGACTATTACATGTATTGGGTTCGCCAGA
CTCCAGAGAAGAGGCTGGAGTGGGTCGCATACATTAATTCTGGTGGTGGTAGCACCTA
TTATCCAGACACTGTAAAGGGCCGATTCACCATCTCCAGAGACAATGCCAAGAACACC
CTGTACCTGCAAATGAGCCGGCTGAAGTCTGAGGACACAGCCATGTATTACTGTGCAA
GACGGGGGTTACCGTTCCATGCTATGGACTATTGGGGTCAAGGAACCTCAGTCACCGT
CTCCTCAGCCAAAACGAAGGGCCCATCCGTCTTCCCCCTGGCGCCCTGCTCCAGGAGC
ACCTCCGAGAGCACAGCCGCCCTGGGCTGCCTGGTCAAGGACTACTTCCCCGAACCGG
TGACGGTGTCGTGGAACTCAGGCGCCCTGACCAGCGGCGTGCACACCTTCCCGGCTGT
CCTACAGTCCTCAGGACTCTACTCCCTCAGCAGCGTGGTGACCGTGCCCTCCAGCAGC
TTGGGCACGAAGACCTACACCTGCAACGTAGATCACAAGCCCAGCAACACCAAGGTG
GACAAGAGAGTTGAGTCCAAATATGGTCCCCCATGCCCACCCTGCCCAGCACCTGAGT
TCGAAGGGGGACCATCAGTCTTCCTGTTCCCCCCAAAACCCAAGGACACTCTCATGAT
CTCCCGGACCCCTGAGGTCACGTGCGTGGTGGTGGACGTGAGCCAGGAAGACCCCGA
GGTCCAGTTCAACTGGTACGTGGATGGCGTGGAGGTGCATAATGCCAAGACAAAGCC
GCGGGAGGAGCAGTTCAACAGCACGTACCGTGTGGTCAGCGTCCTCACCGTCCTGCAC
CAGGACTGGCTGAACGGCAAGGAGTACAAGTGCAAGGTCTCCAACAAAGGCCTCCCG
TCCTCCATCGAGAAAACCATCTCCAAAGCCAAAGGGCAGCCCCGAGAGCCACAGGTG
TACACCCTGCCCCCATCCCAGGAGGAGATGACCAAGAACCAGGTCAGCCTGACCTGCC
TGGTCAAAGGCTTCTACCCCAGCGACATCGCCGTGGAGTGGGAGAGCAATGGGCAGC
CGGAGAACAACTACAAGACCACGCCTCCCGTGCTGGACTCCGACGGCTCCTTCTTCCT
CTACAGCAGGCTAACCGTGGACAAGAGCAGGTGGCAGGAGGGGAATGTCTTCTCATG
CTCCGTGATGCATGAGGCTCTGCACAACCACTACACACAGAAGAGCCTCTCCCTGTCT
CTGGGTAAAGCTAGCTGA
Anti-CD40_12E12.3F3_H-V-hIgG4H-C (SEQ ID NO: 43):
MNLGLSLIFLVLVLKGVQCEVKLVESGGGLVQPGGSLKLSCATSGFTFSDYYMYWVRQTP
EKRLEWVAYINSGGGSTYYPDTVKGRFTISRDNAKNTLYLQMSRLKSEDTAMYYCARRG
LPFHAMDYWGQGTSVTVSSAKTKGPSVFPLAPCSRSTSESTAALGCLVKDYFPEPVTVSW
NSGALTSGVHTFPAVLQSSGLYSLSSVVTVPSSSLGTKTYTCNVDHKPSNTKVDKRVESKY
GPPCPPCPAPEFEGGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYKTTPPVLDSDGSF
FLYSRLTVDKSRWQEGNVFSCSVMHEALHNHYTQKSLSLSLGKAS
Anti-CD40_12E12.3F3_K-LV-hIgGK-C (SEQ ID NO: 44):
ATGATGTCCTCTGCTCAGTTCCTTGGTCTCCTGTTGCTCTGTTTTCAAGGTACCAGATGT
GATATCCAGATGACACAGACTACATCCTCCCTGTCTGCCTCTCTAGGAGACAGAGTCA
CCATCAGTTGCAGTGCAAGTCAGGGCATTAGCAATTATTTAAACTGGTATCAGCAGAA
ACCAGATGGAACTGTTAAACTCCTGATCTATTACACATCAATTTTACACTCAGGAGTCC
CATCAAGGTTCAGTGGCAGTGGGTCTGGGACAGATTATTCTCTCACCATCGGCAACCT
GGAACCTGAAGATATTGCCACTTACTATTGTCAGCAGTTTAATAAGCTTCCTCCGACGT
TCGGTGGAGGCACCAAACTCGAGATCAAACGAACTGTGGCTGCACCATCTGTCTTCAT
CTTCCCGCCATCTGATGAGCAGTTGAAATCTGGAACTGCCTCTGTTGTGTGCCTGCTGA
ATAACTTCTATCCCAGAGAGGCCAAAGTACAGTGGAAGGTGGATAACGCCCTCCAATC
GGGTAACTCCCAGGAGAGTGTCACAGAGCAGGACAGCAAGGACAGCACCTACAGCCT
CAGCAGCACCCTGACGCTGAGCAAAGCAGACTACGAGAAACACAAAGTCTATGCCTG
CGAAGTCACCCATCAGGGCCTGAGCTCGCCCGTCACAAAGAGCTTCAACAGGGGAGA
GTGTTAG
Anti-CD40_12E12.3F3_K-LV-hIgGK-C (SEQ ID NO: 45):
MMSSAQFLGLLLLCFQGTRCDIQMTQTTSSLSASLGDRVTISCSASQGISNYLNWYQQKPD
GTVKLLIYYTSILHSGVPSRFSGSGSGTDYSLTIGNLEPEDIATYYCQQFNKLPPTFGGGTKL
EIKRTVAAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
Anti-DCIR_24A5.4A5_H-V-hIgG4H-C (SEQ ID NO: 46):
ATGGATTGGCTGTGGAACTTGCTATTCCTGATGGCAGCTGCCCAAAGTGCCCAAGCAC
AGATCCAGTTGGTGCAGTCTGGACCTGAGCTGAAGAAGCCTGGAGAGACAGTCAAGA
TCTCCTGCAAGGCTTCTGGGTATTCCTTCACAAACTATGGAATGAACTGGGTGAAACA
GGCTCCAGGAAAGGGTTTAAAGTGGATGGGCTGGATAAACACCTACACTGGAGAGTC
AACATATGCTGATGACTTCAAGGGACGGTTTGCCTTCTCTTTGGAAACCTCTGCCAGCA
CTGCCTATTTGCAGATCAGTAACCTCAAAAATGAGGACATGGCTACATATTTCTGTGCT
AGAGGGGACTTTAGGTACTACTATTTTGACTACTGGGGCCAAGGCACCACTCTCACAG
GCTCCTCAGCCAAAACGAAGGGCCCATCCGTCTTCCCCCTGGCGCCCTGCTCCAGGAG
CACCTCCGAGAGCACAGCCGCCCTGGGCTGCCTGGTCAAGGACTACTTCCCCGAACCG
GTGACGGTGTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCAGCAG
CTTGGGCACGAAGACCTACACCTGCAACGTAGATCACAAGCCCAGCAACACCAAGGT
GGACAAGAGAGTTGAGTCCAAATATGGTCCCCCATGCCCACCCTGCCCAGCACCTGAG
TTCGAAGGGGGACCATCAGTCTTCCTGTTCCCCCCAAAACCCAAGGACACTCTCATGA
TCTCCCGGACCCCTGAGGTCACGTGCGTGGTGGTGGACGTGAGCCAGGAAGACCCCGA
GGTCCAGTTCAACTGGTACGTGGATGGCGTGGAGGTGCATAATGCCAAGACAAAGCC
GCGGGAGGAGCAGTTCAACAGCACGTACCGTGTGGTCAGCGTCCTCACCGTCCTGCAC
CAGGACTGGCTGAACGGCAAGGAGTACAAGTGCAAGGTCTCCAACAAAGGCCTCCCG
TCCTCCATCGAGAAAACCATCTCCAAAGCCAAAGGGCAGCCCCGAGAGCCACAGGTG
TACACCCTGCCCCCATCCCAGGAGGAGATGACCAAGAACCAGGTCAGCCTGACCTGCC
TGGTCAAAGGCTTCTACCCCAGCGACATCGCCGTGGAGTGGGAGAGCAATGGGCAGC
CGGAGAACAACTACAAGACCACGCCTCCCGTGCTGGACTCCGACGGCTCCTTCTTCCT
CTACAGCAGGCTAACCGTGGACAAGAGCAGGTGGCAGGAGGGGAATGTCTTCTCATG
CTCCGTGATGCATGAGGCTCTGCACAACCACTACACACAGAAGAGCCTCTCCCTGTCT
CTGGGTAAAGCTAGCTGAT
Anti-DCIR_24A5.4A5_H-V-hIgG4H-C (SEQ ID NO: 47):
MDWLWNLLFLMAAAQSAQAQIQLVQSGPELKKPGETVKISCKASGYSFTNYGMNWVKQ
APGKGLKWMGWINTYTGESTYADDFKGRFAFSLETSASTAYLQISNLKNEDMATYFCAR
GDFRYYYFDYWGQGTTLTGSSAKTKGPSVFPLAPCSRSTSESTAALGCLVKDYFPEPVTVS
WNSGALTSGVHTFPAVLQSSGLYSLSSVVTVPSSSLGTKTYTCNVDHKPSNTKVDKRVES
KYGPPCPPCPAPEFEGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGKAS
Anti-DCIR_24A5.4A5_K-V-hIgGK-C (SEQ ID NO: 48):
ATGAGTGTGCTCACTCAGGTCCTGGCGTTGCTGCTGCTGTGGCTTACAGGTGCCAGAT
GTGACATCCAGATGACTCAGTCTCCAGCCTCCCTATCTGCATCTGTGGGAGAAACTGT
CACCATCACGTGTCGAGCAAGTGGGAATATTCACAATTATTTAGCATGGTATCAGCAG
AAACAGGGAAAATCTCCTCAGCTCCTGGTCTATAATGCAAAAACCTTGGCAGATGGTG
TGCCATCAAGGTTCAGTGGCAGTGGATCAGGAACACAATATTCTCTCAAGATCAACAC
CCTGCAGCCTGAAGATTTTGGGAGTTATTACTGTCAACATTTTTGGGATTCTTGGACGT
TCGGTGGAGGCACCAAGCTCGAGATCAAACGAACTGTGGCTGCACCATCTGTCTTCAT
CTTCCCGCCATCTGATGAGCAGTTGAAATCTGGAACTGCCTCTGTTGTGTGCCTGCTGA
ATAACTTCTATCCCAGAGAGGCCAAAGTACAGTGGAAGGTGGATAACGCCCTCCAATC
GGGTAACTCCCAGGAGAGTGTCACAGAGCAGGACAGCAAGGACAGCACCTACAGCCT
CAGCAGCACCCTGACGCTGAGCAAAGCAGACTACGAGAAACACAAAGTCTATGCCTG
CGAAGTCACCCATCAGGGCCTGAGCTCGCCCGTCACAAAGAGCTTCAACAGGGGAGA
GTGTTAG
Anti-DCIR_24A5.4A5_K-V-hIgGK-C (SEQ ID NO: 49):
MSVLTQVLALLLLWLTGARCDIQMTQSPASLSASVGETVTITCRASGNIHNYLAWYQQKQ
GKSPQLLVYNAKTLADGVPSRFSGSGSGTQYSLKINTLQPEDFGSYYCQHFWDSWTFGGG
TKLEIKRTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC
Anti-DCIR_24E7.3H9_H-V-hIgG4H-C (SEQ ID NO: 50):
ATGGAATGGACCTGGGTCTTTCTCTTCCTCCTGTCAGTAACTGCAGGTGTCCACTCCCA
GGTTCAGCTGCAGCAGTCTGGAGCTGAGCTGATGAAGCCTGGGGCCTCAGTGAAGATA
TCCTGCAAGGCTACTGGCTACACATTCAGTAGCTACTGGATAGAGTGGGTAAAGCAGA
GGCCTGGACATGGCCTTGAGTGGATTGGAGAGATTTTACCTGGAAGTGGTAGGACTAA
CGACAATGAGAAGTTCAAGGGCAAGGCCACATTCACTGCAGATACATCCTCCAAGAA
AGCCTACATGCAACTCAGCAGCCTGACATCTGAGGACTCTGCCGTCTATTATTGTGCA
AGAAGGGGTGGTTACTCCTTTGCTTACTGGGGCCAAGGGACTCTGGTCACTGTCTCTG
CAGCCAAAACAAAGGGCCCATCCGTCTTCCCCCTGGCGCCCTGCTCCAGGAGCACCTC
CGAGAGCACAGCCGCCCTGGGCTGCCTGGTCAAGGACTACTTCCCCGAACCGGTGACG
GTGTCGTGGAACTCAGGCGCCCTGACCAGCGGCGTGCACACCTTCCCGGCTGTCCTAC
AGTCCTCAGGACTCTACTCCCTCAGCAGCGTGGTGACCGTGCCCTCCAGCAGCTTGGG
CACGAAGACCTACACCTGCAACGTAGATCACAAGCCCAGCAACACCAAGGTGGACAA
GAGAGTTGAGTCCAAATATGGTCCCCCATGCCCACCCTGCCCAGCACCTGAGTTCGAA
GGGGGACCATCAGTCTTCCTGTTCCCCCCAAAACCCAAGGACACTCTCATGATCTCCC
GGACCCCTGAGGTCACGTGCGTGGTGGTGGACGTGAGCCAGGAAGACCCCGAGGTCC
AGTTCAACTGGTACGTGGATGGCGTGGAGGTGCATAATGCCAAGACAAAGCCGCGGG
AGGAGCAGTTCAACAGCACGTACCGTGTGGTCAGCGTCCTCACCGTCCTGCACCAGGA
CTGGCTGAACGGCAAGGAGTACAAGTGCAAGGTCTCCAACAAAGGCCTCCCGTCCTCC
ATCGAGAAAACCATCTCCAAAGCCAAAGGGCAGCCCCGAGAGCCACAGGTGTACACC
CTGCCCCCATCCCAGGAGGAGATGACCAAGAACCAGGTCAGCCTGACCTGCCTGGTCA
AAGGCTTCTACCCCAGCGACATCGCCGTGGAGTGGGAGAGCAATGGGCAGCCGGAGA
ACAACTACAAGACCACGCCTCCCGTGCTGGACTCCGACGGCTCCTTCTTCCTCTACAGC
AGGCTAACCGTGGACAAGAGCAGGTGGCAGGAGGGGAATGTCTTCTCATGCTCCGTG
ATGCATGAGGCTCTGCACAACCACTACACACAGAAGAGCCTCTCCCTGTCTCTGGGTA
AAGCTAGCTGA
Anti-DCIR_24E7.3H9_H-V-hIgG4H-C (SEQ ID NO: 51):
MEWTWVFLFLLSVTAGVHSQVQLQQSGAELMKPGASVKISCKATGYTFSSYWIEWVKQR
PGHGLEWIGEILPGSGRTNDNEKFKGKATFTADTSSKKAYMQLSSLTSEDSAVYYCARRG
GYSFAYWGQGTLVTVSAAKTKGPSVFPLAPCSRSTSESTAALGCLVKDYFPEPVTVSWNS
GALTSGVHTFPAVLQSSGLYSLSSVVTVPSSSLGTKTYTCNVDHKPSNTKVDKRVESKYGP
PCPPCPAPEFEGGPSVFLFPPKPKDTLMISRTPEVTCVVVDVSQEDPEVQFNWYVDGVEVH
NAKTKPREEQFNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTPPVLDSDGSFFL
YSRLTVDKSRWQEGNVFSCSVMHEALHNHYTQKSLSLSLGKAS
Anti-DCIR_24E7.3H9_K-V-hIgGK-C (SEQ ID NO: 52):
ATGACCATGTTCTCACTAGCTCTTCTCCTCAGTCTTCTTCTCCTCTGTGTCTCTGATTCT
AGGGCAGAAACAACTGTGACCCAGTCTATGACCATGTTCTCACTAGCTCTTCTCCTCA
GTCTTCTTCTCCTCTGTGTCTCTGATTCTAGGGCAGAAACAACTGTGACCCAGTCTCCA
GCATCCCTGTCCATGGCTATAGGGGAAAAAGTCACCATCAGATGCGTAACCAGCACTG
ATATTGATGATGATGTGAACTGGTACCAGCAGAAGCCAGGGGAACCTCCTAAACTCCT
TATTTCAGAAGGCAATACTCTTCGTCCTGGAGTCCCATCCCGATTCTCCAGCAGTGGCT
ATGGTACAGATTTTGTTTTTACAATTGAGAACATGCTCTCAGAAGATGTTGCAGATTAC
TACTGTTTGCAAAGTGGTAACTTGCCGTACACGTTCGGAGGGGGGACCAAGCTCGAGA
TCAAACGAACTGTGGCTGCACCATCTGTCTTCATCTTCCCGCCATCTGATGAGCAGTTG
AAATCTGGAACTGCCTCTGTTGTGTGCCTGCTGAATAACTTCTATCCCAGAGAGGCCA
AAGTACAGTGGAAGGTGGATAACGCCCTCCAATCGGGTAACTCCCAGGAGAGTGTCA
CAGAGCAGGACAGCAAGGACAGCACCTACAGCCTCAGCAGCACCCTGACGCTGAGCA
AAGCAGACTACGAGAAACACAAAGTCTATGCCTGCGAAGTCACCCATCAGGGCCTGA
GCTCGCCCGTCACAAAGAGCTTCAACAGGGGAGAGTGTTAGCCAGCATCCCTGTCCAT
GGCTATAGGGGAAAAAGTCACCATCAGATGCGTAACCAGCACTGATATTGATGATGAT
GTGAACTGGTACCAGCAGAAGCCAGGGGAACCTCCTAAACTCCTTATTTCAGAAGGCA
ATACTCTTCGTCCTGGAGTCCCATCCCGATTCTCCAGCAGTGGCTATGGTACAGATTTT
GTTTTTACAATTGAGAACATGCTCTCAGAAGATGTTGCAGATTACTACTGTTTGCAAAG
TGGTAACTTGCCGTACACGTTCGGAGGGGGGACCAAGCTCGAGATCAAACGAACTGT
GGCTGCACCATCTGTCTTCATCTTCCCGCCATCTGATGAGCAGTTGAAATCTGGAACTG
CCTCTGTTGTGTGCCTGCTGAATAACTTCTATCCCAGAGAGGCCAAAGTACAGTGGAA
GGTGGATAACGCCCTCCAATCGGGTAACTCCCAGGAGAGTGTCACAGAGCAGGACAG
CAAGGACAGCACCTACAGCCTCAGCAGCACCCTGACGCTGAGCAAAGCAGACTACGA
GAAACACAAAGTCTATGCCTGCGAAGTCACCCATCAGGGCCTGAGCTCGCCCGTCACA
AAGAGCTTCAACAGGGGAGAGTGTTAG
Anti-DCIR_24E7.3H9K-V-hIgGK-C (SEQ ID NO: 53):
MTMFSLALLLSLLLLCVSDSRAETTVTQSPASLSMAIGEKVTIRCVTSTDIDDDVNWYQQK
PGEPPKLLISEGNTLRPGVPSRFSSSGYGTDFVFTIENMLSEDVADYYCLQSGNLPYTFGGG
TKLEIKRTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC
Anti-DCIR_29E9.2E2_H-VhIgG4H-C (SEQ ID NO: 54):
ATGGCTTGGGTGTGGACCTTGCTATTCCTGATGGCAGCTGCCCAAAGTGCCCAAGCAC
AGATCCAGTTGGTGCAGTCTGGACCTGAGCTGAAGAAGCCTGGAGAGACAGTCAAGA
TCTCCTGCAAGGCTTCTGGGTATACCTTCACAAACTATGGAATGAACTGGGTGAAGCA
GGCTCCAGGAAAGGGTTTAAAGTGGGTGGGCTGGATAAACACCTTCACTGGAGAGCC
AACATATGTTGATGACTTCAAGGGACGGTTTGCCTTCTCTTTGGAAACCTCTGCCAGCA
CTGCCTATTTGCAGATCAACAACCTCAAAAATGAGGACACGGCTACATATTTCTGTGC
AAGAGGGAATTTTAGGTACTACTACTTTGACTACTGGGGCCAAGGCACCACTCTCACA
GTCTCCTCAGCCAAAACAAAGGGCCCATCCGTCTTCCCCCTGGCGCCCTGCTCCAGGA
GCACCTCCGAGAGCACAGCCGCCCTGGGCTGCCTGGTCAAGGACTACTTCCCCGAACC
GGTGACGGTGTCGTGGAACTCAGGCGCCCTGACCAGCGGCGTGCACACCTTCCCGGCT
GTCCTACAGTCCTCAGGACTCTACTCCCTCAGCAGCGTGGTGACCGTGCCCTCCAGCA
GCTTGGGCACGAAGACCTACACCTGCAACGTAGATCACAAGCCCAGCAACACCAAGG
TGGACAAGAGAGTTGAGTCCAAATATGGTCCCCCATGCCCACCCTGCCCAGCACCTGA
GTTCGAAGGGGGACCATCAGTCTTCCTGTTCCCCCCAAAACCCAAGGACACTCTCATG
ATCTCCCGGACCCCTGAGGTCACGTGCGTGGTGGTGGACGTGAGCCAGGAAGACCCCG
AGGTCCAGTTCAACTGGTACGTGGATGGCGTGGAGGTGCATAATGCCAAGACAAAGC
CGCGGGAGGAGCAGTTCAACAGCACGTACCGTGTGGTCAGCGTCCTCACCGTCCTGCA
CCAGGACTGGCTGAACGGCAAGGAGTACAAGTGCAAGGTCTCCAACAAAGGCCTCCC
GTCCTCCATCGAGAAAACCATCTCCAAAGCCAAAGGGCAGCCCCGAGAGCCACAGGT
GTACACCCTGCCCCCATCCCAGGAGGAGATGACCAAGAACCAGGTCAGCCTGACCTGC
CTGGTCAAAGGCTTCTACCCCAGCGACATCGCCGTGGAGTGGGAGAGCAATGGGCAG
CCGGAGAACAACTACAAGACCACGCCTCCCGTGCTGGACTCCGACGGCTCCTTCTTCC
TCTACAGCAGGCTAACCGTGGACAAGAGCAGGTGGCAGGAGGGGAATGTCTTCTCAT
GCTCCGTGATGCATGAGGCTCTGCACAACCACTACACACAGAAGAGCCTCTCCCTGTC
TCTGGGTAAAGCTAGCTGA
Anti-DCIR_29E9.2E2_H-VhIgG4H-C (SEQ ID NO: 55):
MAWVWTLLFLMAAAQSAQAQIQLVQSGPELKKPGETVKISCKASGYTFTNYGMNWVKQ
APGKGLKWVGWINTFTGEPTYVDDFKGRFAFSLETSASTAYLQINNLKNEDTATYFCARG
NFRYYYFDYWGQGTTLTVSSAKTKGPSVFPLAPCSRSTSESTAALGCLVKDYFPEPVTVS
WNSGALTSGVHTFPAVLQSSGLYSLSSVVTVPSSSLGTKTYTCNVDHKPSNTKVDKRVES
KYGPPCPPCPAPEFEGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGKAS
Anti-DCIR_29E9.2E2_K-V-hIgGK-C (SEQ ID NO: 56):
ATGAGTGTGCTCACTCAGGTCCTGGCGTTGCTGCTGCTGTGGCTTACAGGTGCCAGAT
GTGACATCCAGATGACTCAGTCCCCAGCCTCCCTATCTGCATCTGTGGGAGAAACTGT
CACCATCACATGTCGAACAAGTGGGAATATTCGCAATTATTTAGCATGGTATCAGCAG
AAACAGGGAAAATCTCCTCAACTCCTGGTCTATAATGCAAAAACCTTAGCAGATGGTG
TGCCATCAAGGTTCGGTGGCAGTGGATCAGGAACACAATATTCTCTCAAGATCAACAG
CCTGCAGCCTGAAGATTTTGGGAATTATTACTGTCAACATTTTTGGAGTAGTCCGTACA
CGTTCGGAGGGGGGACCAAGCTCGAGATCAAACGAACTGTGGCTGCACCATCTGTCTT
CATCTTCCCGCCATCTGATGAGCAGTTGAAATCTGGAACTGCCTCTGTTGTGTGCCTGC
TGAATAACTTCTATCCCAGAGAGGCCAAAGTACAGTGGAAGGTGGATAACGCCCTCCA
ATCGGGTAACTCCCAGGAGAGTGTCACAGAGCAGGACAGCAAGGACAGCACCTACAG
CCTCAGCAGCACCCTGACGCTGAGCAAAGCAGACTACGAGAAACACAAAGTCTATGC
CTGCGAAGTCACCCATCAGGGCCTGAGCTCGCCCGTCACAAAGAGCTTCAACAGGGG
AGAGTGTTAG
Anti-DCIR_29E9.2E2_K-V-hIgGK-C (SEQ ID NO: 57):
MSVLTQVLALLLLWLTGARCDIQMTQSPASLSASVGETVTITCRTSGNIRNYLAWYQQKQ
GKSPQLLVYNAKTLADGVPSRFGGSGSGTQYSLKINSLQPEDFGNYYCQHFWSSPYTFGG
GTKLEIKRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC
Anti-DCIR_29G10.3D9_H-V-hIgG4H-C (SEQ ID NO: 58):
ATGATGGGATGGAGCTATATCATCCTCTTTTTGGTAGCAACAGCTACAGATGTCCACTC
CCAGGTCCAACTGCAGCAGCCTGGGGCTGAACTGGTGAAGCCTGGGGCTTCAGTGAA
GCTGTCCTGCAAGGCTTCTGGCTACACCTTCACCAGCTACTGGATGCACTGGGTGAAG
CAGAGGCCTGGAGAAGGCCTTGAGTGGATTGGAGAGATTAATCCTAGCTACGGTCGTA
CTGACTACAATGAGAAGTTCAAGAACAAGGCCACACTGACTGTAGCCAAATCCTCCAG
CACAGCCTACATGCAACTCAGCAGCCTGACATCTGAGGACTCTGCGGTCTATTACTGT
GCAAGAGGAGATTACTACGGTAGTAGCTCGTTTGCTTACTGGGGCCAAGGGACTCTGG
TCACTGTCTCTGCAGCCAAAACAAAGGGCCCATCCGTCTTCCCCCTGGCGCCCTGCTCC
AGGAGCACCTCCGAGAGCACAGCCGCCCTGGGCTGCCTGGTCAAGGACTACTTCCCCG
AACCGGTGACGGTGTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCCCTCCA
GCAGCTTGGGCACGAAGACCTACACCTGCAACGTAGATCACAAGCCCAGCAACACCA
AGGTGGACAAGAGAGTTGAGTCCAAATATGGTCCCCCATGCCCACCCTGCCCAGCACC
TGAGTTCGAAGGGGGACCATCAGTCTTCCTGTTCCCCCCAAAACCCAAGGACACTCTC
ATGATCTCCCGGACCCCTGAGGTCACGTGCGTGGTGGTGGACGTGAGCCAGGAAGACC
CCGAGGTCCAGTTCAACTGGTACGTGGATGGCGTGGAGGTGCATAATGCCAAGACAA
AGCCGCGGGAGGAGCAGTTCAACAGCACGTACCGTGTGGTCAGCGTCCTCACCGTCCT
GCACCAGGACTGGCTGAACGGCAAGGAGTACAAGTGCAAGGTCTCCAACAAAGGCCT
CCCGTCCTCCATCGAGAAAACCATCTCCAAAGCCAAAGGGCAGCCCCGAGAGCCACA
GGTGTACACCCTGCCCCCATCCCAGGAGGAGATGACCAAGAACCAGGTCAGCCTGAC
CTGCCTGGTCAAAGGCTTCTACCCCAGCGACATCGCCGTGGAGTGGGAGAGCAATGGG
CAGCCGGAGAACAACTACAAGACCACGCCTCCCGTGCTGGACTCCGACGGCTCCTTCT
TCCTCTACAGCAGGCTAACCGTGGACAAGAGCAGGTGGCAGGAGGGGAATGTCTTCTC
ATGCTCCGTGATGCATGAGGCTCTGCACAACCACTACACACAGAAGAGCCTCTCCCTG
TCTCTGGGTAAAGCTAGCGGATGGAGCTATATCATCCTCTTTTTGGTAGCAACAGCTAC
AGATGTCCACTCCCAGGTCCAACTGCAGCAGCCTGGGGCTGAACTGGTGAAGCCTGGG
GCTTCAGTGAAGCTGTCCTGCAAGGCTTCTGGCTACACCTTCACCAGCTACTGGATGC
ACTGGGTGAAGCAGAGGCCTGGAGAAGGCCTTGAGTGGATTGGAGAGATTAATCCTA
GCTACGGTCGTACTGACTACAATGGGAAGTTCAAGAACAAGGCCACACTGACTGTAGC
CAAATCCTCCAGCACAGCCTACATGCAACTCAGCAGCCTGACATCTGAGGACTCTGCG
GTCTATTACTGTGCAAGAGGAGATTACTACGGTAGTAGCTCGTTTGCTTACTGGGGCC
AAGGGACTCTGGTCACTGTCTCTGCAGCCAAAACAAAGGGCCCATCCGTCTTCCCCCT
GGCGCCCTGCTCCAGGAGCACCTCCGAGAGCACAGCCGCCCTGGGCTGCCTGGTCAAG
GACTACTTCCCCGAACCGGTGACGGTGTCGTGGAACTCAGGCGCCCTGACCAGCGGCG
TGCACACCTTCCCGGCTGTCCTACAGTCCTCAGGACTCTACTCCCTCAGCAGCGTGGTG
ACCGTGCCCTCCAGCAGCTTGGGCACGAAGACCTACACCTGCAACGTAGATCACAAGC
CCAGCAACACCAAGGTGGACAAGAGAGTTGAGTCCAAATATGGTCCCCCATGCCCAC
CCTGCCCAGCACCTGAGTTCGAAGGGGGACCATCAGTCTTCCTGTTCCCCCCAAAACC
CAAGGACACTCTCATGATCTCCCGGACCCCTGAGGTCACGTGCGTGGTGGTGGACGTG
AGCCAGGAAGACCCCGAGGTCCAGTTCAACTGGTACGTGGATGGCGTGGAGGTGCAT
AATGCCAAGACAAAGCCGCGGGAGGAGCAGTTCAACAGCACGTACCGTGTGGTCAGC
GTCCTCACCGTCCTGCACCAGGACTGGCTGAACGGCAAGGAGTACAAGTGCAAGGTCT
CCAACAAAGGCCTCCCGTCCTCCATCGAGAAAACCATCTCCAAAGCCAAAGGGCAGC
CCCGAGAGCCACAGGTGTACACCCTGCCCCCATCCCAGGAGGAGATGACCAAGAACC
AGGTCAGCCTGACCTGCCTGGTCAAAGGCTTCTACCCCAGCGACATCGCCGTGGAGTG
GGAGAGCAATGGGCAGCCGGAGAACAACTACAAGACCACGCCTCCCGTGCTGGACTC
CGACGGCTCCTTCTTCCTCTACAGCAGGCTAACCGTGGACAAGAGCAGGTGGCAGGAG
GGGAATGTCTTCTCATGCTCCGTGATGCATGAGGCTCTGCACAACCACTACACACAGA
AGAGCCTCTCCCTGTCTCTGGGTAAAGCTAGCTGA
Anti-DCIR_29G10.3D9_H-V-hIgG4H-C (SEQ ID NO: 59):
MMGWSYIILFLVATATDVHSQVQLQQPGAELVKPGASVKLSCKASGYTFTSYWMHWVK
QRPGEGLEWIGEINPSYGRTDYNEKFKNKATLTVAKSSSTAYMQLSSLTSEDSAVYYCAR
GDYYGSSSFAYWGQGTLVTVSAAKTKGPSVFPLAPCSRSTSESTAALGCLVKDYFPEPVT
VSWNSGALTSGVHTFPAVLQSSGLYSLSSVVTVPSSSLGTKTYTCNVDHKPSNTKVDKRV
ESKYGPPCPPCPAPEFEGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIEKTISKA
KGQPREPQVYTLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGKAS
Anti-DCIR_29G10.3D9_K-Var1-V-hIgGK-C (SEQ ID NO: 60):
ATGGATTTTCAAGTGCAGATTTTCAGCTTCCTGCTAATGAGTGCCTCAGTCATAATGTC
CAGGGGACAAATTGTTCTCACCCAGTCTCCAGCACTCATGTCTGCATCTCCAGGGGAG
AAGGTCACCATGACCTGCAGTGCCAGCTCAAATATAAGTTACATGTACTGGTACCAGC
AGAAGCCAAGATCCTCCCCCAAACCCTGGATTTATCTCACATCCAACCTGGCTTCTGG
AGTCCCTGCTCGCTTCAGTGGCAGTGGGTCTGGGACCTCTTACTCTCTCACAACCAGCA
GCATGGAGGCTGAAGATGCTGCCACTTATTGCTGCCAGCAGTGGAGTAGTAACCCACC
CACGTTCGGTGCTGGGACCAAGCTCGAGATCAAACGAACTGTGGCTGCACCATCTGTC
TTCATCTTCCCGCCATCTGATGAGCAGTTGAAATCTGGAACTGCCTCTGTTGTGTGCCT
GCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTAC
AGCCTCAGCAGCACCCTGACGCTGAGCAAAGCAGACTACGAGAAACACAAAGTCTAT
GCCTGCGAAGTCACCCATCAGGGCCTGAGCTCGCCCGTCACAAAGAGCTTCAACAGGG
GAGAGTGTTAG
Anti-DCIR_29G10.3D9_K-Var1-V-hIgGK-C (SEQ ID NO: 61):
MDFQVQIFSFLLMSASVIMSRGQIVLTQSPALMSASPGEKVTMTCSASSNISYMYWYQQKP
RSSPKPWIYLTSNLASGVPARFSGSGSGTSYSLTTSSMEAEDAATYCCQQWSSNPPTFGAG
TKLEIKRTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC
Anti-DCIR_29G10.3D9_K-Var2-V-hIgGK-C (SEQ ID NO: 62):
ATGGATTTTCGAGTGCAGATTTTCAGCTTCCTGCTAATGAGTGCCTCAGTCATAATGTC
CAGGGGACAAATTGTTCTCACCCAGTCTCCAGCACTCATGTCTGCATCTCCAGGGGAG
AAGGTCACCATGACCTGCAGTGCCAGCTCAAATATAAGTTACATGTACTGGTACCAGC
AGAAGCCAAGATCCTCCCCCAAACCCTGGATTTATCTCACATCCAACCTGGCTTCTGG
AGTCCCTGCTCGCTTCAGTGGCAGTGGGTCTGGGACCTCTTACTCTCTCACAATCAGCA
GCATGGAGGCTGAAGATGCTGCCACTTATTACTGCCAGCAGTGGAGTAGTAACCCACC
CACGTTCGGTGCTGGGACCAAGCTCGAGATCAAACGAACTGTGGCTGCACCATCTGTC
TTCATCTTCCCGCCATCTGATGAGCAGTTGAAATCTGGAACTGCCTCTGTTGTGTGCCT
GCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTAC
AGCCTCAGCAGCACCCTGACGCTGAGCAAAGCAGACTACGAGAAACACAAAGTCTAT
GCCTGCGAAGTCACCCATCAGGGCCTGAGCTCGCCCGTCACAAAGAGCTTCAACAGGG
GAGAGTGTTAG
Anti-DCIR_29G10.3D9_K-Var2-V-hIgGK-C (SEQ ID NO: 63):
MDFRVQIFSFLLMSASVIMSRGQIVLTQSPALMSASPGEKVTMTCSASSNISYMYWYQQKP
RSSPKPWIYLTSNLASGVPARFSGSGSGTSYSLTISSMEAEDAATYYCQQWSSNPPTFGAGT
KLEIKRTVAAPSVFIFPPSDEQLKSGTASVVCLLNNFYPREAKVQWKVDNALQSGNSQESV
TEQDSKDSTYSLSSTLTLSKADYEKHKVYACEVTHQGLSSPVTKSFNRGEC
Anti-DCIR_2C9K-V-hIgGK-C (SEQ ID NO: 64):
ATGGAGACAGACACACTCCTGCTATGGGTGCTGCTGCTCTGGGTTCCAGGTTCCACAG
GTGACATTGTGCTGATCCAATCTCCAGCTTCTTTGGCTGTGTCTCTAGGGCAGAGGGCC
ACCATATCCTGCAGAGCCAGTGAAAGTGTTGATAGTTATGTCAATAGTTTTATGCACT
GGTACCAGCAGAAACCAGGACAGCCACCCAAACTCCTCATCTATCGTGTATCCAACCT
AGAATCTGGGATCCCTGCCAGGTTCAGTGGCAGTGGGTCTAGGACAGACTTCACCCTC
ACCATTAATCCTGTGGAGGCTGATGATGTTGCAACCTATTACTGTCAGCAAAGTAATG
AGGATCCATTCACGTTCGGCTCGGGGACAAAGCTCGAGATCAAACGAACTGTGGCTGC
ACCATCTGTCTTCATCTTCCCGCCATCTGATGAGCAGTTGAAATCTGGAACTGCCTCTG
TTGTGTGCCTGCTGAATAACTTCTATCCCAGAGAGGCCAAAGTACAGTGGAAGGTGGA
TAACGCCCTCCAATCGGGTAACTCCCAGGAGAGTGTCACAGAGCAGGACAGCAAGGA
CAGCACCTACAGCCTCAGCAGCACCCTGACGCTGAGCAAAGCAGACTACGAGAAACA
CAAAGTCTATGCCTGCGAAGTCACCCATCAGGGCCTGAGCTCGCCCGTCACAAAGAGC
TTCAACAGGGGAGAGTGTTAG
Anti-DCIR_2C9K-V-hIgGK-C (SEQ ID NO: 65):
METDTLLLWVLLLWVPGSTGDIVLIQSPASLAVSLGQRATISCRASESVDSYVNSFMHWY
QQKPGQPPKLLIYRVSNLESGIPARFSGSGSRTDFTLTINPVEADDVATYYCQQSNEDPFTF
GSGTKLEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC.
Anti-DCIR_31A6.1F5_H-var2-V-hIgG4H-C (SEQ ID NO: 66):
ATGGAATGTAACTGGATACTTCCTTTTATTCTGTCGGTAATTTCAGGGGTCTACTCAGA
GGTTCAGCTCCAGCAGTCTGGGACTGTGCTGGCAAGGCCTGGGGCTTCCGTGAATATG
TCCTGTAAGGCTGCTGGCTACAGCTTTACCAGTTACTGGGTGTACTGGGTCAAACAGA
GGCCTGGACAGGGTCTGGAATGGATTGGTGCTATTTACCCTAAAAATAGTAGAACTAG
CTACAACCAGAAGTTCCAGGACAAGGCCACACTGACTGCAGTCACATCCGCCAGCACT
GCCTACATGGAGCTCAGCAGCCTGACAAATGAGGACTCTGCGGTCTATTACTGTACAA
GACCTCACTATGATTCGTTTGGTTACTGGGGCCAAGGGACTCTGGTCACTGTCTCTGCA
GCCAAAACAaagggcccATCCGTCTTCCCCCTGGCGCCCTGCTCCAGGAGCACCTCCGAGA
GCACAGCCGCCCTGGGCTGCCTGGTCAAGGACTACTTCCCCGAACCGGTGACGGTGTC
GTGGAACTCAGGCGCCCTGACCAGCGGCGTGCACACCTTCCCGGCTGTCCTACAGTCC
TCAGGACTCTACTCCCTCAGCAGCGTGGTGACCGTGCCCTCCAGCAGCTTGGGCACGA
AGACCTACACCTGCAACGTAGATCACAAGCCCAGCAACACCAAGGTGGACAAGAGAG
TTGAGTCCAAATATGGTCCCCCATGCCCACCCTGCCCAGCACCTGAGTTCGAAGGGGG
ACCATCAGTCTTCCTGTTCCCCCCAAAACCCAAGGACACTCTCATGATCTCCCGGACCC
CTGAGGTCACGTGCGTGGTGGTGGACGTGAGCCAGGAAGACCCCGAGGTCCAGTTCA
ACTGGTACGTGGATGGCGTGGAGGTGCATAATGCCAAGACAAAGCCGCGGGAGGAGC
AGTTCAACAGCACGTACCGTGTGGTCAGCGTCCTCACCGTCCTGCACCAGGACTGGCT
GAACGGCAAGGAGTACAAGTGCAAGGTCTCCAACAAAGGCCTCCCGTCCTCCATCGA
GAAAACCATCTCCAAAGCCAAAGGGCAGCCCCGAGAGCCACAGGTGTACACCCTGCC
CCCATCCCAGGAGGAGATGACCAAGAACCAGGTCAGCCTGACCTGCCTGGTCAAAGG
CTTCTACCCCAGCGACATCGCCGTGGAGTGGGAGAGCAATGGGCAGCCGGAGAACAA
CTACAAGACCACGCCTCCCGTGCTGGACTCCGACGGCTCCTTCTTCCTCTACAGCAGGC
TAACCGTGGACAAGAGCAGGTGGCAGGAGGGGAATGTCTTCTCATGCTCCGTGATGCA
TGAGGCTCTGCACAACCACTACACACAGAAGAGCCTCTCCCTGTCTCTGGGTAAAGCT
AGCTGA
Anti-DCIR_31A6.1F5_H-var2-V-hIgG4H-C (SEQ ID NO: 67):
MECNWILPFILSVISGVYSEVQLQQSGTVLARPGASVNMSCKAAGYSFTSYWVYWVKQRP
GQGLEWIGAIYPKNSRTSYNQKFQDKATLTAVTSASTAYMELSSLTNEDSAVYYCTRPHY
DSFGYWGQGTLVTVSAAKTKGPSVFPLAPCSRSTSESTAALGCLVKDYFPEPVTVSWNSG
ALTSGVHTFPAVLQSSGLYSLSSVVTVPSSSLGTKTYTCNVDHKPSNTKVDKRVESKYGPP
CPPCPAPEFEGGPSVFLFPPKPKDTLMISRTPEVTCVVVDVSQEDPEVQFNWYVDGVEVHN
AKTKPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGSFFLY
SRLTVDKSRWQEGNVFSCSVMHEALHNHYTQKSLSLSLGKAS
Anti-DCIR_31A6.1F5_K-var2-V-hIgGK-C (SEQ ID NO: 68):
ATGGAGACAGACACACTCCTGCTATGGGTGCTGCTGCTCTGGGTTCCAGGTTCCACAG
GTGACATTGTGCTGACCCAATCTCCAGCTTCTTTGGCTGTGTCTCTAGGGCAGAGGGCC
ACCATATCCTGCAGAGCCAGTGAAAGTGTAGATAGTTATGGCATTAGTTTTATGCACT
GGTACCAGCAGAAACCAGGACAGCCACCCAAACTCCTCATCTATCGTGCATCCAACCA
AGAATCTGGGATCCCTGCCAGGTTCAGTGGCAGTGGGTCTAGGACAGACTTCACCCTC
ACCATTAATCCTGTGGAGGCTGATGATGTTGCAACCTATTACTGTCAGCAAAGTAATG
AGGATCCGCTCACGTTCGGTGCTGGGACCAAGCTCGAGATCAAACGAACTGTGGCTGC
ACCATCTGTCTTCATCTTCCCGCCATCTGATGAGCAGTTGAAATCTGGAACTGCCTCTG
TTGTGTGCCTGCTGAATAACTTCTATCCCAGAGAGGCCAAAGTACAGTGGAAGGTGGA
TAACGCCCTCCAATCGGGTAACTCCCAGGAGAGTGTCACAGAGCAGGACAGCAAGGA
CAGCACCTACAGCCTCAGCAGCACCCTGACGCTGAGCAAAGCAGACTACGAGAAACA
CAAAGTCTATGCCTGCGAAGTCACCCATCAGGGCCTGAGCTCGCCCGTCACAAAGAGC
TTCAACAGGGGAGAGTGTTAG
Anti-DCIR_31A6.1F5_K-var2-V-hIgGK-C (SEQ ID NO: 69):
METDTLLLWVLLLWVPGSTGDIVLTQSPASLAVSLGQRATISCRASESVDSYGISFMHWY
QQKPGQPPKLLIYRASNQESGIPARFSGSGSRTDFTLTINPVEADDVATYYCQQSNEDPLTF
GAGTKLEIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
Anti-DCIR_3C2.2D9_H-LV-hIgG4H-C (SEQ ID NO: 70):
ATGAACAGGCTTACTTCCTCATTGCTGCTGCTGATTGTCCCTGCATATGTCCTGTCCCA
GGTTACTCTGAAAGAGTCTGGCCCTGGGATATTGCAGCCCTCCCAGACCCTCAGTCTG
ACTTGTTCTTTCTCTGGGTTTTCACTGAGCACTTCTGGTATGGGTGTGAGCTGGATTCG
TCAGCCTTCAGGAAAGGGTCTGGAGTGGCTGGCACACATTTACTGGGATGATGACAAG
CGCTATAATCCATCCCTGAAGAGCCGGCTCACAATCTTTAAGGATCCCTCCAGCAACC
AGGTATTCCTCAGGATCACCAGTGTGGACACTGCAGATACTGCCACATACTACTGTGC
TCGAAACTCCCATTACTACGGTAGTACTTACGGGGGATACTTCGATGTCTGGGGCGCA
GGGACCACGGTCACCGTCTCCTCAGCCAAAACAAAGGGCCCATCCGTCTTCCCCCTGG
CGCCCTGCTCCAGGAGCACCTCCGAGAGCACAGCCGCCCTGGGCTGCCTGGTCAAGGA
CTACTTCCCCGAACCGGTGACGGTGTCGTGGAACTCAGGCGCCCTGACCAGCGGCGTG
CACACCTTCCCGGCTGTCCTACAGTCCTCAGGACTCTACTCCCTCAGCAGCGTGGTGAC
CGTGCCCTCCAGCAGCTTGGGCACGAAGACCTACACCTGCAACGTAGATCACAAGCCC
AGCAACACCAAGGTGGACAAGAGAGTTGAGTCCAAATATGGTCCCCCATGCCCACCCT
GCCCAGCACCTGAGTTCGAAGGGGGACCATCAGTCTTCCTGTTCCCCCCAAAACCCAA
GGACACTCTCATGATCTCCCGGACCCCTGAGGTCACGTGCGTGGTGGTGGACGTGAGC
CAGGAAGACCCCGAGGTCCAGTTCAACTGGTACGTGGATGGCGTGGAGGTGCATAAT
GCCAAGACAAAGCCGCGGGAGGAGCAGTTCAACAGCACGTACCGTGTGGTCAGCGTC
CTCACCGTCCTGCACCAGGACTGGCTGAACGGCAAGGAGTACAAGTGCAAGGTCTCCA
ACAAAGGCCTCCCGTCCTCCATCGAGAAAACCATCTCCAAAGCCAAAGGGCAGCCCC
GAGAGCCACAGGTGTACACCCTGCCCCCATCCCAGGAGGAGATGACCAAGAACCAGG
TCAGCCTGACCTGCCTGGTCAAAGGCTTCTACCCCAGCGACATCGCCGTGGAGTGGGA
GAGCAATGGGCAGCCGGAGAACAACTACAAGACCACGCCTCCCGTGCTGGACTCCGA
CGGCTCCTTCTTCCTCTACAGCAGGCTAACCGTGGACAAGAGCAGGTGGCAGGAGGGG
AATGTCTTCTCATGCTCCGTGATGCATGAGGCTCTGCACAACCACTACACACAGAAGA
GCCTCTCCCTGTCTCTGGGTAAAGCTAGCTGA
Anti-DCIR_3C2.2D9_H-LV-hIgG4H-C (SEQ ID NO: 71):
NRLTSSLLLLIVPAYVLSQQVTLKESGPGILQPSQTLSLTCSFSGFSLSTSGMGVSWIRQPSG
KGLEWLAHIYWDDDKRYNPSLKSRLTIFKDPSSNQVFLRITSVDTADTATYYCARNSHYY
GSTYGGYFDVWGAGTTVTVSSAKTKGPSVFPLAPCSRSTSESTAALGCLVKDYFPEPVTVS
WNSGALTSGVHTFPAVLQSSGLYSLSSVVTVPSSSLGTKTYTCNVDHKPSNTKVDKRVES
KYGPPCPPCPAPEFEGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGKAS.
Anti-DCIR_3C2.2D9_K-LV-hIgGK-C (SEQ ID NO: 72):
ATGGAGACAGACACACTCCTGCTATGGGTGCTGCTGCTCGGGGTTCCAGGTTCCACAG
GTAACATTGTGCTGACCCAGTCTCCAACTTCTTTCACTGTGTCTCTTGGGCAGAGGGCC
ACCATATCCTGCAGAGCCAGTGAAAGTGTTCATAGTTATGGCAATAGTTTTATGCACT
GGTACCAGCAGAAACCAGGGCAGCCACCCAAACTCCTCATCTATCTTGCATCCAACGT
AGAATCTGGGGTCCCTGCCAGGTTCAGTGGTAGTGGGTCCAGGACAGACTTCACCCTC
ACCATTGATCCTGTGGAGGCTGATGATGCTGCAACCTATTACTGTCAGCAAAATAGTG
AGGATCCGTGGACGTTCGGTGGAGGCACCAAGCTCGAGATCAAACGAACTGTGGCTG
CACCATCTGTCTTCATCTTCCCGCCATCTGATGAGCAGTTGAAATCTGGAACTGCCTCT
GTTGTGTGCCTGCTGAATAACTTCTATCCCAGAGAGGCCAAAGTACAGTGGAAGGTGG
ATAACGCCCTCCAATCGGGTAACTCCCAGGAGAGTGTCACAGAGCAGGACAGCAAGG
ACAGCACCTACAGCCTCAGCAGCACCCTGACGCTGAGCAAAGCAGACTACGAGAAAC
ACAAAGTCTATGCCTGCGAAGTCACCCATCAGGGCCTGAGCTCGCCCGTCACAAAGAG
CTTCAACAGGGGAGAGTGTTAG
Anti-DCIR_3C2.2D9_K-LV-hIgGK-C (SEQ ID NO: 73):
METDTLLLWVLLLGVPGSTGNIVLTQSPTSFTVSLGQRATISCRASESVHSYGNSFMHWYQ
QKPGQPPKLLIYLASNVESGVPARFSGSGSRTDFTLTIDPVEADDAATYYCQQNSEDPWTF
GGGTKLEIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
Anti-DCIR_6C8.1G9_H-V-hIgG4H-C (SEQ ID NO: 74):
ATGGAATGGACCTGGGTCTTTCTCTTCCTCCTGTCAGTAACTGCAGGTGTCCACTCCCA
GGTTCAGCTGCAGCAGTCTGGAACTGAGCTGATGAAGCCTGGGGCCTCAGTGAAGATA
TCCTGCAAGGCTACTGGCTACACATTCAGTACCTACTGGATAGAGTGGGTAAAGCAGA
GGCCTGGACATGGCCTTGAGTGGATTGGAGAGATTTTACCTGGAAGTGGTAGGACTAA
CGACAATGAGAAGTTCAAGGGCAAGGCCACAATCACTGCAGATACATCCTCCAAGAA
AGCCTACATGCAACTCAGCAGCCTGACATCTGAGGACTCTGCCGTCTATTACTGTGCA
AGAAGGGGTGGTTACTCCTTTGCTTTCTGGGGCCAAGGGACTCTGGTCTCTGTCTCTGC
AGCCAAAACAAAGGGCCCATCCGTCTTCCCCCTGGCGCCCTGCTCCAGGAGCACCTCC
GAGAGCACAGCCGCCCTGGGCTGCCTGGTCAAGGACTACTTCCCCGAACCGGTGACGG
TGTCGTGGAACTCAGGCGCCCTGACCAGCGGCGTGCACACCTTCCCGGCTGTCCTACA
GTCCTCAGGACTCTACTCCCTCAGCAGCGTGGTGACCGTGCCCTCCAGCAGCTTGGGC
ACGAAGACCTACACCTGCAACGTAGATCACAAGCCCAGCAACACCAAGGTGGACAAG
AGAGTTGAGTCCAAATATGGTCCCCCATGCCCACCCTGCCCAGCACCTGAGTTCGAAG
GGGGACCATCAGTCTTCCTGTTCCCCCCAAAACCCAAGGACACTCTCATGATCTCCCG
GACCCCTGAGGTCACGTGCGTGGTGGTGGACGTGAGCCAGGAAGACCCCGAGGTCCA
GTTCAACTGGTACGTGGATGGCGTGGAGGTGCATAATGCCAAGACAAAGCCGCGGGA
GGAGCAGTTCAACAGCACGTACCGTGTGGTCAGCGTCCTCACCGTCCTGCACCAGGAC
TGGCTGAACGGCAAGGAGTACAAGTGCAAGGTCTCCAACAAAGGCCTCCCGTCCTCCA
TCGAGAAAACCATCTCCAAAGCCAAAGGGCAGCCCCGAGAGCCACAGGTGTACACCC
TGCCCCCATCCCAGGAGGAGATGACCAAGAACCAGGTCAGCCTGACCTGCCTGGTCAA
AGGCTTCTACCCCAGCGACATCGCCGTGGAGTGGGAGAGCAATGGGCAGCCGGAGAA
CAACTACAAGACCACGCCTCCCGTGCTGGACTCCGACGGCTCCTTCTTCCTCTACAGCA
GGCTAACCGTGGACAAGAGCAGGTGGCAGGAGGGGAATGTCTTCTCATGCTCCGTGAT
GCATGAGGCTCTGCACAACCACTACACACAGAAGAGCCTCTCCCTGTCTCTGGGTAAA
GCTAGCTGA
Anti-DCIR_6C8.1G9_H-V-hIgG4H-C (SEQ ID NO: 75):
MEWTWVFLFLLSVTAGVHSQVQLQQSGTELMKPGASVKISCKATGYTFSTYWIEWVKQR
PGHGLEWIGEILPGSGRTNDNEKFKGKATITADTSSKKAYMQLSSLTSEDSAVYYCARRG
GYSFAFWGQGTLVSVSAAKTKGPSVFPLAPCSRSTSESTAALGCLVKDYFPEPVTVSWNS
GALTSGVHTFPAVLQSSGLYSLSSVVTVPSSSLGTKTYTCNVDHKPSNTKVDKRVESKYGP
PCPPCPAPEFEGGPSVFLFPPKPKDTLMISRTPEVTCVVVDVSQEDPEVQFNWYVDGVEVH
NAKTKPREEQFNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTPPVLDSDGSFFL
YSRLTVDKSRWQEGNVFSCSVMHEALHNHYTQKSLSLSLGKAS
Anti-DCIR_6C8.1G9_K-V-hIgGK-C (SEQ ID NO: 76):
ATGACCATGTTCTCACTAGCTCTTCTCCTCAGTCTTCTTCTCCTCTGTGTCTCTGATTCT
AGGGCAGAAACAACTGTGACCCAGTCTCCAGCATCCCTGTCCATGGCTATAGGAGAAA
AAGTCACCATCAGATGCGTAACCAGCACTGATATTGATGATGATGTGAACTGGTACCA
GCAGAAGCCAGGGGAACCTCCTAAGCTCCTTATTTCAGAAGGCAATACTCTTCGTGCT
GGAGTCCCATCCCGATTCTCCAGCAGTGGCTATGGTACAGATTTTGTTTTTACAATTGA
GAACATGCTCTCAGAAGATGTTGCAGATTACTACTGTTTGCAAAGTGGTAACTTGCCG
TACACGTTCGGAGGGGGGACCAAGCTCGAGATCAAACGAACTGTGGCTGCACCATCT
GTCTTCATCTTCCCGCCATCTGATGAGCAGTTGAAATCTGGAACTGCCTCTGTTGTGTG
CCTGCTGAATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGACAGCACC
TACAGCCTCAGCAGCACCCTGACGCTGAGCAAAGCAGACTACGAGAAACACAAAGTC
TATGCCTGCGAAGTCACCCATCAGGGCCTGAGCTCGCCCGTCACAAAGAGCTTCAACA
GGGGAGAGTGTTAG
Anti-DCIR_6C8.1G9_K-V-hIgGK-C (SEQ ID NO: 77):
MTMFSLALLLSLLLLCVSDSRAETTVTQSPASLSMAIGEKVTIRCVTSTDIDDDVNWYQQK
PGEPPKLLISEGNTLRAGVPSRFSSSGYGTDFVFTIENMLSEDVADYYCLQSGNLPYTFGGG
TKLEIKRTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC
Anti-DCIR_9E8.1E3_H-V-hIgG4H-C (SEQ ID NO: 78):
ATGAACAGGCTTACTTCCTCATTGCTGCTGCTGATTGTCCCTGCATATGTCCTGTCCCA
GGTTACTCTGAAAGAGTCTGGCCCTGGGATATTGCAGCCCTCCCAGACCCTCAGTCTG
ACTTGTTCTTTCTCTGGGTTTTCACTGAGCACTTCTGGTATGGGTCTGAGCTGGATTCGT
CAGCCTTCAGGAAAGGGTCTGGAGTGGCTGGCACACATTTACTGGGATGATGACAAGC
GCTATAACCCATCCCTGAAGAGCCGGCTCACAATCTCCAAGGATACCTCCAGCAACCA
GGTTTTCCTCAAGATCACCATTGTGGACACTGCAGATGCTGCCACATACTACTGTGCTC
GAAGCTCCCATTACTACGGTTATGGCTACGGGGGATACTTCGATGTCTGGGGCGCAGG
GACCACGGTCACCGTCTCCTCAGCCAAAACGAAGGGCCCATCCGTCTTCCCCCTGGCG
CCCTGCTCCAGGAGCACCTCCGAGAGCACAGCCGCCCTGGGCTGCCTGGTCAAGGACT
ACTTCCCCGAACCGGTGACGGTGTCGTGGAACTCAGGCGCCCTGACCAGCGGCGTGCA
CACCTTCCCGGCTGTCCTACAGTCCTCAGGACTCTACTCCCTCAGCAGCGTGGTGACCG
TGCCCTCCAGCAGCTTGGGCACGAAGACCTACACCTGCAACGTAGATCACAAGCCCAG
CAACACCAAGGTGGACAAGAGAGTTGAGTCCAAATATGGTCCCCCATGCCCACCCTGC
CCAGCACCTGAGTTCGAAGGGGGACCATCAGTCTTCCTGTTCCCCCCAAAACCCAAGG
ACACTCTCATGATCTCCCGGACCCCTGAGGTCACGTGCGTGGTGGTGGACGTGAGCCA
GGAAGACCCCGAGGTCCAGTTCAACTGGTACGTGGATGGCGTGGAGGTGCATAATGC
CAAGACAAAGCCGCGGGAGGAGCAGTTCAACAGCACGTACCGTGTGGTCAGCGTCCT
CACCGTCCTGCACCAGGACTGGCTGAACGGCAAGGAGTACAAGTGCAAGGTCTCCAA
CAAAGGCCTCCCGTCCTCCATCGAGAAAACCATCTCCAAAGCCAAAGGGCAGCCCCG
AGAGCCACAGGTGTACACCCTGCCCCCATCCCAGGAGGAGATGACCAAGAACCAGGT
CAGCCTGACCTGCCTGGTCAAAGGCTTCTACCCCAGCGACATCGCCGTGGAGTGGGAG
AGCAATGGGCAGCCGGAGAACAACTACAAGACCACGCCTCCCGTGCTGGACTCCGAC
GGCTCCTTCTTCCTCTACAGCAGGCTAACCGTGGACAAGAGCAGGTGGCAGGAGGGG
AATGTCTTCTCATGCTCCGTGATGCATGAGGCTCTGCACAACCACTACACACAGAAGA
GCCTCTCCCTGTCTCTGGGTAAAGCTAGCTGA
Anti-DCIR_9E8.1E3_H-V-hIgG4H-C (SEQ ID NO: 79):
MNRLTSSLLLLIVPAYVLSQVTLKESGPGILQPSQTLSLTCSFSGFSLSTSGMGLSWIRQPSG
KGLEWLAHIYWDDDKRYNPSLKSRLTISKDTSSNQVFLKITIVDTADAATYYCARSSHYY
GYGYGGYFDVWGAGTTVTVSSAKTKGPSVFPLAPCSRSTSESTAALGCLVKDYFPEPVTV
SWNSGALTSGVHTFPAVLQSSGLYSLSSVVTVPSSSLGTKTYTCNVDHKPSNTKVDKRVES
KYGPPCPPCPAPEFEGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGKAS
Anti-DCIR_9E8.1E3_K-LV-hIgGK-C (SEQ ID NO: 80):
ATGGAGACAGACACACTCCTGCTATGGGTGCTGCTGCTCTGGGTTCCAGGTTCCACAG
GTAACATTGTGCTGACCCAATCTCCAGCTTCTTTGGCTGTGTCTCTAGGGCAGAGGGCC
ACCATATCCTGCAGAGCCAGTGAAAGTATTCATAGTTATGGCAATAGTTTTCTGCACT
GGTACCAGCAGAAACCAGGACAGCCACCCAAACTCCTCATCTATCTTGCATCCAACCT
AGAATCTGGGGTCCCTGCCAGGTTCAGCGGCAGTGGGTCTAGGACAGACTTCACCCTC
ACCATTGATCCTGTGGAGGCTGATGATGCTGCAACCTATTACTGTCAGCAAAATAATG
AGGATCCGTGGACGTTCGGTGGAGGCACCAAGCTCGAGATCAAACGAACTGTGGCTG
CACCATCTGTCTTCATCTTCCCGCCATCTGATGAGCAGTTGAAATCTGGAACTGCCTCT
GTTGTGTGCCTGCTGAATAACTTCTATCCCAGAGAGGCCAAAGTACAGTGGAAGGTGG
ATAACGCCCTCCAATCGGGTAACTCCCAGGAGAGTGTCACAGAGCAGGACAGCAAGG
ACAGCACCTACAGCCTCAGCAGCACCCTGACGCTGAGCAAAGCAGACTACGAGAAAC
ACAAAGTCTATGCCTGCGAAGTCACCCATCAGGGCCTGAGCTCGCCCGTCACAAAGAG
CTTCAACAGGGGAGAGTGTTAGGCGGCCGCACTAGCGCGGGCCGCATTCGAAGAGCT
CGGTACCCGGGGATCCTCTAGAGTCGACCTGCAGGCATGCAAGCTGGCCGCGACTCTA
GATCATAATCAGC
Anti-DCIR_9E8.1E3_K-LV-hIgGK-C (SEQ ID NO: 81):
METDTLLLWVLLLWVPGSTGNIVLTQSPASLAVSLGQRATISCRASESIHSYGNSFLHWYQ
QKPGQPPKLLIYLASNLESGVPARFSGSGSRTDFTLTIDPVEADDAATYYCQQNNEDPWTF
GGGTKLEIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
Anti-DCIR2C9H-LV-hIgG4H-V-hIgG4H-C (SEQ ID NO: 82):
ATGAAATGCAGCTGGGTCATCTTCTTCCTGATGGCAGTGGTTACAGGGGTCAATTCAG
AGGTTCAGCTGCAGCAGTCTGGGGCTGAGCTTGTGAGGCCAGGGGCCTTAGTCAAGTT
GTCCTGCAAAGCTTCTGGCTTCAACATTAATGACTACTATATCCACTGGGTGAAGCAG
CGGCCTGAACAGGGCCTGGAGCGGATTGGATGGATTGATCCTGACAATGGTAATACTA
TATATGACCCGAAGTTCCAGGGCAAGGCCAGTATAACAGCAGACACATCCCCCAACA
CAGCCTACCTGCAGCTCAGCAGCCTGACATCTGAGGACACTGCCGTCTATTACTGTGC
TAGAACCCGATCTCCTATGGTTACGACGGGGTTTGTTTACTGGGGCCAAGGGACTGTG
GTCACTGTCTCTGCAGCCAAAACGAAGGGCCCATCCGTCTTCCCCCTGGCGCCCTGCTC
CAGGAGCACCTCCGAGAGCACAGCCGCCCTGGGCTGCCTGGTCAAGGACTACTTCCCC
GAACCGGTGACGGTGTCGTGGAACTCAGGCGCCCTGACCAGCGGCGTGCACACCTTCC
CGGCTGTCCTACAGTCCTCAGGACTCTACTCCCTCAGCAGCGTGGTGACCGTGCCCTCC
AGCAGCTTGGGCACGAAGACCTACACCTGCAACGTAGATCACAAGCCCAGCAACACC
AAGGTGGACAAGAGAGTTGAGTCCAAATATGGTCCCCCATGCCCACCCTGCCCAGCAC
CTGAGTTCGAAGGGGGACCATCAGTCTTCCTGTTCCCCCCAAAACCCAAGGACACTCT
CATGATCTCCCGGACCCCTGAGGTCACGTGCGTGGTGGTGGACGTGAGCCAGGAAGAC
CCCGAGGTCCAGTTCAACTGGTACGTGGATGGCGTGGAGGTGCATAATGCCAAGACRA
AGCCGCGGGAGGAGCAGTTCAACAGCACGTACCGTGTGGTCAGCGTCCTCACCGTCCT
GCACCAGGACTGGCTGAACGGCAAGGAGTACAAGTGCAAGGTCTCCAACAAAGGCCT
CCCGTCCTCCATCGAGAAAACCATCTCCAAAGCCAAAGGGCAGCCCCGAGAGCCACA
GGTGTACACCCTGCCCCCATCCCAGGAGGAGATGACCAAGAACCAGGTCAGCCTGAC
CTGCCTGGTCAAAGGCTTCTACCCCAGCGACATCGCCGTGGAGTGGGAGAGCAATGGG
CAGCCGGAGAACAACTACAAGACCACGCCTCCCGTGCTGGACTCCGACGGCTCCTTCT
TCCTCTACAGCAGGCTAACCGTGGACAAGAGCAGGTGGCAGGAGGGGAATGTCTTCTC
ATGCTCCGTGATGCATGAGGCTCTGCACAACCACTACACACAGAAGAGCCTCTCCCTG
TCTCTGGGTAAATGA
Anti-DCIR2C9H-LV-hIgG4H-V-hIgG4H-C (SEQ ID NO: 83):
MKCSWVIFFLMAVVTGVNSEVQLQQSGAELVRPGALVKLSCKASGFNINDYYIHWVKQR
PEQGLERIGWIDPDNGNTIYDPKFQGKASITADTSPNTAYLQLSSLTSEDTAVYYCARTRSP
MVTTGFVYWGQGTVVTVSAAKTKGPSVFPLAPCSRSTSESTAALGCLVKDYFPEPVTVSW
NSGALTSGVHTFPAVLQSSGLYSLSSVVTVPSSSLGTKTYTCNVDHKPSNTKVDKRVESKY
GPPCPPCPAPEFEGGPSVFLFPPKPKDTLMISRTPEVTCVVVDVSQEDPEVQFNWYVDGVE
VHNAKXKPREEQFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYKTTPPVLDSDGSF
FLYSRLTVDKSRWQEGNVFSCSVMHEALHNHYTQKSLSLSLGK
Anti-DC-SIGNL16E3H (SEQ ID NO: 84):
ATGGAAAGGCACTGGATCTTTCTCTTCCTGTTTTCAGTAACTGCAGGTGTCCACTCCCA
GGTCCAGCTTCAGCAGTCTGGGGCTGAGCTGGCAAAACCTGGGGCCTCAGTGAAGATG
TCCTGCAAGGCTTCTGGCTACACCTTTACTACCTACTGGATGCACTGGGTAAAACAGA
GGCCTGGACAGGGTCTGGAATGGATTGGATACATTAATCCTATCACTGGTTATACTGA
GTACAATCAGAAGTTCAAGGACAAGGCCACCTTGACTGCAGACAAATCCTCCAGCAC
AGCCTACATGCAACTGAGCAGCCTGACATCTGAGGACTCTGCAGTCTATTACTGTGCA
AGAGAGGGTTTAAGTGCTATGGACTATTGGGGTCAGGGAACCTCAGTCACCGTCACCT
CAGCCAAAACAACAGCCCCATCGGTCTATCCACTGGCCCCTGTGTGTGGAGATACAAC
TGGCTCCTCGGTAACTCTAGGATGCCTGGTCAAGGGTTATTTCCCTGAGCCAGTGACCT
TGACCTGGAACTCTGGATCCCTGTCCAGTGGTGTGCACACCTTCCCAGCTGTCCTGCAG
TCTGACCTCTACACCCTCAGCAGCTCAGTGACTGTAACCTCGAGCACCTGGCCCAGCC
AGACCGTCACCTGCAGCGTTGCTCACCCAGCCAGCAGCACCACGGTGGACAAAAAAC
TTGAGCCCAGCGGGCCCATTTCAACAATCAACCCCTGTCCTCCATGCAAGGAGTGTCA
CAAATGCCCAGCTCCTAACCTCGAGGGTGGACCATCCGTCTTCATCTTCCCTCCAAATA
TCAAGGATGTACTCATGATCTCCCTGACACCCAAGGTCACGTGTGTGGTGGTGGATGT
GAGCGAGGATGACCCAGACGTCCAGATCAGCTGGTTTGTGAACAACGTGGAAGTACA
CACAGCTCAGACACAAACCCATAGAGAGGATTACAACAGTACTATCCGGGTGGTCAG
CACCCTCCCCATCCAGCACCAGGACTGGATGAGTGGCAAGGAGTTCAAATGCAAGGTC
AACAACAAAGACCTCCCATCACCCATCGAGAGAACCATCTCAAAAATTAAAGGGCTA
GTCAGAGCTCCACAAGTATACATCTTGCCGCCACCAGCAGAGCAGTTGTCCAGGAAAG
ATGTCAGTCTCACTTGCCTGGTCGTGGGCTTCAACCCTGGAGACATCAGTGTGGAGTG
GACCAGCAATGGGCATACAGAGGAGAACTACAAGGACACCGCACCAGTCCTGGACTC
TGACGGTTCTTACTTCATATATAGCAAGCTCAATATGAAAACAAGCAAGTGGGAGAAA
ACAGATTCCTTCTCATGCAACGTGAGACACGAGGGTCTGAAAAATTACTACCTGAAGA
AGACCATCTCCCGGTCTCCGGGTAAAGCTAGCTGA
Anti-DC-SIGNL16E3H (SEQ ID NO: 85):
MERHWIFLFLFSVTAGVHSQVQLQQSGAELAKPGASVKMSCKASGYTFTTYWMHWVKQ
RPGQGLEWIGYINPITGYTEYNQKFKDKATLTADKSSSTAYMQLSSLTSEDSAVYYCAREG
LSAMDYWGQGTSVTVTSAKTTAPSVYPLAPVCGDTTGSSVTLGCLVKGYFPEPVTLTWNS
GSLSSGVHTFPAVLQSDLYTLSSSVTVTSSTWPSQTVTCSVAHPASSTTVDKKLEPSGPISTI
NPCPPCKECHKCPAPNLEGGPSVFIFPPNIKDVLMISLTPKVTCVVVDVSEDDPDVQISWFV
NNVEVHTAQTQTHREDYNSTIRVVSTLPIQHQDWMSGKEFKCKVNNKDLPSPIERTISKIK
GLVRAPQVYILPPPAEQLSRKDVSLTCLVVGFNPGDISVEWTSNGHTEENYKDTAPVLDSD
GSYFIYSKLNMKTSKWEKTDSFSCNVRHEGLKNYYLKKTISRSPGKAS.
Anti-DC-SIGNL16E3K (SEQ ID NO: 86):
ATGGGCATCAAGATGGAGTCACGGATTCAGGCATTTGTATTCGTGTTTCTCTGGTTGTC
TGGTGTTGGCGGAGACATTGTGATGACCCAGTCTCACAAATTCATGTCCACATCAGTA
GGAGACAGGGTCAGCGTCACCTGCAAGGCCAGTCAGGATGTGACTTCTGCTGTAGCCT
GGTATCAACAAAAACCAGGGCAATCTCCTAAACTACTGATTTACTGGGCATCCACCCG
GCACACTGGAGTCCCTGATCGCTTCACAGGCAGTGGATCTGGGACAGATTATACTCTC
ACCATCAGCAGTGGGCAGGCTGAAGACCTGGCACTTTATTACTGTCACCAATATTATA
GCGCTCCTCGGACGTTCGGTGGAGGCACCAAGCTGGAAGTCAAACGGGCTGATGCTGC
ACCAACTGTATCCATCTTCCCACCATCCAGTGAGCAGTTAACATCTGGAGGTGCCTCA
GTCGTGTGCTTCTTGAACAACTTCTACCCCAAAGACATCAATGTCAAGTGGAAGATTG
ATGGCAGTGAACGACAAAATGGCGTCCTGAACAGTTGGACTGATCAGGACAGCAAAG
ACAGCACCTACAGCATGAGCAGCACCCTCACGTTGACCAAGGACGAGTATGAACGAC
ATAACAGCTATACCTGTGAGGCCACTCACAAGACATCAACTTCACCCATCGTCAAGAG
CTTCAATAGGAATGAGTGTTAG
Anti-DC-SIGNL16E3K (SEQ ID NO: 87):
MESRIQAFVFVFLWLSGVGGDIVMTQSHKFMSTSVGDRVSVTCKASQDVTSAVAWYQQK
PGQSPKLLIYWASTRHTGVPDRFTGSGSGTDYTLTISSGQAEDLALYYCHQYYSAPRTFGG
GTKLEVKRADAAPTVSIFPPSSEQLTSGGASVVCFLNNFYPKDINVKWKIDGSERQNGVLN
SWTDQDSKDSTYSMSSTLTLTKDEYERHNSYTCEATHKTSTSPIVKSFNRNEC
Anti-DC-SIGNL16E7H-LV-hIgG4H-C (SEQ ID NO: 88):
ATGGAAAGGCACTGGATCTTTCTCTTCCTGTTTTCAGTAACTGCAGGTGTCCACTCCCA
GGTCCAGCTTCAGCAGTCTGGGGCTGAGCTGGCAAAACCTGGGGCCTCAGTGAAGATG
TCCTGCAAGGCTTCTGGCTACACCTTTACTACCTACTGGATGCACTGGGTAAAACAGA
GGCCTGGACAGGGTCTGGAATGGATTGGATACATTAATCCTATCACTGGTTATACTGA
GTACAATCAGAAGTTCAAGGACAAGGCCACCTTGACTGCAGACAAATCCTCCAGCAC
AGCCTACATGCAACTGAGCAGCCTGACATCTGAGGACTCTGCAGTCTATTACTGTGCA
AGAGAGGGTTTAAGTGCTATGGACTATTGGGGTCAGGGAACCTCAGTCACCGTCACCT
CAGCCAAAACAACGGGCCCATCCGTCTTCCCCCTGGCGCCCTGCTCCAGGAGCACCTC
CGAGAGCACAGCCGCCCTGGGCTGCCTGGTCAAGGACTACTTCCCCGAACCGGTGACG
GTGTCGTGGAACTCAGGCGCCCTGACCAGCGGCGTGCACACCTTCCCGGCTGTCCTAC
AGTCCTCAGGACTCTACTCCCTCAGCAGCGTGGTGACCGTGCCCTCCAGCAGCTTGGG
CACGAAGACCTACACCTGCAACGTAGATCACAAGCCCAGCAACACCAAGGTGGACAA
GAGAGTTGAGTCCAAATATGGTCCCCCATGCCCACCCTGCCCAGCACCTGAGTTCGAA
GGGGGACCATCAGTCTTCCTGTTCCCCCCAAAACCCAAGGACACTCTCATGATCTCCC
GGACCCCTGAGGTCACGTGCGTGGTGGTGGACGTGAGCCAGGAAGACCCCGAGGTCC
AGTTCAACTGGTACGTGGATGGCGTGGAGGTGCATAATGCCAAGACAAAGCCGCGGG
AGGAGCAGTTCAACAGCACGTACCGTGTGGTCAGCGTCCTCACCGTCCTGCACCAGGA
CTGGCTGAACGGCAAGGAGTACAAGTGCAAGGTCTCCAACAAAGGCCTCCCGTCCTCC
ATCGAGAAAACCATCTCCAAAGCCAAAGGGCAGCCCCGAGAGCCACAGGTGTACACC
CTGCCCCCATCCCAGGAGGAGATGACCAAGAACCAGGTCAGCCTGACCTGCCTGGTCA
AAGGCTTCTACCCCAGCGACATCGCCGTGGAGTGGGAGAGCAATGGGCAGCCGGAGA
ACAACTACAAGACCACGCCTCCCGTGCTGGACTCCGACGGCTCCTTCTTCCTCTACAGC
AGGCTAACCGTGGACAAGAGCAGGTGGCAGGAGGGGAATGTCTTCTCATGCTCCGTG
ATGCATGAGGCTCTGCACAACCACTACACACAGAAGAGCCTCTCCCTGTCTCTGGGTA
AAGCTAGCTGA
Anti-DC-SIGNL16E7H-LV-hIgG4H-C (SEQ ID NO: 89):
MERHWIFLFLFSVTAGVHSQVQLQQSGAELAKPGASVKMSCKASGYTFTTYWMHWVKQ
RPGQGLEWIGYINPITGYTEYNQKFKDKATLTADKSSSTAYMQLSSLTSEDSAVYYCAREG
LSAMDYWGQGTSVTVTSAKTTGPSVFPLAPCSRSTSESTAALGCLVKDYFPEPVTVSWNS
GALTSGVHTFPAVLQSSGLYSLSSVVTVPSSSLGTKTYTCNVDHKPSNTKVDKRVESKYGP
PCPPCPAPEFEGGPSVFLFPPKPKDTLMISRTPEVTCVVVDVSQEDPEVQFNWYVDGVEVH
NAKTKPREEQFNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTPPVLDSDGSFFL
YSRLTVDKSRWQEGNVFSCSVMHEALHNHYTQKSLSLSLGKAS.
Anti-DC-SIGNL16E7K-LV-hIgGK-C (SEQ ID NO: 90):
ATGGGCATCAAGATGGAGTCACAGATTCAGGCATTTGTATTCGTGTTTCTCTGGTTGTC
TGGTGTTGGCGGAGACATTGTGATGACCCAGTCTCACAAATTCATGTCCACATCAGTA
GGAGACAGGGTCAGCGTCACCTGCAAGGCCAGTCAGGATGTGACTTCTGCTGTAGCCT
GGTATCAACAAAAACCAGGGCAATCTCCTAAACTACTGATTTACTGGGCATCCACCCG
GCACACTGGAGTCCCTGATCGCTTCACAGGCAGTGGATCTGGGACAGATTATACTCTC
ACCATCAGCAGTGGGCAGGCTGAAGACCTGGCACTTTATTACTGTCACCAATATTATA
GCGCTCCTCGGACGTTCGGTGGAGGCACCAAGCTCGAGATCAAACGAACTGTGGCTGC
ACCATCTGTCTTCATCTTCCCGCCATCTGATGAGCAGTTGAAATCTGGAACTGCCTCTG
TTGTGTGCCTGCTGAATAACTTCTATCCCAGAGAGGCCAAAGTACAGTGGAAGGTGGA
TAACGCCCTCCAATCGGGTAACTCCCAGGAGAGTGTCACAGAGCAGGACAGCAAGGA
CAGCACCTACAGCCTCAGCAGCACCCTGACGCTGAGCAAAGCAGACTACGAGAAACA
CAAAGTCTATGCCTGCGAAGTCACCCATCAGGGCCTGAGCTCGCCCGTCACAAAGAGC
TTCAACAGGGGAGAGTGTTAG
Anti-DC-SIGNL16E7K-LV-hIgGK-C (SEQ ID NO: 91):
MESQIQAFVFVFLWLSGVGGDIVMTQSHKFMSTSVGDRVSVTCKASQDVTSAVAWYQQK
PGQSPKLLIYWASTRHTGVPDRFTGSGSGTDYTLTISSGQAEDLALYYCHQYYSAPRTFGG
GTKLEIKRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC
Anti-Dectin_1_11B6.4_H-V-hIgG4H-C (SEQ ID NO: 92):
ATGGCTGTCCTGGCACTACTCCTCTGCCTGGTGGCTTTCCCAACTTGTACCCTGTCCCA
GGTGCAACTGAAGGAGTCAGGACCTGGCCTGGTGGCGCCCTCACAGAGCCTGTCCATT
ACCTGCTCTGTCTCTGGGTTCTCATTAAGCAACTATGATATAAGCTGGATTCGCCAGCC
ACCAGGAAAGGGTCTGGAGTGGCTTGGAGTAATGTGGACTGGTGGAGGCGCAAATTA
TAATTCAGCTTTCATGTCCAGACTGAGCATCAACAAGGACAACTCCAAGAGCCAAGTT
TTTTTAAAAATGAACAATCTGCAAACTGATGACACAGCCATTTATTACTGTGTCAGAG
ATGCGGTGAGGTACTGGAACTTCGATGTCTGGGGCGCAGGGACCACGGTCACCGTCTC
CTCAGCCAAAACGAAGGGCCCATCCGTCTTCCCCCTGGCGCCCTGCTCCAGGAGCACC
TCCGAGAGCACAGCCGCCCTGGGCTGCCTGGTCAAGGACTACTTCCCCGAACCGGTGA
CGGTGTCGTGGAACTCAGGCGCCCTGACCAGCGGCGTGCACACCTTCCCGGCTGTCCT
ACAGTCCTCAGGACTCTACTCCCTCAGCAGCGTGGTGACCGTGCCCTCCAGCAGCTTG
GGCACGAAGACCTACACCTGCAACGTAGATCACAAGCCCAGCAACACCAAGGTGGAC
AAGAGAGTTGAGTCCAAATATGGTCCCCCATGCCCACCCTGCCCAGCACCTGAGTTCG
AAGGGGGACCATCAGTCTTCCTGTTCCCCCCAAAACCCAAGGACACTCTCATGATCTC
CCGGACCCCTGAGGTCACGTGCGTGGTGGTGGACGTGAGCCAGGAAGACCCCGAGGT
CCAGTTCAACTGGTACGTGGATGGCGTGGAGGTGCATAATGCCAAGACAAAGCCGCG
GGAGGAGCAGTTCAACAGCACGTACCGTGTGGTCAGCGTCCTCACCGTCCTGCACCAG
GACTGGCTGAACGGCAAGGAGTACAAGTGCAAGGTCTCCAACAAAGGCCTCCCGTCC
TCCATCGAGAAAACCATCTCCAAAGCCAAAGGGCAGCCCCGAGAGCCACAGGTGTAC
ACCCTGCCCCCATCCCAGGAGGAGATGACCAAGAACCAGGTCAGCCTGACCTGCCTGG
TCAAAGGCTTCTACCCCAGCGACATCGCCGTGGAGTGGGAGAGCAATGGGCAGCCGG
AGAACAACTACAAGACCACGCCTCCCGTGCTGGACTCCGACGGCTCCTTCTTCCTCTA
CAGCAGGCTAACCGTGGACAAGAGCAGGTGGCAGGAGGGGAATGTCTTCTCATGCTC
CGTGATGCATGAGGCTCTGCACAACCACTACACACAGAAGAGCCTCTCCCTGTCTCTG
GGTAAAGCTAGCTGA
Anti-Dectin_1_11B6.4_H-V-hIgG4H-C (SEQ ID NO: 93):
MAVLALLLCLVAFPTCTLSQVQLKESGPGLVAPSQSLSITCSVSGFSLSNYDISWIRQPPGK
GLEWLGVMWTGGGANYNSAFMSRLSINKDNSKSQVFLKMNNLQTDDTAIYYCVRDAVR
YWNFDVWGAGTTVTVSSAKTKGPSVFPLAPCSRSTSESTAALGCLVKDYFPEPVTVSWNS
GALTSGVHTFPAVLQSSGLYSLSSVVTVPSSSLGTKTYTCNVDHKPSNTKVDKRVESKYGP
PCPPCPAPEFEGGPSVFLFPPKPKDTLMISRTPEVTCVVVDVSQEDPEVQFNWYVDGVEVH
NAKTKPREEQFNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTPPVLDSDGSFFL
YSRLTVDKSRWQEGNVFSCSVMHEALHNHYTQKSLSLSLGKAS
Anti-Dectin_1_11B6.4_K-LV-hIgGK-C (SEQ ID NO: 94):
ATGGATTTTCAAGCGCAGATTTTCAGCTTCCTGCTAATCAGTGCTTCAGTCATAATGTC
CAGAGGACAAATTGTTCTCTCCCAGTCACCAGCAATCCTGTCTGCATCTCCAGGGGAG
AAGGTCACAATGACTTGCAGGGCCAGCTCAAGTGTAAGTTACATACACTGGTACCAGC
AGAAGCCAGGATCCTCCCCCAAACCCTGGATTTATGCCACATCCCACCTGGCTTCTGG
AGTCCCTGCTCGCTTCAGTGGCAGTGGGTCTGGGACCTCTTACTCTCTCACAATCAGCA
GAGTGGAGGCTGAAGATACTGCCACTTATTACTGCCAGCAGTGGAGTAGTAACCCATT
CACGTTCGGCTCGGGGACAAAGCTCGAGATCAAACGAACTGTGGCTGCACCATCTGTC
TTCATCTTCCCGCCATCTGATGAGCAGTTGAAATCTGGAACTGCCTCTGTTGTGTGCCT
GCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTAC
AGCCTCAGCAGCACCCTGACGCTGAGCAAAGCAGACTACGAGAAACACAAAGTCTAT
GCCTGCGAAGTCACCCATCAGGGCCTGAGCTCGCCCGTCACAAAGAGCTTCAACAGGG
GAGAGTGTTAG
Anti-Dectin_1_11B6.4_K-LV-hIgGK-C (SEQ ID NO: 95):
MDFQAQIFSFLLISASVIMSRGQIVLSQSPAILSASPGEKVTMTCRASSSVSYIHWYQQKPGS
SPKPWIYATSHLASGVPARFSGSGSGTSYSLTISRVEAEDTATYYCQQWSSNPFTFGSGTKL
EIKRTVAAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
Anti-Dectin_1_15E2.5_H-V-hIgG4H-C (SEQ ID NO: 96):
ATGGAAAGGCACTGGATCTTTCTACTCCTGTTGTCAGTAACTGCAGGTGTCCACTCCCA
GGTCCAGCTGCAGCAGTCTGGGGCTGAACTGGCAAGACCTGGGGCCTCAGTGAAGAT
GTCCTGCAAGGCTTCTGGCTACACCTTTACTACCTACACTATGCACTGGGTAAAACAG
AGGCCTGGACAGGGTCTGGAATGGATTGGATACATTAATCCTAGCAGTGGTTATACTA
ATTACAATCAGAAGTTCAAGGACAAGGCCACATTGACTGCAGACAAATCCTCCAGCAC
AGCCTCCATGCAACTGAGCAGCCTGACATCTGAGGACTCTGCAGTCTATTACTGTGCA
AGAGAGAGGGCGGTATTAGTCCCCTATGCTATGGACTACTGGGGTCAAGGAACCTCAG
TCACCGTCTCCTCAGCCAAAACAAAGGGCCCATCCGTCTTCCCCCTGGCGCCCTGCTCC
AGGAGCACCTCCGAGAGCACAGCCGCCCTGGGCTGCCTGGTCAAGGACTACTTCCCCG
AACCGGTGACGGTGTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCCCTCCA
GCAGCTTGGGCACGAAGACCTACACCTGCAACGTAGATCACAAGCCCAGCAACACCA
AGGTGGACAAGAGAGTTGAGTCCAAATATGGTCCCCCATGCCCACCCTGCCCAGCACC
TGAGTTCGAAGGGGGACCATCAGTCTTCCTGTTCCCCCCAAAACCCAAGGACACTCTC
ATGATCTCCCGGACCCCTGAGGTCACGTGCGTGGTGGTGGACGTGAGCCAGGAAGACC
CCGAGGTCCAGTTCAACTGGTACGTGGATGGCGTGGAGGTGCATAATGCCAAGACAA
AGCCGCGGGAGGAGCAGTTCAACAGCACGTACCGTGTGGTCAGCGTCCTCACCGTCCT
GCACCAGGACTGGCTGAACGGCAAGGAGTACAAGTGCAAGGTCTCCAACAAAGGCCT
CCCGTCCTCCATCGAGAAAACCATCTCCAAAGCCAAAGGGCAGCCCCGAGAGCCACA
GGTGTACACCCTGCCCCCATCCCAGGAGGAGATGACCAAGAACCAGGTCAGCCTGAC
CTGCCTGGTCAAAGGCTTCTACCCCAGCGACATCGCCGTGGAGTGGGAGAGCAATGGG
CAGCCGGAGAACAACTACAAGACCACGCCTCCCGTGCTGGACTCCGACGGCTCCTTCT
TCCTCTACAGCAGGCTAACCGTGGACAAGAGCAGGTGGCAGGAGGGGAATGTCTTCTC
ATGCTCCGTGATGCATGAGGCTCTGCACAACCACTACACACAGAAGAGCCTCTCCCTG
TCTCTGGGTAAAGCTAGCTGA
Anti-Dectin_1_15E2.5_H-V-hIgG4H-C (SEQ ID NO: 97):
MERHWIFLLLLSVTAGVHSQVQLQQSGAELARPGASVKMSCKASGYTFTTYTMHWVKQ
RPGQGLEWIGYINPSSGYTNYNQKFKDKATLTADKSSSTASMQLSSLTSEDSAVYYCARE
RAVLVPYAMDYWGQGTSVTVSSAKTKGPSVFPLAPCSRSTSESTAALGCLVKDYFPEPVT
VSWNSGALTSGVHTFPAVLQSSGLYSLSSVVTVPSSSLGTKTYTCNVDHKPSNTKVDKRV
ESKYGPPCPPCPAPEFEGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIEKTISKA
KGQPREPQVYTLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGKAS
Anti-Dectin_1_15E2.5_K-V-hIgGK-C (SEQ ID NO: 98):
ATGCATTTTCAAGTGCAGATTTTCAGCTTCCTGCTAATCAGTGCCTCAGTCATAATGTC
CAGAGGACAAATTGTTCTCACCCAGTCTCCAGCAGTCATGTCTGCATCTCCAGGGGAG
AAGGTCACCATAACCTGCACTGCCAGCTCAAGTTTAAGTTACATGCACTGGTTCCAGC
AGAAGCCAGGCACTTCTCCCAAACTCTGGCTTTATAGCACATCCATCCTGGCTTCTGGA
GTCCCTACTCGCTTCAGTGGCAGTGGATCTGGGACCTCTTACTCTCTCACAATCAGCCG
AATGGAGGCTGAAGATGCTGCCACTTATTACTGCCAGCAAAGGAGTAGTTCCCCATTC
ACGTTCGGCTCGGGGACAAAGCTCGAGATCAAACGAACTGTGGCTGCACCATCTGTCT
TCATCTTCCCGCCATCTGATGAGCAGTTGAAATCTGGAACTGCCTCTGTTGTGTGCCTG
CTGAATAACTTCTATCCCAGAGAGGCCAAAGTACAGTGGAAGGTGGATAACGCCCTCC
AATCGGGTAACTCCCAGGAGAGTGTCACAGAGCAGGACAGCAAGGACAGCACCTACA
GCCTCAGCAGCACCCTGACGCTGAGCAAAGCAGACTACGAGAAACACAAAGTCTATG
CCTGCGAAGTCACCCATCAGGGCCTGAGCTCGCCCGTCACAAAGAGCTTCAACAGGGG
AGAGTGTTAG
Anti-Dectin_1_15E2.5_K-V-hIgGK-C (SEQ ID NO: 99):
MHFQVQIFSFLLISASVIMSRGQIVLTQSPAVMSASPGEKVTITCTASSSLSYMHWFQQKPG
TSPKLWLYSTSILASGVPTRFSGSGSGTSYSLTISRMEAEDAATYYCQQRSSSPFTFGSGTKL
EIKRTVAAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
Anti-Dectin_1_2D8.2D4H-V-hIgG4H-C (SEQ ID NO: 100):
ATGGGATGGACCTGGATCTTTATTTTAATCCTGTCAGTTACTACAGGTGTCCACTCTGA
GGTCCAGCTGCAGCAGTCTGGACCTGAGCTGGAGAAGCCTGGCGCTTCAGTGAAGATA
TCCTGCAAGGCTTCTGGTTACTCCTTCACTGGCTACAACATGAACTGGGTGAAACAGA
GCAATGGAAAGAGCCTTGAGTGGATTGGAAATATTGATCCTTACTATGGTGATACTAA
CTACAACCAGAAGTTCAAGGGCAAGGCCACATTGACTGTAGACAAATCCTCCAGCAC
AGCCTACATGCACCTCAAGAGCCTGACATCTGAGGACTCTGCAGTCTATTACTGTGCA
AGACCCTACGGTAGTGAGGCCTACTTTGCTTACTGGGGCCAAGGGACTCTGGTCACTG
TCTCTGCAGCCAAAACGAAGGGCCCATCCGTCTTCCCCCTGGCGCCCTGCTCCAGGAG
CACCTCCGAGAGCACAGCCGCCCTGGGCTGCCTGGTCAAGGACTACTTCCCCGAACCG
GTGACGGTGTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCAGCAG
CTTGGGCACGAAGACCTACACCTGCAACGTAGATCACAAGCCCAGCAACACCAAGGT
GGACAAGAGAGTTGAGTCCAAATATGGTCCCCCATGCCCACCCTGCCCAGCACCTGAG
TTCGAAGGGGGACCATCAGTCTTCCTGTTCCCCCCAAAACCCAAGGACACTCTCATGA
TCTCCCGGACCCCTGAGGTCACGTGCGTGGTGGTGGACGTGAGCCAGGAAGACCCCGA
GGTCCAGTTCAACTGGTACGTGGATGGCGTGGAGGTGCATAATGCCAAGACAAAGCC
GCGGGAGGAGCAGTTCAACAGCACGTACCGTGTGGTCAGCGTCCTCACCGTCCTGCAC
CAGGACTGGCTGAACGGCAAGGAGTACAAGTGCAAGGTCTCCAACAAAGGCCTCCCG
TCCTCCATCGAGAAAACCATCTCCAAAGCCAAAGGGCAGCCCCGAGAGCCACAGGTG
TACACCCTGCCCCCATCCCAGGAGGAGATGACCAAGAACCAGGTCAGCCTGACCTGCC
TGGTCAAAGGCTTCTACCCCAGCGACATCGCCGTGGAGTGGGAGAGCAATGGGCAGC
CGGAGAACAACTACAAGACCACGCCTCCCGTGCTGGACTCCGACGGCTCCTTCTTCCT
CTACAGCAGGCTAACCGTGGACAAGAGCAGGTGGCAGGAGGGGAATGTCTTCTCATG
CTCCGTGATGCATGAGGCTCTGCACAACCACTACACACAGAAGAGCCTCTCCCTGTCT
CTGGGTAAAGCTAGCTGA
Anti-Dectin_1_2D8.2D4H-V-hIgG4H-C (SEQ ID NO: 101):
MGWTWIFILILSVTTGVHSEVQLQQSGPELEKPGASVKISCKASGYSFTGYNMNWVKQSN
GKSLEWIGNIDPYYGDTNYNQKFKGKATLTVDKSSSTAYMHLKSLTSEDSAVYYCARPY
GSEAYFAYWGQGTLVTVSAAKTKGPSVFPLAPCSRSTSESTAALGCLVKDYFPEPVTVSW
NSGALTSGVHTFPAVLQSSGLYSLSSVVTVPSSSLGTKTYTCNVDHKPSNTKVDKRVESKY
GPPCPPCPAPEFEGGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYKTTPPVLDSDGSF
FLYSRLTVDKSRWQEGNVFSCSVMHEALHNHYTQKSLSLSLGKAS
Anti-Dectin_1_2D8.2D4K-V-hIgGK-C (SEQ ID NO: 102):
ATGGTGTCCACTTCTCAGCTCCTTGGACTTTTGCTTTTCTGGACTTCAGCCTCCAGATGT
GACATTGTGATGACTCAGTCTCCAGCCACCCTGTCTGTGACTCCAGGAGATAGAGTCT
CTCTTTCCTGCAGGGCCAGCCAGAGTATTAGCGACTACTTACACTGGTATCAACAAAA
ATCACATGAGTCTCCAAGGCTTCTCATCAAATATGCTGCCCAATCCATCTCTGGGATCC
CCTCCAGGTTCAGTGGCAGTGGATCAGGGTCAGATTTCACTCTCAGTATCAACGGTGT
GGAACCTGAAGATGTTGGAGTGTATTACTGTCAAAATGGTCACAGCTTTCCGTACACG
TTCGGAGGGGGGACCAAGCTCGAGATCAAACGAACTGTGGCTGCACCATCTGTCTTCA
TCTTCCCGCCATCTGATGAGCAGTTGAAATCTGGAACTGCCTCTGTTGTGTGCCTGCTG
AATAACTTCTATCCCAGAGAGGCCAAAGTACAGTGGAAGGTGGATAACGCCCTCCAAT
CGGGTAACTCCCAGGAGAGTGTCACAGAGCAGGACAGCAAGGACAGCACCTACAGCC
TCAGCAGCACCCTGACGCTGAGCAAAGCAGACTACGAGAAACACAAAGTCTATGCCT
GCGAAGTCACCCATCAGGGCCTGAGCTCGCCCGTCACAAAGAGCTTCAACAGGGGAG
AGTGTTAG
Anti-Dectin_1_2D8.2D4K-V-hIgGK-C (SEQ ID NO: 103):
DIVMTQSPATLSVTPGDRVSLSCRASQSISDYLHWYQQKSHESPRLLIKYAAQSISGIPSRFS
GSGSGSDFTLSINGVEPEDVGVYYCQNGHSFPYTFGGGTKLEIKRTVAAPSVFIFPPSDEQL
KSGTASVVCLLNNFYPREAKVQWKVDNALQSGNSQESVTEQDSKDSTYSLSSTLTLSKAD
YEKHKVYACEVTHQGLSSPVTKSFNRGEC
Anti-Langerin15B10H-LV-hIgG4H-C (SEQ ID NO: 104):
ATGGAATGGAGGATCTTTCTCTTCATCCTGTCAGGAACTGCAGGTGTCCACTCCCAGGT
TCAGCTGCGGCAGTCTGGACCTGAGCTGGTGAAGCCTGGGGCTTCAGTGAAGATGTCC
TGCAAGGCTTCTGGATACACATTTACTGACTATGTTATAAGTTGGGTGAAGCAGAGAA
CTGGACAGGGCCTTGAGTGGATTGGAGATATTTATCCTGGAAGTGGTTATTCTTTCTAC
AATGAGAACTTCAAGGGCAAGGCCACACTGACTGCAGACAAATCCTCCACCACAGCC
TACATGCAGCTCAGCAGCCTGACATCTGAGGACTCTGCGGTCTATTTCTGTGCAACCTA
CTATAACTACCCTTTTGCTTACTGGGGCCAAGGGACTCTGGTCACTGTCTCTGCAGCCA
AAACAACGGGCCCATCCGTCTTCCCCCTGGCGCCCTGCTCCAGGAGCACCTCCGAGAG
CACAGCCGCCCTGGGCTGCCTGGTCAAGGACTACTTCCCCGAACCGGTGACGGTGTCG
TGGAACTCAGGCGCCCTGACCAGCGGCGTGCACACCTTCCCGGCTGTCCTACAGTCCT
CAGGACTCTACTCCCTCAGCAGCGTGGTGACCGTGCCCTCCAGCAGCTTGGGCACGAA
GACCTACACCTGCAACGTAGATCACAAGCCCAGCAACACCAAGGTGGACAAGAGAGT
TGAGTCCAAATATGGTCCCCCATGCCCACCCTGCCCAGCACCTGAGTTCGAAGGGGGA
CCATCAGTCTTCCTGTTCCCCCCAAAACCCAAGGACACTCTCATGATCTCCCGGACCCC
TGAGGTCACGTGCGTGGTGGTGGACGTGAGCCAGGAAGACCCCGAGGTCCAGTTCAA
CTGGTACGTGGATGGCGTGGAGGTGCATAATGCCAAGACAAAGCCGCGGGAGGAGCA
GTTCAACAGCACGTACCGTGTGGTCAGCGTCCTCACCGTCCTGCACCAGGACTGGCTG
AACGGCAAGGAGTACAAGTGCAAGGTCTCCAACAAAGGCCTCCCGTCCTCCATCGAG
AAAACCATCTCCAAAGCCAAAGGGCAGCCCCGAGAGCCACAGGTGTACACCCTGCCC
CCATCCCAGGAGGAGATGACCAAGAACCAGGTCAGCCTGACCTGCCTGGTCAAAGGC
TTCTACCCCAGCGACATCGCCGTGGAGTGGGAGAGCAATGGGCAGCCGGAGAACAAC
TACAAGACCACGCCTCCCGTGCTGGACTCCGACGGCTCCTTCTTCCTCTACAGCAGGCT
AACCGTGGACAAGAGCAGGTGGCAGGAGGGGAATGTCTTCTCATGCTCCGTGATGCAT
GAGGCTCTGCACAACCACTACACACAGAAGAGCCTCTCCCTGTCTCTGGGTAAAGCTA
GCTGA
Anti-Langerin15B10H-LV-hIgG4H-C (SEQ ID NO: 105):
QVQLRQSGPELVKPGASVKMSCKASGYTFTDYVISWVKQRTGQGLEWIGDIYPGSGYSFY
NENFKGKATLTADKSSTTAYMQLSSLTSEDSAVYFCATYYNYPFAYWGQGTLVTVSAAK
TTGPSVFPLAPCSRSTSESTAALGCLVKDYFPEPVTVSWNSGALTSGVHTFPAVLQSSGLYS
LSSVVTVPSSSLGTKTYTCNVDHKPSNTKVDKRVESKYGPPCPPCPAPEFEGGPSVFLFPPK
PKDTLMISRTPEVTCVVVDVSQEDPEVQFNWYVDGVEVHNAKTKPREEQFNSTYRVVSV
LTVLHQDWLNGKEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDKSRWQEGNVFSCS
VMHEALHNHYTQKSLSLSLGKAS.
Anti-Langerin15B10K-LV-hIgGK-C (SEQ ID NO: 106):
ATGAAGTTGCCTGTTAGGCTGTTGGTGCTGATGTTCTGGATTCCTGCTTCCAGCAGTGA
TGTTGTGATGACCCAAACTCCACTCTCCCTGCCTGTCCGTCTTGGAGATCAAGCCTCCA
TCTCTTGCAGATCTAGTCAGAGCCTTGTACACAGTAATGGAAACACCTATTTACATTGG
TACCTGCAGAAGCCAGGCCAGTCTCCAAAGCTCCTGATCTACAAAGTTTCCAACCGAT
TTTCTGGGGTCCCAGACAGGTTCAGTGGCAGTGGATCAGGGACAAATTTCACACTCAA
GATCAGCAGAGTGGAGGCTGAGGATCTGGGACTTTATTTCTGCTCTCAAAGTACACAT
GTTCCGTACACGTTCGGAGGGGGGACCAAGCTCGAGATCAAACGAACTGTGGCTGCA
CCATCTGTCTTCATCTTCCCGCCATCTGATGAGCAGTTGAAATCTGGAACTGCCTCTGT
TGTGTGCCTGCTGAATAACTTCTATCCCAGAGAGGCCAAAGTACAGTGGAAGGTGGAT
AACGCCCTCCAATCGGGTAACTCCCAGGAGAGTGTCACAGAGCAGGACAGCAAGGAC
AGCACCTACAGCCTCAGCAGCACCCTGACGCTGAGCAAAGCAGACTACGAGAAACAC
AAAGTCTATGCCTGCGAAGTCACCCATCAGGGCCTGAGCTCGCCCGTCACAAAGAGCT
TCAACAGGGGAGAGTGTTAG
Anti-Langerin15B10K-LV-hIgGK-C (SEQ ID NO: 107):
DVVMTQTPLSLPVRLGDQASISCRSSQSLVHSNGNTYLHWYLQKPGQSPKLLIYKVSNRFS
GVPDRFSGSGSGTNFTLKISRVEAEDLGLYFCSQSTHVPYTFGGGTKLEIKRTVAAPSVFIFP
PSDEQLKSGTASVVCLLNNFYPREAKVQWKVDNALQSGNSQESVTEQDSKDSTYSLSSTL
TLSKADYEKHKVYACEVTHQGLSSPVTKSFNRGEC
Anti-Langerin2G3H-LV-hIgG4H-C (SEQ ID NO: 108):
ATGACATTGAACATGCTGTTGGGGCTGAGGTGGGTTTTCTTTGTTGTTTTTTATCAAGG
TGTGCATTGTGAGGTGCAGCTTGTTGAGTCTGGTGGAGGATTGGTGCAGCCTAAAGGG
TCATTGAAACTCTCATGTGCAGCCTCTGGATTAACCTTCAATATCTACGCCATGAACTG
GGTCCGCCAGGCTCCAGGAAAGGGTTTGGAATGGGTTGCTCGCATAAGAAATAAAAG
TAATAATTATGCAACATATTATGCCGATTCAGTGAAAGACAGGTTCACCATCTCCAGA
GATGATTCACAAAGCTTGCTCTATCTGCAAATGAACAACTTGAAAACTGAGGACACAG
CCATGTATTACTGTGTGGGACGGGACTGGTTTGATTACTGGGGCCAAGGGACTCTGGT
CACTGTCTCTGCAGCCAAAACGAAGGGCCCATCCGTCTTCCCCCTGGCGCCCTGCTCC
AGGAGCACCTCCGAGAGCACAGCCGCCCTGGGCTGCCTGGTCAAGGACTACTTCCCCG
AACCGGTGACGGTGTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCCCTCCA
GCAGCTTGGGCACGAAGACCTACACCTGCAACGTAGATCACAAGCCCAGCAACACCA
AGGTGGACAAGAGAGTTGAGTCCAAATATGGTCCCCCATGCCCACCCTGCCCAGCACC
TGAGTTCGAAGGGGGACCATCAGTCTTCCTGTTCCCCCCAAAACCCAAGGACACTCTC
ATGATCTCCCGGACCCCTGAGGTCACGTGCGTGGTGGTGGACGTGAGCCAGGAAGACC
CCGAGGTCCAGTTCAACTGGTACGTGGATGGCGTGGAGGTGCATAATGCCAAGACAA
AGCCGCGGGAGGAGCAGTTCAACAGCACGTACCGTGTGGTCAGCGTCCTCACCGTCCT
GCACCAGGACTGGCTGAACGGCAAGGAGTACAAGTGCAAGGTCTCCAACAAAGGCCT
CCCGTCCTCCATCGAGAAAACCATCTCCAAAGCCAAAGGGCAGCCCCGAGAGCCACA
GGTGTACACCCTGCCCCCATCCCAGGAGGAGATGACCAAGAACCAGGTCAGCCTGAC
CTGCCTGGTCAAAGGCTTCTACCCCAGCGACATCGCCGTGGAGTGGGAGAGCAATGGG
CAGCCGGAGAACAACTACAAGACCACGCCTCCCGTGCTGGACTCCGACGGCTCCTTCT
TCCTCTACAGCAGGCTAACCGTGGACAAGAGCAGGTGGCAGGAGGGGAATGTCTTCTC
ATGCTCCGTGATGCATGAGGCTCTGCACAACCACTACACACAGAAGAGCCTCTCCCTG
TCTCTGGGTAAAGCTAGCTGA
Anti-Langerin2G3H-LV-hIgG4H-C (SEQ ID NO: 109):
MTLNMLLGLRWVFFVVFYQGVHCEVQLVESGGGLVQPKGSLKLSCAASGLTFNIYAMN
WVRQAPGKGLEWVARIRNKSNNYATYYADSVKDRFTISRDDSQSLLYLQMNNLKTEDTA
MYYCVGRDWFDYWGQGTLVTVSAAKTKGPSVFPLAPCSRSTSESTAALGCLVKDYFPEP
VTVSWNSGALTSGVHTFPAVLQSSGLYSLSSVVTVPSSSLGTKTYTCNVDHKPSNTKVDK
RVESKYGPPCPPCPAPEFEGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIEKTIS
KAKGQPREPQVYTLPPSQEEMTKNQVSLTCLVKGFYPSDIAVEWESNGQPENNYKTTPPV
LDSDGSFFLYSRLTVDKSRWQEGNVFSCSVMHEALHNHYTQKSLSLSLGKAS.
Anti-Langerin2G3L-LV-hIgGK-C (SEQ ID NO: 110):
ATGGCCTGGATTTCACTTATACTCTCTCTCCTGGCTCTCAGCTCAGGGGCCATTTCCCA
GGCTGTTGTGACTCAGGAATCTGCACTCACCACATCACCTGGTGAAACAGTCACACTC
ACTTGTCGCTCAAGTACTGGGGCTGTTACAACTAGTAACTATGCCAACTGGGTCCAAG
AAAAACCAGATCATTTATTCACTGGTCTAATAGGTGGTACCAACAACCGAGTTTCAGG
TGTTCCTGCCAGATTCTCAGGCTCCCTGATTGGAGACAAGGCTGCCCTCACCATCACA
GGGGCACAGACTGAGGATGAGGCAATATATTTCTGTGCTCTATGGTACAGCAACCATT
GGGTGTTCGGTGGAGGAACCAAACTCGAGATCAAACGAACTGTGGCTGCACCATCTGT
CTTCATCTTCCCGCCATCTGATGAGCAGTTGAAATCTGGAACTGCCTCTGTTGTGTGCC
TGCTGAATAACTTCTATCCCAGAGAGGCCAAAGTACAGTGGAAGGTGGATAACGCCCT
CCAATCGGGTAACTCCCAGGAGAGTGTCACAGAGCAGGACAGCAAGGACAGCACCTA
CAGCCTCAGCAGCACCCTGACGCTGAGCAAAGCAGACTACGAGAAACACAAAGTCTA
TGCCTGCGAAGTCACCCATCAGGGCCTGAGCTCGCCCGTCACAAAGAGCTTCAACAGG
GGAGAGTGTTAG
Anti-Langerin2G3L-LV-hIgGK-C (SEQ ID NO: 111):
MAWISLILSLLALSSGAISQAVVTQESALTTSPGETVTLTCRSSTGAVTTSNYANWVQEKP
DHLFTGLIGGTNNRVSGVPARFSGSLIGDKAALTITGAQTEDEAIYFCALWYSNHWVFGG
GTKLEIKRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC
Anti-Lox_1_10F9H-LV-hIgG4H-C (SEQ ID NO: 112):
ATGGAATGGACCTGGGTCTTTCTCTTCCTCCTGTCAGTAACTGCAGGTGTCCACTCCCA
GGTTCAGCTGCAGCAGTCTGGAGCTGAGCTGATGAAGCCTGGGGCCTCAGTGAAGATA
TCCTGCAAGGCTACTGGCTACACATTCGGTAGCTACTGGATAGAGTGGGTAAAGCAGA
GGCCTGGACATGGCCTTGAGTGGATTGGAGAGATTTTACCTGGAAGTGGTAATACTAA
CTACAATGAGAACTTCAAGGGCAAGGCCACATTCACTGCAGATACATCCTCCAACACA
GCCTACATGCAACTCACCAGTCTGACATCTGAGGACTCTGCCGTCTATTACTGTGCTAG
GGCGGGGATTTATTGGGGCCAAGGGACTCTGGTCACTGTCTCTGCAGCCAAAACGAAG
GGCCCATCCGTCTTCCCCCTGGCGCCCTGCTCCAGGAGCACCTCCGAGAGCACAGCCG
CCCTGGGCTGCCTGGTCAAGGACTACTTCCCCGAACCGGTGACGGTGTCGTGGAACTC
AGGCGCCCTGACCAGCGGCGTGCACACCTTCCCGGCTGTCCTACAGTCCTCAGGACTC
TACTCCCTCAGCAGCGTGGTGACCGTGCCCTCCAGCAGCTTGGGCACGAAGACCTACA
CCTGCAACGTAGATCACAAGCCCAGCAACACCAAGGTGGACAAGAGAGTTGAGTCCA
AATATGGTCCCCCATGCCCACCCTGCCCAGCACCTGAGTTCGAAGGGGGACCATCAGT
CTTCCTGTTCCCCCCAAAACCCAAGGACACTCTCATGATCTCCCGGACCCCTGAGGTCA
CGTGCGTGGTGGTGGACGTGAGCCAGGAAGACCCCGAGGTCCAGTTCAACTGGTACGT
GGATGGCGTGGAGGTGCATAATGCCAAGACAAAGCCGCGGGAGGAGCAGTTCAACAG
CACGTACCGTGTGGTCAGCGTCCTCACCGTCCTGCACCAGGACTGGCTGAACGGCAAG
GAGTACAAGTGCAAGGTCTCCAACAAAGGCCTCCCGTCCTCCATCGAGAAAACCATCT
CCAAAGCCAAAGGGCAGCCCCGAGAGCCACAGGTGTACACCCTGCCCCCATCCCAGG
AGGAGATGACCAAGAACCAGGTCAGCCTGACCTGCCTGGTCAAAGGCTTCTACCCCAG
CGACATCGCCGTGGAGTGGGAGAGCAATGGGCAGCCGGAGAACAACTACAAGACCAC
GCCTCCCGTGCTGGACTCCGACGGCTCCTTCTTCCTCTACAGCAGGCTAACCGTGGACA
AGAGCAGGTGGCAGGAGGGGAATGTCTTCTCATGCTCCGTGATGCATGAGGCTCTGCA
CAACCACTACACACAGAAGAGCCTCTCCCTGTCTCTGGGTAAAGCTAGCTGA
Anti-Lox_1_10F9H-LV-hIgG4H-C (SEQ ID NO: 113):
MEWTWVFLFLLSVTAGVHSQVQLQQSGAELMKPGASVKISCKATGYTFGSYWIEWVKQ
RPGHGLEWIGEILPGSGNTNYNENFKGKATFTADTSSNTAYMQLTSLTSEDSAVYYCARA
GIYWGQGTLVTVSAAKTKGPSVFPLAPCSRSTSESTAALGCLVKDYFPEPVTVSWNSGALT
SGVHTFPAVLQSSGLYSLSSVVTVPSSSLGTKTYTCNVDHKPSNTKVDKRVESKYGPPCPP
CPAPEFEGGPSVFLFPPKPKDTLMISRTPEVTCVVVDVSQEDPEVQFNWYVDGVEVHNAK
TKPREEQFNSTYRVVSVLTVLHQDWLNGKEYKCKVSNKGLPSSIEKTISKAKGQPREPQV
YTLPPSQEEMTKNQVSLTCLVKGFYPSDIAVEWESNGQPENNYKTTPPVLDSDGSFFLYSR
LTVDKSRWQEGNVFSCSVMHEALHNHYTQKSLSLSLGKAS
Anti-Lox_1_10F9K-LV-hIgGK-C (SEQ ID NO: 114):
ATGGAGAAAGACACACTCCTGCTATGGGTCCTGCTTCTCTGGGTTCCAGGTTCCACAG
GTGACATTGTGCTGACCCAATCTCCAGCTTTTTTGGCTGTGTCTCTAGGGCAGAGGGCC
ACCATCTCCTGCAGAGCCAGCGAAAGTGTTGATAATTATGGCATTAGTTTTATGAACT
GGTTCCAACAGAAACCAGGACAGCCACCCAAACTCCTCATCTATGTTGCATCCAAGCA
AGGATCCGGGGTCCCTGCCAGGTTTAGTGGCAGTGGGTCTGGGACAGACTTCAGCCTC
AACATCCATCCTATGGAGGAGGATGATACTGCAATGTATTTCTGTCAGCAAAGTAAGG
AGGTTCCTCGGACGTTCGGTGGAGGCACCAAGCTCGAGATCAAACGAACTGTGGCTGC
ACCATCTGTCTTCATCTTCCCGCCATCTGATGAGCAGTTGAAATCTGGAACTGCCTCTG
TTGTGTGCCTGCTGAATAACTTCTATCCCAGAGAGGCCAAAGTACAGTGGAAGGTGGA
TAACGCCCTCCAATCGGGTAACTCCCAGGAGAGTGTCACAGAGCAGGACAGCAAGGA
CAGCACCTACAGCCTCAGCAGCACCCTGACGCTGAGCAAAGCAGACTACGAGAAACA
CAAAGTCTATGCCTGCGAAGTCACCCATCAGGGCCTGAGCTCGCCCGTCACAAAGAGC
TTCAACAGGGGAGAGTGTTAG
Anti-Lox_1_10F9K-LV-hIgGK-C (SEQ ID NO: 115):
MEKDTLLLWVLLLWVPGSTGDIVLTQSPAFLAVSLGQRATISCRASESVDNYGISFMNWF
QQKPGQPPKLLIYVASKQGSGVPARFSGSGSGTDFSLNIHPMEEDDTAMYFCQQSKEVPRT
FGGGTKLEIKRTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSFNRGEC
Anti-LOX-111C8H-LV-hIgG4H-C (SEQ ID NO: 116):
ATGGAATGTAACTGGATACTTCCTTTTATTCTGTCGGTAACTTCAGGGGTCTACTCAGA
GGTTCAGCTCCAGCAGTCTGGGACTGTGCTGGCAAGGCCTGGGGCTTCAGTGAAGATG
TCCTGCAAGGCTTCTGGCTACACCTTTACCAGCTACTGGATGCACTGGGTAAAACAGA
GGCCTGGACAGGGTCTGGAATGGATTGGCGCTATTTATCCTGGAAATAGTGATACTAC
CTACAACCAGAAGTTCAAGGGCAAGGCCAAACTGACTGCAGTCACATCCACCAGCAC
TGCCTACATGGAGCTCAGCAGCCTGACAAATGAGGACTCTGCGGTCTATTACTGTACA
CCTACTTACTACTTTGACTACTGGGGCCAAGGCACCTCTCTCACAGTCTCCTCAGCCAA
AACGAAGGGCCCATCCGTCTTCCCCCTGGCGCCCTGCTCCAGGAGCACCTCCGAGAGC
ACAGCCGCCCTGGGCTGCCTGGTCAAGGACTACTTCCCCGAACCGGTGACGGTGTCGT
GGAACTCAGGCGCCCTGACCAGCGGCGTGCACACCTTCCCGGCTGTCCTACAGTCCTC
AGGACTCTACTCCCTCAGCAGCGTGGTGACCGTGCCCTCCAGCAGCTTGGGCACGAAG
ACCTACACCTGCAACGTAGATCACAAGCCCAGCAACACCAAGGTGGACAAGAGAGTT
GAGTCCAAATATGGTCCCCCATGCCCACCCTGCCCAGCACCTGAGTTCGAAGGGGGAC
CATCAGTCTTCCTGTTCCCCCCAAAACCCAAGGACACTCTCATGATCTCCCGGACCCCT
GAGGTCACGTGCGTGGTGGTGGACGTGAGCCAGGAAGACCCCGAGGTCCAGTTCAAC
TGGTACGTGGATGGCGTGGAGGTGCATAATGCCAAGACAAAGCCGCGGGAGGAGCAG
TTCAACAGCACGTACCGTGTGGTCAGCGTCCTCACCGTCCTGCACCAGGACTGGCTGA
ACGGCAAGGAGTACAAGTGCAAGGTCTCCAACAAAGGCCTCCCGTCCTCCATCGAGA
AAACCATCTCCAAAGCCAAAGGGCAGCCCCGAGAGCCACAGGTGTACACCCTGCCCC
CATCCCAGGAGGAGATGACCAAGAACCAGGTCAGCCTGACCTGCCTGGTCAAAGGCT
TCTACCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAGGCT
AACCGTGGACAAGAGCAGGTGGCAGGAGGGGAATGTCTTCTCATGCTCCGTGATGCAT
GAGGCTCTGCACAACCACTACACACAGAAGAGCCTCTCCCTGTCTCTGGGTAAAGCTA
GCTGA
Anti-LOX-111C8H-LV-hIgG4H-C (SEQ ID NO: 117):
MECNWILPFILSVTSGVYSEVQLQQSGTVLARPGASVKMSCKASGYTFTSYWMHWVKQR
PGQGLEWIGAIYPGNSDTTYNQKFKGKAKLTAVTSTSTAYMELSSLTNEDSAVYYCTPTY
YFDYWGQGTSLTVSSAKTKGPSVFPLAPCSRSTSESTAALGCLVKDYFPEPVTVSWNSGAL
TSGVHTFPAVLQSSGLYSLSSVVTVPSSSLGTKTYTCNVDHKPSNTKVDKRVESKYGPPCP
PCPAPEFEGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPREPQ
VYTLPPSQEEMTKNQVSLTCLVKGFYPSDIAVEWESNGQPENNYKTTPPVLDSDGSFFLYS
RLTVDKSRWQEGNVFSCSVMHEALHNHYTQKSLSLSLGKAS
Anti-LOX-111C8K-LV-hIgGK-C (SEQ ID NO: 118):
ATGAGTCCTGCCCAATTCCTGTTTCTGTTAGTGCTCTGGATTCGGGAAACCAACGGTGA
TGTTGTGATGACCCAGACTCCACTCACTTTGTCGGTTACCATTGGACAACCAGCCTCCA
TCTCTTGCAAGTCAAGTCAGAGCCTCTTAGATAGTGATGGAAAGACATATTTGAATTG
GTTCTTACAGAGGCCAGGCCAGTCTCCAAAGCGCCTAATCTATCTGGTGTCTAAACTG
GACTCTGGAGTCCCTGACAGGTTCACTGGCAGTGGATCAGGGACAGATTTCACACTGA
AAATCAGCAGAGTGGAGGCTGAGGATTTGGGAGTTTATTATTGCTGGCAAGGTACACA
TTTTCCGTGGACGTTCGGTGGAGGCACCAAGCTCGAGATCAAACGAACTGTGGCTGCA
CCATCTGTCTTCATCTTCCCGCCATCTGATGAGCAGTTGAAATCTGGAACTGCCTCTGT
TGTGTGCCTGCTGAATAACTTCTATCCCAGAGAGGCCAAAGTACAGTGGAAGGTGGAT
AACGCCCTCCAATCGGGTAACTCCCAGGAGAGTGTCACAGAGCAGGACAGCAAGGAC
AGCACCTACAGCCTCAGCAGCACCCTGACGCTGAGCAAAGCAGACTACGAGAAACAC
AAAGTCTATGCCTGCGAAGTCACCCATCAGGGCCTGAGCTCGCCCGTCACAAAGAGCT
TCAACAGGGGAGAGTGTTAG
Anti-LOX-111C8K-LV-hIgGK-C (SEQ ID NO: 119):
MSPAQFLFLLVLWIRETNGDVVMTQTPLTLSVTIGQPASISCKSSQSLLDSDGKTYLNWFL
QRPGQSPKRLIYLVSKLDSGVPDRFTGSGSGTDFTLKISRVEAEDLGVYYCWQGTHFPWTF
GGGTKLEIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
Anti-LOX-115C4H-LV-hIgG4H-C (SEQ ID NO: 120):
ATGGGAGGGATCTGGATCTTTCTCTTCCTCCTGTCAGGAACTGCAGGTGCCCACTCTGA
GATCCAGCTGCAGCAGACTGGACCTGAGCTGGTGAAGCCTGGGGCTTCAGTGAAGAT
ATCCTGCAAGGCTTCTGGTTATCCATTCACTGACTACATCATGGTCTGGGTGAAGCAG
AGCCATGGAAAGAGCCTTGAGTGGATTGGAAATATTAGTCCTTACTATGGTACTACTA
ACTACAATCTGAAGTTCAAGGGCAAGGCCACATTGACTGTAGACAAATCTTCCAGCAC
AGCCTACATGCAGCTCAACAGTCTGACATCTGAGGACTCTGCAGTCTATTACTGTGCA
AGATCCCCTAACTGGGACGGGGCCTGGTTTGCTCACTGGGGCCAAGGGGCTCTGGTCA
CTGTCTCTGCAGCCAAAACAAAGGGCCCATCCGTCTTCCCCCTGGCGCCCTGCTCCAG
GAGCACCTCCGAGAGCACAGCCGCCCTGGGCTGCCTGGTCAAGGACTACTTCCCCGAA
CCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGC
AGCTTGGGCACGAAGACCTACACCTGCAACGTAGATCACAAGCCCAGCAACACCAAG
GTGGACAAGAGAGTTGAGTCCAAATATGGTCCCCCATGCCCACCCTGCCCAGCACCTG
AGTTCGAAGGGGGACCATCAGTCTTCCTGTTCCCCCCAAAACCCAAGGACACTCTCAT
GATCTCCCGGACCCCTGAGGTCACGTGCGTGGTGGTGGACGTGAGCCAGGAAGACCCC
GAGGTCCAGTTCAACTGGTACGTGGATGGCGTGGAGGTGCATAATGCCAAGACAAAG
CCGCGGGAGGAGCAGTTCAACAGCACGTACCGTGTGGTCAGCGTCCTCACCGTCCTGC
ACCAGGACTGGCTGAACGGCAAGGAGTACAAGTGCAAGGTCTCCAACAAAGGCCTCC
CGTCCTCCATCGAGAAAACCATCTCCAAAGCCAAAGGGCAGCCCCGAGAGCCACAGG
TGTACACCCTGCCCCCATCCCAGGAGGAGATGACCAAGAACCAGGTCAGCCTGACCTG
CCTGGTCAAAGGCTTCTACCCCAGCGACATCGCCGTGGAGTGGGAGAGCAATGGGCA
GCCGGAGAACAACTACAAGACCACGCCTCCCGTGCTGGACTCCGACGGCTCCTTCTTC
CTCTACAGCAGGCTAACCGTGGACAAGAGCAGGTGGCAGGAGGGGAATGTCTTCTCA
TGCTCCGTGATGCATGAGGCTCTGCACAACCACTACACACAGAAGAGCCTCTCCCTGT
CTCTGGGTAAAGCTAGCTGATTAATTAA
Anti-LOX-115C4H-LV-hIgG4H-C (SEQ ID NO: 121):
MGGIWIFLFLLSGTAGAHSEIQLQQTGPELVKPGASVKISCKASGYPFTDYIMVWVKQSHG
KSLEWIGNISPYYGTTNYNLKFKGKATLTVDKSSSTAYMQLNSLTSEDSAVYYCARSPNW
DGAWFAHWGQGALVTVSAAKTKGPSVFPLAPCSRSTSESTAALGCLVKDYFPEPVTVSW
NSGALTSGVHTFPAVLQSSGLYSLSSVVTVPSSSLGTKTYTCNVDHKPSNTKVDKRVESKY
GPPCPPCPAPEFEGGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYKTTPPVLDSDGSF
FLYSRLTVDKSRWQEGNVFSCSVMHEALHNHYTQKSLSLSLGKAS
Anti-LOX-115C4K-LV-hIgGK-C (SEQ ID NO: 122):
ATGGAGACAGACACAATCCTGCTATGGGTGCTGCTGCTCTGGGTTCCAGGCTCCACTG
GTGACATTGTGCTGACCCAATCTCCAGCTTCTTTGGCTGTGTCTCTAGGGCAGAGGGCC
ACCATCTCCTGCAAGGCCAGCCAAAGTGTTGATTATGATGGTGATAGTTATATGAACT
GGTTCCAACAGAAACCAGGACAGCCACCCAAACTCCTCATCTATGCTGCATCCAATCT
AGAATCTGGGATCCCAGCCAGGTTTAGTGGCAGTGGGTCTGGGACAGACTTCACCCTC
AACATCCATCCTGTGGAGGAGGAGGATGCTGCAACCTATTACTGTCAGCAAAGTAATG
AGGATCCATTCACGTTCGGCTCGGGGACAAAGCTCGAGATCAAACGAACTGTGGCTGC
ACCATCTGTCTTCATCTTCCCGCCATCTGATGAGCAGTTGAAATCTGGAACTGCCTCTG
TTGTGTGCCTGCTGAATAACTTCTATCCCAGAGAGGCCAAAGTACAGTGGAAGGTGGA
TAACGCCCTCCAATCGGGTAACTCCCAGGAGAGTGTCACAGAGCAGGACAGCAAGGA
CAGCACCTACAGCCTCAGCAGCACCCTGACGCTGAGCAAAGCAGACTACGAGAAACA
CAAAGTCTATGCCTGCGAAGTCACCCATCAGGGCCTGAGCTCGCCCGTCACAAAGAGC
TTCAACAGGGGAGAGTGTTAG
Anti-LOX-115C4K-LV-hIgGK-C (SEQ ID NO: 123):
METDTILLWVLLLWVPGSTGDIVLTQSPASLAVSLGQRATISCKASQSVDYDGDSYMNWF
QQKPGQPPKLLIYAASNLESGIPARFSGSGSGTDFTLNIHPVEEEDAATYYCQQSNEDPFTF
GSGTKLEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC
Anti-Marco_10B7.3G4H-LV-hIgG4H-C (SEQ ID NO: 124):
ATGGCTGTCCTGGGGCTGCTTCTCTGCCTGGTGACGTTCCCAAGCTGTGTCCTGTCCCA
GGTGCAGCTGAAGGAGTCAGGACCTGGCCTGGTGGCACCCTCACAGAGCCTGTCCATC
ACATGCACTGTCTCTGGGTTCTCATTATCCAGATATAGTGTATTTTGGGTTCGCCAGCC
TCCAGGAAAGGGTCTGGAGTGGCTGGGATTGATATGGGGTGGTGGAAGCACAGACTA
TAATTCAGCTCTCAAATCCAGACTGAGCATCAGCAAGGACAACTCCAAGAGCCAAGTT
TTCTTAAAAATGAACAGTCTGCAAACTGATGACACAGCCATGTACTACTGTGCCAGAA
TCTACTTTGATTACGACGGGGCTATGGACTACTGGGGTCAAGGAACCTCAGTCACCGT
CTCCTCAGCCAAAACAACGGGCCCATCCGTCTTCCCCCTGGCGCCCTGCTCCAGGAGC
ACCTCCGAGAGCACAGCCGCCCTGGGCTGCCTGGTCAAGGACTACTTCCCCGAACCGG
TGACGGTGTCGTGGAACTCAGGCGCCCTGACCAGCGGCGTGCACACCTTCCCGGCTGT
CCTACAGTCCTCAGGACTCTACTCCCTCAGCAGCGTGGTGACCGTGCCCTCCAGCAGC
TTGGGCACGAAGACCTACACCTGCAACGTAGATCACAAGCCCAGCAACACCAAGGTG
GACAAGAGAGTTGAGTCCAAATATGGTCCCCCATGCCCACCCTGCCCAGCACCTGAGT
TCGAAGGGGGACCATCAGTCTTCCTGTTCCCCCCAAAACCCAAGGACACTCTCATGAT
CTCCCGGACCCCTGAGGTCACGTGCGTGGTGGTGGACGTGAGCCAGGAAGACCCCGA
GGTCCAGTTCAACTGGTACGTGGATGGCGTGGAGGTGCATAATGCCAAGACAAAGCC
GCGGGAGGAGCAGTTCAACAGCACGTACCGTGTGGTCAGCGTCCTCACCGTCCTGCAC
CAGGACTGGCTGAACGGCAAGGAGTACAAGTGCAAGGTCTCCAACAAAGGCCTCCCG
TCCTCCATCGAGAAAACCATCTCCAAAGCCAAAGGGCAGCCCCGAGAGCCACAGGTG
TACACCCTGCCCCCATCCCAGGAGGAGATGACCAAGAACCAGGTCAGCCTGACCTGCC
TGGTCAAAGGCTTCTACCCCAGCGACATCGCCGTGGAGTGGGAGAGCAATGGGCAGC
CGGAGAACAACTACAAGACCACGCCTCCCGTGCTGGACTCCGACGGCTCCTTCTTCCT
CTACAGCAGGCTAACCGTGGACAAGAGCAGGTGGCAGGAGGGGAATGTCTTCTCATG
CTCCGTGATGCATGAGGCTCTGCACAACCACTACACACAGAAGAGCCTCTCCCTGTCT
CTGGGTAAAGCTAGCTG
Anti-Marco_10B7.3G4H-LV-hIgG4H-C (SEQ ID NO: 125):
MAVLGLLLCLVTFPSCVLSQVQLKESGPGLVAPSQSLSITCTVSGFSLSRYSVFWVRQPPG
KGLEWLGLIWGGGSTDYNSALKSRLSISKDNSKSQVFLKMNSLQTDDTAMYYCARIYFDY
DGAMDYWGQGTSVTVSSAKTTGPSVFPLAPCSRSTSESTAALGCLVKDYFPEPVTVSWNS
GALTSGVHTFPAVLQSSGLYSLSSVVTVPSSSLGTKTYTCNVDHKPSNTKVDKRVESKYGP
PCPPCPAPEFEGGPSVFLFPPKPKDTLMISRTPEVTCVVVDVSQEDPEVQFNWYVDGVEVH
NAKTKPREEQFNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTPPVLDSDGSFFL
YSRLTVDKSRWQEGNVFSCSVMHEALHNHYTQKSLSLSLGKAS
AntiMarco_10B7.3G4K_H-V-hIgGK-C (SEQ ID NO: 126):
ATGCATCGCACCAGCATGGGCATCAAGATGGAGTCACGGATTCAGGCATTTGTATTCG
TGTTTCTCTGGTTGTCTGGTGTTGGCGGAGACATTGTGATGACCCAGTCTCACAAATTC
ATGTCCACATCAGTAGGAGACAGGGTCAGCGTCACCTGCAAGGCCAGTCAGGATGTG
ACTTCTGCTGTAGCCTGGTATCAACAAAAACCAGGGCAATCTCCTAAACTACTGATTT
ACTGGGCATCCACCCGGCACACTGGAGTCCCTGATCGCTTCACAGGCAGTGGATCTGG
GACAGATTATACTCTCACCATCAGCAGTGGGCAGGCTGAAGACCTGGCACTTTATTAC
TGTCACCAATATTATAGCGCTCCTCGGACGTTCGGTGGAGGCACCAAGCTCGAGATCA
AACGAACTGTGGCTGCACCATCTGTCTTCATCTTCCCGCCATCTGATGAGCAGTTGAAA
TCTGGAACTGCCTCTGTTGTGTGCCTGCTGAATAACTTCTATCCCAGAGAGGCCAAAGT
ACAGTGGAAGGTGGATAACGCCCTCCAATCGGGTAACTCCCAGGAGAGTGTCACAGA
GCAGGACAGCAAGGACAGCACCTACAGCCTCAGCAGCACCCTGACGCTGAGCAAAGC
AGACTACGAGAAACACAAAGTCTATGCCTGCGAAGTCACCCATCAGGGCCTGAGCTC
GCCCGTCACAAAGAGCTTCAACAGGGGAGAGTGTTAG
AntiMarco_10B7.3G4K_H-V-hIgGK-C (SEQ ID NO: 127):
MHRTSMGIKMESRIQAFVFVFLWLSGVGGDIVMTQSHKFMSTSVGDRVSVTCKASQDVT
SAVAWYQQKPGQSPKLLIYWASTRHTGVPDRFTGSGSGTDYTLTISSGQAEDLALYYCHQ
YYSAPRTFGGGTKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSPVTKSFNR
GEC
Anti-Marco_11A8.3C9_H-V-hIgG4H-C (SEQ ID NO: 128):
ATGGAATGGAACTGGGTCGTTCTCTTCCTCCTGTCATTAACTGCAGGTGTCTATGCCCA
GGGTCAGATGCAGCAGTCTGGAGCTGAGCTGGTGAAGCCTGGGGCTTCAGTGAAGCT
GTCCTGCAAGACTTCTGGCTTCACCTTCAGCAGTAACTATATAAGTTGGTTGAAGCAA
AAGCCTGGACAGAGTCTTGAGTGGATTGCATGGATTTATGCTGGAACTGGTGGTATTA
CCTATAATCAGAAGTTCAGAGGCAGGGCCCAACTGACTGTAGACACATCCTCCAGCAC
AGCCTACATGCAGTTCAGCAGCCTGACAACTGATGACTCTGCCATCTATTACTGTGCA
AGACACGTGAGGGGTTACCATCCTATGGACTACTGGGGTCAAGGAACCTCAGTCACCG
TCTCCTCAGCCAAAACGAAGGGCCCATCCGTCTTCCCCCTGGCGCCCTGCTCCAGGAG
CACCTCCGAGAGCACAGCCGCCCTGGGCTGCCTGGTCAAGGACTACTTCCCCGAACCG
GTGACGGTGTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCAGCAG
CTTGGGCACGAAGACCTACACCTGCAACGTAGATCACAAGCCCAGCAACACCAAGGT
GGACAAGAGAGTTGAGTCCAAATATGGTCCCCCATGCCCACCCTGCCCAGCACCTGAG
TTCGAAGGGGGACCATCAGTCTTCCTGTTCCCCCCAAAACCCAAGGACACTCTCATGA
TCTCCCGGACCCCTGAGGTCACGTGCGTGGTGGTGGACGTGAGCCAGGAAGACCCCGA
GGTCCAGTTCAACTGGTACGTGGATGGCGTGGAGGTGCATAATGCCAAGACAAAGCC
GCGGGAGGAGCAGTTCAACAGCACGTACCGTGTGGTCAGCGTCCTCACCGTCCTGCAC
CAGGACTGGCTGAACGGCAAGGAGTACAAGTGCAAGGTCTCCAACAAAGGCCTCCCG
TCCTCCATCGAGAAAACCATCTCCAAAGCCAAAGGGCAGCCCCGAGAGCCACAGGTG
TACACCCTGCCCCCATCCCAGGAGGAGATGACCAAGAACCAGGTCAGCCTGACCTGCC
TGGTCAAAGGCTTCTACCCCAGCGACATCGCCGTGGAGTGGGAGAGCAATGGGCAGC
CGGAGAACAACTACAAGACCACGCCTCCCGTGCTGGACTCCGACGGCTCCTTCTTCCT
CTACAGCAGGCTAACCGTGGACAAGAGCAGGTGGCAGGAGGGGAATGTCTTCTCATG
CTCCGTGATGCATGAGGCTCTGCACAACCACTACACACAGAAGAGCCTCTCCCTGTCT
CTGGGTAAAGCTAGCTGA
Anti-Marco_11A8.3C9_H-V-hIgG4H-C (SEQ ID NO: 129):
MEWNWVVLFLLSLTAGVYAQGQMQQSGAELVKPGASVKLSCKTSGFTFSSNYISWLKQK
PGQSLEWIAWIYAGTGGITYNQKFRGRAQLTVDTSSSTAYMQFSSLTTDDSAIYYCARHV
RGYHPMDYWGQGTSVTVSSAKTKGPSVFPLAPCSRSTSESTAALGCLVKDYFPEPVTVSW
NSGALTSGVHTFPAVLQSSGLYSLSSVVTVPSSSLGTKTYTCNVDHKPSNTKVDKRVESKY
GPPCPPCPAPEFEGGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYKTTPPVLDSDGSF
FLYSRLTVDKSRWQEGNVFSCSVMHEALHNHYTQKSLSLSLGKAS
Anti-Marco_11A8.3C9_H-V-hIgGK-C (SEQ ID NO: 130):
ATGGAGTCACAGACTCAGGTCTTTGTATACATGTTGCTGTGGTTGTCTGGTGTTGATGG
AGACATTGTGATGACCCAGTCTCAAAAATTCATGTCCGCATCAGTAGGGGACAGGGTC
AGCGTCACCTGCAGGGCCAGTCAGAATGTGGTTACTAATGTAGGCTGGTATCAACAGA
AACCAGGGCAATCTCCTAAAGTACTGATTTACTCGGCATCCTTCCGGTACAGTGGAGT
CCCTGATCGCTTCACAGGCAGTGGATCTGGGACAGATTTCACTCTCACCATCACCAAT
GTGCAGTCTGAAGACTTGGCAGAGTATTTCTGTCAGCAATATAACAACTATCCGTACA
CGTTCGGAGGGGGGACCAAGCTCGAGATCAAACGAACTGTGGCTGCACCATCTGTCTT
CATCTTCCCGCCATCTGATGAGCAGTTGAAATCTGGAACTGCCTCTGTTGTGTGCCTGC
TGAATAACTTCTATCCCAGAGAGGCCAAAGTACAGTGGAAGGTGGATAACGCCCTCCA
ATCGGGTAACTCCCAGGAGAGTGTCACAGAGCAGGACAGCAAGGACAGCACCTACAG
CCTCAGCAGCACCCTGACGCTGAGCAAAGCAGACTACGAGAAACACAAAGTCTATGC
CTGCGAAGTCACCCATCAGGGCCTGAGCTCGCCCGTCACAAAGAGCTTCAACAGGGG
AGAGTGTTAG
Anti-Marco_11A8.3C9_H-V-hIgGK-C (SEQ ID NO: 131):
MESQTQVFVYMLLWLSGVDGDIVMTQSQKFMSASVGDRVSVTCRASQNVVTNVGWYQ
QKPGQSPKVLIYSASFRYSGVPDRFTGSGSGTDFTLTITNVQSEDLAEYFCQQYNNYPYTF
GGGTKLEIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
Anti-Marco_3H10.1F3_H-V-hIgG4H-C (SEQ ID NO: 132):
ATGGGATGGAGCTATATCATCCTCTTTTTGGTAGCAACAGCTACAGATGTCCACTCCCA
GGTCCAACTGCAGCAGCCTGGGGCTGAACTGGTGAAGCCTGGGGCTTCAGTGAAGCTG
TCCTGCAAGGCTTCTGGCTACACCTTCACCAGCTACTGGATGCACTGGGTGAAGCAGA
GGCCTGGAGAAGGCCTTGAGTGGATTGGAGAGATTAATCCTAGCTACGGTCGTACTGA
CTACAATGGGAAGTTCAAGAACAAGGCCACACTGACTGTAGCCAAATCCTCCAGCAC
AGCCTACATGCAACTCAGCAGCCTGACATCTGAGGACTCTGCGGTCTATTACTGTGCA
AGAGGAGATTACTACGGTAGTAGCTCGTTTGCTTACTGGGGCCAAGGGACTCTGGTCA
CTGTCTCTGCAGCCAAAACAAAGGGCCCATCCGTCTTCCCCCTGGCGCCCTGCTCCAG
GAGCACCTCCGAGAGCACAGCCGCCCTGGGCTGCCTGGTCAAGGACTACTTCCCCGAA
CCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGC
AGCTTGGGCACGAAGACCTACACCTGCAACGTAGATCACAAGCCCAGCAACACCAAG
GTGGACAAGAGAGTTGAGTCCAAATATGGTCCCCCATGCCCACCCTGCCCAGCACCTG
AGTTCGAAGGGGGACCATCAGTCTTCCTGTTCCCCCCAAAACCCAAGGACACTCTCAT
GATCTCCCGGACCCCTGAGGTCACGTGCGTGGTGGTGGACGTGAGCCAGGAAGACCCC
GAGGTCCAGTTCAACTGGTACGTGGATGGCGTGGAGGTGCATAATGCCAAGACAAAG
CCGCGGGAGGAGCAGTTCAACAGCACGTACCGTGTGGTCAGCGTCCTCACCGTCCTGC
ACCAGGACTGGCTGAACGGCAAGGAGTACAAGTGCAAGGTCTCCAACAAAGGCCTCC
CGTCCTCCATCGAGAAAACCATCTCCAAAGCCAAAGGGCAGCCCCGAGAGCCACAGG
TGTACACCCTGCCCCCATCCCAGGAGGAGATGACCAAGAACCAGGTCAGCCTGACCTG
CCTGGTCAAAGGCTTCTACCCCAGCGACATCGCCGTGGAGTGGGAGAGCAATGGGCA
GCCGGAGAACAACTACAAGACCACGCCTCCCGTGCTGGACTCCGACGGCTCCTTCTTC
CTCTACAGCAGGCTAACCGTGGACAAGAGCAGGTGGCAGGAGGGGAATGTCTTCTCA
TGCTCCGTGATGCATGAGGCTCTGCACAACCACTACACACAGAAGAGCCTCTCCCTGT
CTCTGGGTAAAGCTAGCTGA
Anti-Marco_3H10.1F3_H-V-hIgG4H-C (SEQ ID NO: 133):
MGWSYIILFLVATATDVHSQVQLQQPGAELVKPGASVKLSCKASGYTFTSYWMHWVKQ
RPGEGLEWIGEINPSYGRTDYNGKFKNKATLTVAKSSSTAYMQLSSLTSEDSAVYYCARG
DYYGSSSFAYWGQGTLVTVSAAKTKGPSVFPLAPCSRSTSESTAALGCLVKDYFPEPVTVS
WNSGALTSGVHTFPAVLQSSGLYSLSSVVTVPSSSLGTKTYTCNVDHKPSNTKVDKRVES
KYGPPCPPCPAPEFEGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGKAS
Anti-Marco_3H10.1F3_K-V-hIgGK-C (SEQ ID NO: 134):
ATGGAGTCACAGACTCAGGTCTTTGTATACATGTTGCTGTGGTTGTCTGGTGTTGATGG
AGACATTGTGATGACCCAGTCTCAAAAATTCATGTCCACATCATTAGGAGACAGGGTC
AGCGTCACCTGCAAGGCCAGTCAGAATGTGGGTACTAATGTAGCCTGGTATCAACAGA
AACCAGGGCACTCTCCTAAAGCACTGATTTACTCGGCATCCTACCGGTACAGTGGAGT
CCCTGATCGCTTCACAGGCAGTGGATCTGGGACAGATTTCACTCTCACCATCAGCAAT
GTGCAGTCTGAAGACTTGGCAGAGTTTTTCTGTCAGCAATATAACAACTATCCGTACA
CGTTCGGAGGGGGGACCACGCTCGAGATCAAACGAACTGTGGCTGCACCATCTGTCTT
CATCTTCCCGCCATCTGATGAGCAGTTGAAATCTGGAACTGCCTCTGTTGTGTGCCTGC
TGAATAACTTCTATCCCAGAGAGGCCAAAGTACAGTGGAAGGTGGATAACGCCCTCCA
ATCGGGTAACTCCCAGGAGAGTGTCACAGAGCAGGACAGCAAGGACAGCACCTACAG
CCTCAGCAGCACCCTGACGCTGAGCAAAGCAGACTACGAGAAACACAAAGTCTATGC
CTGCGAAGTCACCCATCAGGGCCTGAGCTCGCCCGTCACAAAGAGCTTCAACAGGGG
AGAGTGTTAG
Anti-Marco_3H10.1F3_K-V-hIgGK-C (SEQ ID NO: 135):
MESQTQVFVYMLLWLSGVDGDIVMTQSQKFMSTSLGDRVSVTCKASQNVGTNVAWYQQ
KPGHSPKALIYSASYRYSGVPDRFTGSGSGTDFTLTISNVQSEDLAEFFCQQYNNYPYTFGG
GTTLEIKRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC

The antigens of the present invention comprises one or more viral antigens or peptides from adenovirus, retrovirus, picornavirus, herpesvirus, rotaviruses, hantaviruses, coronavirus, togavirus, flavirvirus, rhabdovirus, paramyxovirus, orthomyxovirus, bunyavirus, arenavirus, reovirus, papilomavirus, parvovirus, poxvirus, hepadnavirus, or spongiform virus, HIV, CMV, hepatitis A, B, and C, influenza; measles, polio, smallpox, rubella; respiratory syncytial, herpes simplex, varicella zoster, Epstein-Barr, Japanese encephalitis, rabies, flu, or cold viruses. The antigen is selected from: Nef (66-97): VGFPVTPQVPLRPMTYKAAVDLSHFLKEKGGL (SEQ ID NO: 136); Nef (116-145): HTQGYFPDWQNYTPGPGVRYPLTFGWLYKL (SEQ ID NO: 137); Gag p17 (17-35): EKIRLRPGGKKKYKLKHIV (SEQ ID NO: 138); Gag p17-p24 (253-284): NPPIPVGEIYKRWIILGLNKIVRMYSPTSILD (SEQ ID NO: 139); and/or Pol 325-355 (RT 158-188) is: AIFQSSMTKILEPFRKQNPDIVIYQYMDDLY (SEQ ID NO: 140). In one aspect the said antigen is 19 to 32 residues and is selected from a cytotoxic T lymphocyte (CTL) epitope identified in the HIV-1 Nef, Gag and Env proteins presented in the context of MHC-class I molecules. In another aspect, the Ag is selected from HIV gp120, gp41, Gag, p17, p24, p2, p′7, p1, p6, Tat, Rev, PR, RT, IN, Vif, Vpr, Vpx, Vpu and Nef.

In another aspect the antigen is selected from tumor associated antigens selected from CEA, prostate specific antigen (PSA), HER-2/neu, BAGE, GAGE, MAGE 1-4, 6 and 12, MUC-related protein (Mucin) (MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67. In another aspect, the Ag is selected from tumor associated antigens comprising antigens from leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia.

The Ag is selected from at least one of:

(SEQ ID NO: 141)

MWVPVVFLTLSVTWIGAAPLILSRIVGGWECEKHSQPWQVLVASRGRAV
CGGVLVHPQWV;

(SEQ ID NO: 142)

LTAAHCIRNKSVILLGRHSLFHPEDTGQVFQVSHSFPHPLYDMSLLKNR
FLRPGDDSSHD;

(SEQ ID NO: 143)

LMLLRLSEPAELTDAVKVMDLPTQEPALGTTCYASGWGSIEPEEFLTPK
KLQCVDLHVIS;

(SEQ ID NO: 144)

NDVCAQVHPQKVTKFMLCAGRWTGGKSTCSGDSGGPLVCNGVLQGITS
WGSEPCALPERP;
or

(SEQ ID NO: 145)

SLYTKVVHYRKWIKDTIVANP.

In another aspect, the Ag is selected from at least one of:

(SEQ ID NO: 146)

IMDQVPFSV;

(SEQ ID NO: 147)

ITDQVPFSV;

(SEQ ID NO: 148)

YLEPGPVTV;

(SEQ ID NO: 149)

YLEPGPVTA;

(SEQ ID NO: 150)

KTWGQYWQV;

(SEQ ID NO: 151)

DTTEPATPTTPVTTPTTTKVPRNQDWLGVSRQLRTKAWNRQLYPEWTEA
QRLDCWRGGQVSLKVSNDGPTLIGANASFSIALNFPGSQKVLPDGQVIW
VNNTIINGSQVWGGQPVYPQETDDACIFPDGGPCPSGSWSQKRSFVYVW
KTWGQYWQVLGGPVSGLSIGTGRAMLGTHTMEVTVYHRRGSQSYVPLAH
SSSAFTITDQVPFSVSVSQLRALDGGNKHFLRNQ;

(SEQ ID NO: 152)

PLTFALQLHDPSGYLAEADLSYTWDFGDSSGTLISRAXVVTHTYLEPGPV
TAQVVLQAAIPLTSCGSSPVPAS;

(SEQ ID NO: 153)

GTTDGHRPTAEAPNTTAGQVPTTEVVGTTPGQAPTAEPSGTTSVQVPTT
EVISTAPVQMPTAESTGMTPEKVPVSEVMGTTLAEMSTPEATGMTPAEV
SIVVLSGTTAA;

(SEQ ID NO: 154)

QVTTTEWVETTARELPIPEPEGPDASSIMSTESITGSLGPLLDGTATLRL
VKRQVPLDCVLYRYGSFSVTLDIVQ;
and

(SEQ ID NO: 155)

GIESAEILQAVPSGEGDAFELTVSCQGGLPKEACMEISSPGCQPPAQRLC
QPVLPSPACQLVLHQILKGGSGTYCLNVSLADTNSLAVVSTQLIVPGILL
TGQEAGLGQ,
and fragments thereof.

In yet another aspect, the Ag is selected from at least one of:

(SEQ ID NO: 156)

MEMKILRALNFGLGRPLPLHFLRRASKIGEVDVEQHTLAKYLMELTML
DY;
and

(SEQ ID NO: 157)

DWLVQVQMKFRLLQETMYMTVSIIDRFMQNNCVPKK.

In another aspect, the Ag is selected from at least one of:

(SEQ ID NO: 158)

MEHQLLCCEVETIRRAYPDANLLNDRVLRAMLKAEETCAPSVSYFKCV;

(SEQ ID NO: 159)

QKEVLPSMRKIVATWMLEVCEEQKCEEEVFPLAMNYLDRFLSLEPVKK
SRLQLLGATCMFVASKMKETIPLTAEKLCIYTDNSIRPEELLQMELL;

(SEQ ID NO: 160)

LVNKLKWNLAAMTPHDFIEHFLSKMPEAEENKQIIRKHAQTFVALCATDV
KFISNPPSMV;
and

(SEQ ID NO: 161)

AGSVVAAVQGLNLRSPNNFLSYYRLTRFLSRVIKCDPDCLRACQEQIEA
LLESSLRQAQQNMDPKAAEEEEEEEEEVDLACTPTDVRDVDI,
and fragments thereof.

In another aspect, the Ag is 19 to 32 amino acids long. In another aspect, the Ag is 17 to 60 amino acids long and is selected from a cytotoxic T lymphocyte (CTL) epitope identified in PSA or cyclin 1

In another aspect, the cancer peptides are selected from tumor associated antigens selected from CEA, prostate specific antigen (PSA), HER-2/neu, BAGE, GAGE, MAGE 1-4, 6 and 12, MUC-related protein (Mucin) (MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67.

Preparation of Cells: Apheresis Procedures were Performed on Healthy Individuals after informed consent was collected. This protocol was reviewed and approved by the Baylor Research Institute Institutional Review Board. PBMCs were purified from apheresis blood samples and used after cryopreservation. Monocyte-derived IFNa-DCs were prepared from frozen human monocytes (elutriation fraction 5, Lemarie et al, J. Immunological Methods, 2007) cultured with GM-CSF (100 ng/ml) and IFNa (500 U/ml)) (Salluto et al, J. Exp. Med) for 3 days in Cellgenix.

Extracellular cytokine secretion assay. PBMCs or monocyte-derived IFNa-DCs (2×10⁶ cells/ml, 200 μl/well) were cultured in cRPMI containing 10% human AB serum, 2 mM L-glutamine, 50 U penicillin, 50 μg/ml streptomycin, 1× essential amino acids, 25 mM hepes, 55 μLLM 2-mercapto-ethanol with DC-targeting vaccines and TLR ligands of interest or left unstimulated (negative control) for 24 h, at 37° C. and 5% CO2. Then culture supernatants were harvested and then the secreted cytokines were measured in the culture supernatants using BioPlex200 Luminex (BioRad).

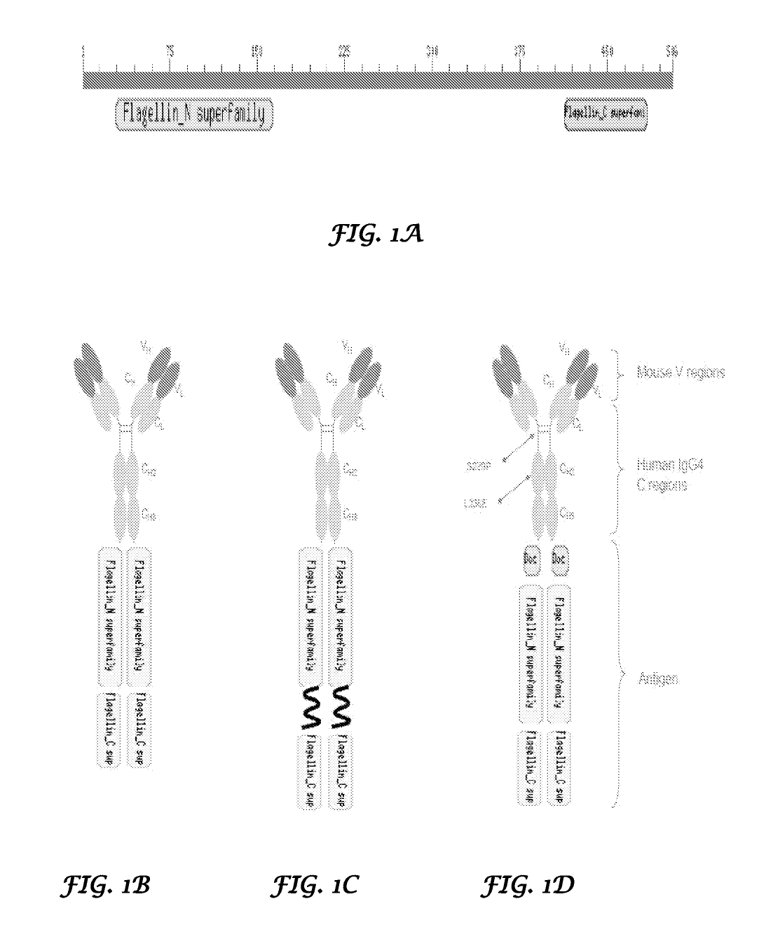

FIGS. 1A to 1D shows flagellin and the several antibody-flagellin contructs of the present invention.

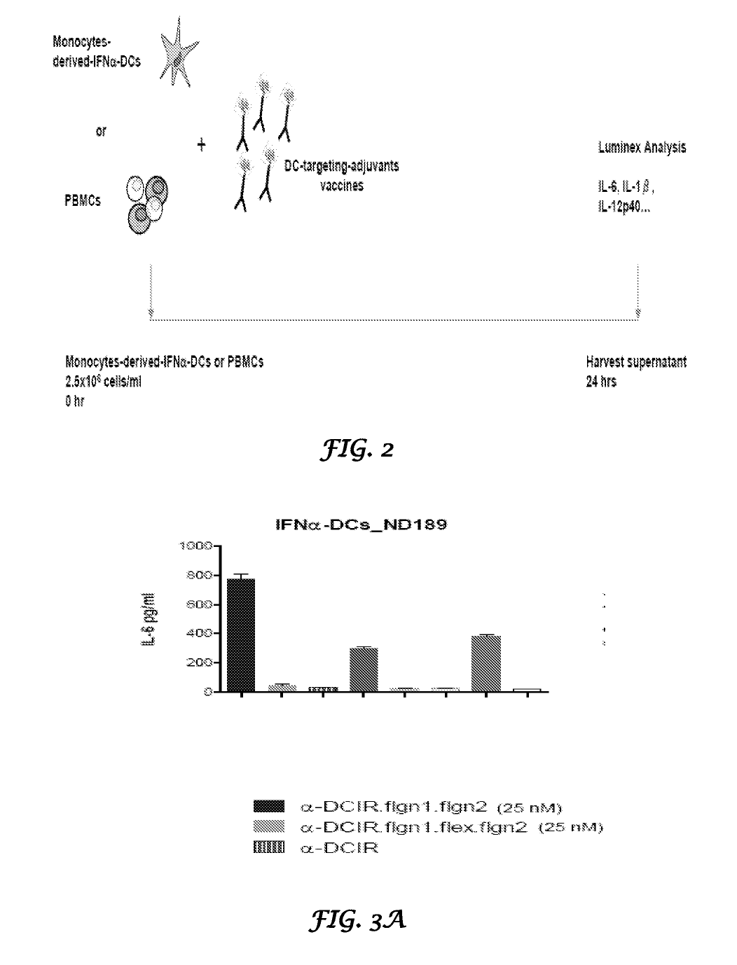

FIG. 2 shows the design of the studies used to test the activity of the antibody-flagellin constructs of the present invention.

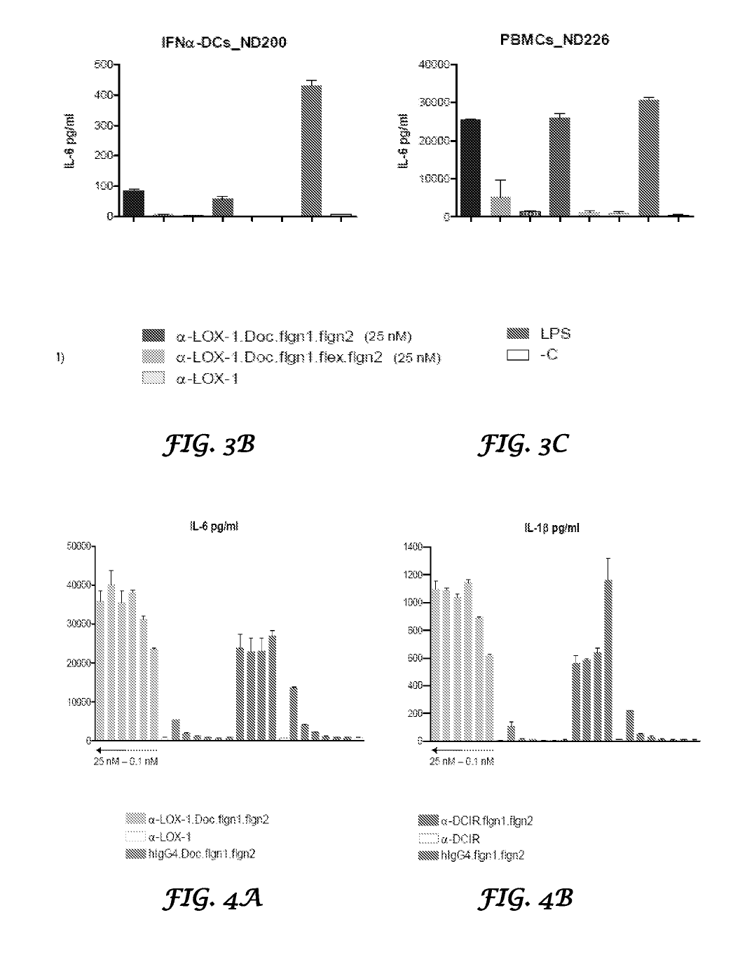

FIGS. 3A to 3C show the secretion of IL-6 when various types of cells were activated with the construction of the present invention. The addition of the flexible linker (flex) between the 2 flagellin domains (flgn1 and flgn2) abolishes the activity. The various cells were IFNalpha activated DCs and Peripheral Blood Mononuclear Cells (PBMCs).

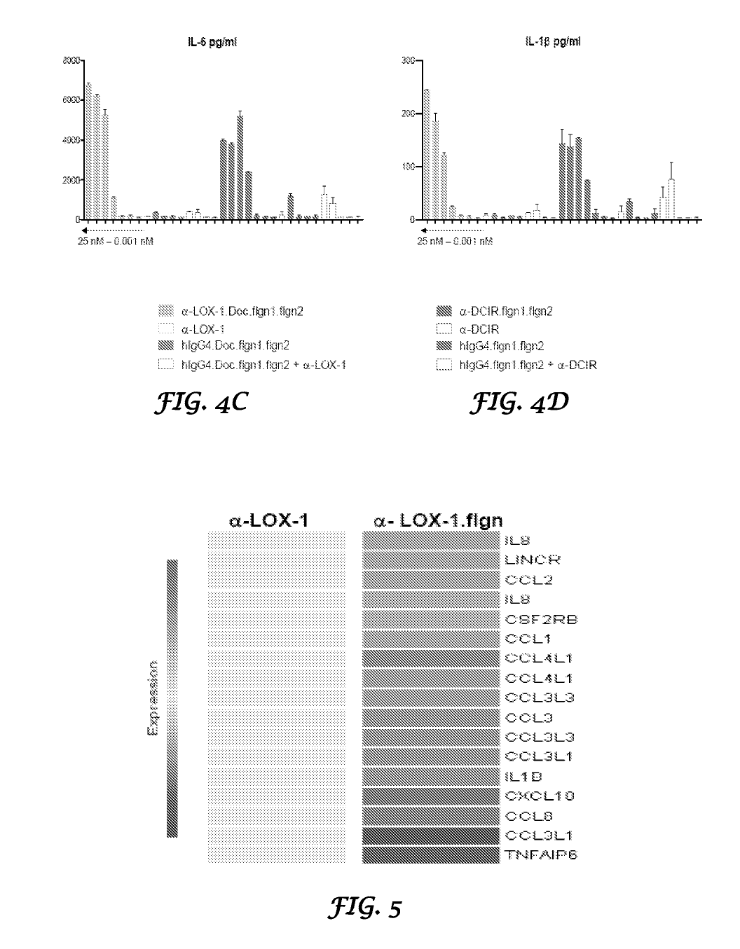

FIGS. 4A to 4B show the dose titration of the various constructs as measured by the secretion of IL-6 and IL-1 beta. The flagellin activity is dependent on the targeting antibody and is effective even at the lowest 0.1 nM dose, while the isotype control gives only a response at 25 nM dose. FIGS. 4C and 4D show the dose titration of the various constructs as measured by the secretion of IL-6 and IL-1 beta. The flagellin activity is dependent on the targeting antibody and is effective even at the lowest 0.1 nM dose, while the isotype control gives only a response at 25 nM dose. Moreover, the addition of free antibody to the isotype control does not restore the flagellin activity.

FIG. 5 shows heat maps of gene expression for the listed genes using an anti-LOX-1 antibody with or without flagellin. Of the heat maps obtained from IFNα-DCs cultured with α-LOX-1.flgn, it was found that 17 transcripts are overexpressed in response to the αLOX-1.flgn stimuli.

It is contemplated that any embodiment discussed in this specification can be implemented with respect to any method, kit, reagent, or composition of the invention, and vice versa. Furthermore, compositions of the invention can be used to achieve methods of the invention.

It may be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it may be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Patent Application Publication No. 20090004194: TLR Agonist (Flagellin)/CD40 Agonist/Antigen Protein and DNA Conjugates and use Thereof for Inducing Synergistic Enhancement in Immunity.

U.S. Patent Application Publication No. 20080220011: Use of Flagellin in Tumor Immunotherapy.

U.S. Patent Application Publication No. 20080248068: Use of Flagellin as an Adjuvant for Vaccine.

U.S. Pat. No. 7,404,963: Flagellin-Based Adjuvants and Vaccines.

What is claimed is:

1. A composition comprising:
an anti-dendritic cell (DC)-specific antibody or binding fragment thereof conjugated to a TLR agonist; and at least one antigen, wherein the antigen and the agonist are effective to produce an immune response in a human or animal subject in need of immunostimulation.

2. The composition of claim 1, wherein the DC-specific antibody or fragment is selected from an anti-DCIR, MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, or ASPGR.

3. The composition of claim 1, wherein the composition further comprises antigenic peptides selected from human immunodeficiency virus (HIV) antigens and gene products selected from the group consisting of gag, pol, and env genes, the Nef protein, reverse transcriptase, string of HIV peptides (Hipo5), PSA (KLQCVDLHV)-tetramer, a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide (GILGFVFTL) tetramer, and Avian Flu (HA5-1), dockerin domain from C. thermocellum, measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens or combinations and modifications thereof.

4. The composition of claim 1, wherein the composition further comprises antigenic peptides selected from one or more bacterial antigens, wherein the bacterial antigens comprise antigens derived from Bacillus, Escherichia, Listeria, Neisseria, Nocardia, Salmonella, Staphylococcus, Streptococcus, or combinations and modifications thereof.

5. The composition of claim 1, wherein the composition further comprises antigenic peptides selected from cancer peptides selected from tumor associated antigens comprising antigens from leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia.

6. The composition of claim 1, wherein the composition further comprises antigenic peptides selected from tumor associated antigens selected from CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67.

7. The composition of claim 1, wherein the DC-specific antibody is humanized.

8. The composition of claim 1, wherein the TLR agonist comprises at least one of a flagellin from Salmonella enterica, a flagellin from Vibrio cholerae, any other TLR5 agonist, a TLR7 agonist, a TLR9 agonist, or any combinations or modifications thereof.

9. The composition of claim 1, wherein the antigen is conjugated to the antibody and TLR agonist.

10. The composition of claim 1, wherein the antigen and the antibody are a single fusion protein.

11. The composition of claim 1, wherein the antibody comprises at least one of a light chain, a heavy chain, or a heavy and a light chain.

12. A vaccine composition comprising:
an antigen; and
an adjuvant, wherein the adjuvant comprises an anti-dendritic cell (DC)-specific antibody or fragment thereof conjugated to at least a portion of a TLR agonist in an amount effective to produce an immune response in a human or animal subject in need of immunostimulation.

13. The composition of claim 12, wherein the DC-specific antibody or fragment is selected from an anti-DCIR, MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, or ASPGR.

14. The composition of claim 12, wherein the composition further comprises antigenic peptides selected from human immunodeficiency virus (HIV) antigens and gene products selected from the group consisting of gag, pol, and env genes, the Nef protein, reverse transcriptase, string of HIV peptides (Hipo5), PSA (KLQCVDLHV)-tetramer, a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide (GILGFVFTL) tetramer, and Avian Flu (HA5-1), dockerin domain from C. thermocellum, measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens or combinations and modifications thereof.

15. The composition of claim 12, wherein the composition further comprises antigenic peptides selected from one or more bacterial antigens, wherein the bacterial antigens comprise antigens derived from Bacillus, Escherichia, Listeria, Neisseria, Nocardia, Salmonella, Staphylococcus, Streptococcus, or combinations and modifications thereof.

16. The composition of claim 12, wherein the composition further comprises antigenic peptides selected from cancer peptides selected from tumor associated antigens comprising antigens from leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia.

17. The composition of claim 12, wherein the composition further comprises antigenic peptides selected from tumor associated antigens selected from CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67.

18. The composition of claim 12, wherein the DC-specific antibody is humanized.

19. The composition of claim 12, wherein the TLR agonist comprises at least one of a flagellin from Salmonella enterica, a flagellin from Vibrio cholerae, any other TLR5 agonist, a TLR7 agonist, a TLR9 agonist, or any combinations or modifications thereof.

20. The composition of claim 12, wherein the antigen is conjugated to the antibody and TLR agonist.

21. The composition of claim 12, wherein the antigen and the antibody are a single fusion protein.

22. The composition of claim 12, wherein the antibody comprises at least one of a light chain, a heavy chain, or a heavy and a light chain.

23. A method for increasing effectiveness of antigen presentation by an antigen presenting cell comprising:
contacting the antigen presenting cell with a composition comprising:
an antigen; and
an adjuvant, wherein the adjuvant comprises an anti-dendritic cell (DC)-specific antibody or binding fragment thereof conjugated to a TLR agonist, wherein the antigen and adjuvant are provided in an amount effective to produce an immune response in a human or animal subject in need of immunostimulation.

24. The method of claim 23, wherein the DC-specific antibody or fragment is selected from an anti-DCIR, MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, or ASPGR.

25. The method of claim 23, wherein the anti-DC-specific antibody is selected from pairs of SEQ ID NOS.: 7 and 9, 11 and 13, 15 and 17, 19 and 21, 23 and 25, 27 and 29, 31 and 33, 35 and 37, 39 and 41, 43 and 45, 47 and 49, 51 and 53, 55 and 57, 59 and 61, 63 and 65, 67 and 69, 71 and 73, 75 and 77, 79 and 81, 83 and 85, 87 and 89, 91 and 93, 95 and 97, 99 and 101, 1-3 and 105, 107 and 109, 111 and 113, 115 and 117, 119 and 121, 123 and 125, 127 and 129, 131 and 132, 133 and 134.

26. The method of claim 23, wherein the composition further comprises antigenic peptides selected from human immunodeficiency virus (HIV) antigens and gene products selected from the group consisting of gag, pol, and env genes, the Nef protein, reverse transcriptase, string of HIV peptides (Hipo5), PSA (KLQCVDLHV)-tetramer, a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide (GILGFVFTL) tetramer, and Avian Flu (HA5-1), dockerin domain from C. thermocellum, measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens or combinations and modifications thereof.

27. The method of claim 23, wherein the composition further comprises antigenic peptides selected from cancer peptides selected from tumor associated antigens comprising antigens from leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia.

28. The method of claim 23, wherein the composition further comprises antigenic peptides selected from tumor associated antigens selected from CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67.

29. The method of claim 23, wherein the DC-specific antibody is humanized.

30. The method of claim 23, wherein the composition is administered to the human or animal subject by an oral route, a nasal route, topically or as an injection.

31. The method of claim 23, wherein the injection is selected from the group consisting of intradermal, intramucosal, subcutaneous, intravenous, intraperitoneal, intramuscular, and intravenous.

32. The method of claim 23, wherein the TLR agonist comprises at least one of a flagellin from Salmonella enterica, a flagellin from Vibrio cholerae, any other TLR5 agonist, a TLR7 agonist, a TLR9 agonist, or any combinations or modifications thereof.

33. The method of claim 23, wherein the antigen is conjugated to the antibody and TLR agonist.

34. The method of claim 23, wherein the antigen and the antibody are a single fusion protein.

35. The method of claim 23, wherein the antibody comprises at least one of a light chain, a heavy chain, or a heavy and a light chain.

36. A method for a treatment, a prophylaxis or a combination thereof against one or more cancers in a human subject comprising the steps of:
identifying the human subject in need of the treatment, the prophylaxis or a combination thereof against the one or more cancers; and
administering a vaccine composition comprising:
an antigen; and
an adjuvant, wherein the adjuvant comprises an anti-dendritic cell (DC)-specific antibody or fragment thereof conjugated to a TLR agonist; and a pharmaceutically acceptable carrier, wherein the antigen and adjuvant are provided in an amount effective to produce an immune response for the treatment, the prophylaxis or a combination thereof against the one or more cancers.

37. The method of claim 36, wherein the composition is administered to the human or animal subject by an oral route, a nasal route, topically or as an injection.

38. The method of claim 37, wherein the injection is selected from the group consisting of intradermal, intramucosal, subcutaneous, intravenous, intraperitoneal, intramuscular, and intravenous.

39. A method of providing immunostimulation by activation of one or more dendritic cells (DCs) to a human subject for a prophylaxis, a therapy or a combination thereof against one or more viral, bacterial, fungal, parasitic, protozoal, and parasitic diseases, and allergic disorders comprising the steps of:
identifying the human subject in need of immunostimulation for the prophylaxis, the therapy or a combination thereof against the one or more viral, bacterial, fungal, parasitic, protozoal, and parasitic diseases, and allergic disorders;
isolating one or more DCs from the human subject;
activating the isolated DCs with an amount of a composition effective for forming activated DCs comprising:
an antigen; and
an adjuvant, wherein the adjuvant comprises an anti-dendritic cell (DC)-specific antibody or fragment thereof conjugated to a TLR agonist; and a pharmaceutically acceptable carrier, in an amount effective to produce an immune response in a human or animal subject in need of immunostimulation; and
reintroducing the activated DCs into the human subject.

40. An adjuvant composition comprising:
an anti-dendritic cell (DC)-specific antibody or fragment thereof conjugated to at least a portion of a TLR agonist wherein the anti-DC-specific antibody is selected from pairs of SEQ ID NOS.: 7 and 9, 11 and 13, 15 and 17, 19 and 21, 23 and 25, 27 and 29, 31 and 33, 35 and 37, 39 and 41, 43 and 45, 47 and 49, 51 and 53, 55 and 57, 59 and 61, 63 and 65, 67 and 69, 71 and 73, 75 and 77, 79 and 81, 83 and 85, 87 and 89, 91 and 93, 95 and 97, 99 and 101, 1-3 and 105, 107 and 109, 111 and 113, 115 and 117, 119 and 121, 123 and 125, 127 and 129, 131 and 132, 133 and 134.

41. The composition of claim 40, wherein the composition further comprises antigenic peptides selected from human immunodeficiency virus (HIV) antigens and gene products selected from the group consisting of gag, pol, and env genes, the Nef protein, reverse transcriptase, string of HIV peptides (Hipo5), PSA (KLQCVDLHV)-tetramer, a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide (GILGFVFTL) tetramer, and Avian Flu (HA5-1), dockerin domain from C. thermocellum, measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens or combinations and modifications thereof.

42. The composition of claim 40, wherein the composition further comprises antigenic peptides selected from cancer peptides selected from tumor associated antigens comprising antigens from leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia.

43. The composition of claim 40, wherein the composition further comprises antigenic peptides selected from tumor associated antigens selected from CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67.

44. The composition of claim 40, wherein the DC-specific antibody is humanized.

45. The composition of claim 40, wherein the TLR agonist comprises at least one of a flagellin from Salmonella enterica, a flagellin from Vibrio cholerae, any other TLR5 agonist, a TLR7 agonist, a TLR9 agonist, or any combinations or modifications thereof.

46. The composition of claim 40, wherein the antigen and the antibody are a single fusion protein.

47. The composition of claim 40, wherein the antibody comprises at least one of a light chain, a heavy chain, or a heavy and a light chain.
